(19) United States
(12) Patent Application Publication
(10) Pub. No.: US 2023/0183314 A1
(43) Pub. Date: Jun. 15, 2023

(54) PROSTATE SPECIFIC MEMBRANE ANTIGEN BINDING FIBRONECTIN TYPE III DOMAINS AND CELLS COMPRISING THE SAME

(71) Applicant: ARO BIOTHERAPEUTICS COMPANY, Philadelphia, PA (US)

(72) Inventor: Karyn O'NEIL, Philadelphia, PA (US)

(21) Appl. No.: 17/923,475

(22) PCT Filed: May 5, 2021
(86) PCT No.: PCT/US2021/030863

§ 371 Date: Nov. 4, 2022

Related U.S. Application Data
(60) Provisional application No. 63/020,363, filed on May 5, 2020.

(51) Int. Cl. C07K 14/725; C12N 5/0786; C07K 14/47; C07K 14/735; C07K 14/705; A61K 35/15; A61P 35/00; A61P 13/08

(52) U.S. Cl. CPC C07K 14/7051; C12N 5/0645; C07K 14/47; C07K 14/70535; C07K 14/70596; A61K 35/15; A61P 35/00; A61P 13/08; A61K 38/00

(57) ABSTRACT

Cells such as macrophages comprising chimeric antigen receptors comprising PSMA binding FN3 domains, their conjugates, isolated nucleotides encoding the molecules, vectors, host cells, and methods of making thereof are useful in the generation of therapeutic molecules and treatment and diagnosis of diseases and disorders.

### CROSS-REFERENCE TO RELATED APPLICATIONS

This application claims priority to U.S. Provisional Application No. 63/020,363, filed May 5, 2020, which is hereby incorporated by reference in its entirety

### FIELD

The present embodiments related to macrophages or cells comprising prostate specific membrane antigen binding molecules and methods of making and using the molecules.

### BACKGROUND

Prostate specific membrane antigen (PSMA), also known as glutamate carboxypeptidase II or N-acetylated alpha-linked acidic dipeptidase 1, is a dimeric type 2 transmembrane glycoprotein. PSMA cleaves several substrates, including folate and N-acetyl-L-aspartyl-L-glutamate, and is expressed in a number of tissues with highest expression in prostate, and to a lesser extent in the small intestine, central and peripheral nervous system, kidney and lung. PSMA is constitutively internalized through clathrin coated pits.

PSMA is a prostate-cancer related cell membrane antigen frequently overexpressed in prostatic intraepithelial neoplasia (PIN), a condition in which some prostate cells have begun to look and behave abnormally, primary and metastatic prostate cancers and the neovasculature of other solid tumors, (e.g. breast, lung, bladder, kidney). PSMA expression correlates with disease progression and Gleason score. PSMA expression is increased in metastatic disease, hormone refractory cases, and higher-grade lesions, and it is further upregulated in androgen-insensitive tumors.

Prostate cancer is the leading cause of cancer among males, and the 2^{nd} leading cause of cancer-induced death. Globally, there are approximately 1,100,000 new cases and 300,000 mortalities every year, translating to about 4% of all cancer deaths. It is estimated that 1 in every 6 men will be diagnosed with the disease. In the U.S., more than 90% of prostate cancers are found in local or regional stages. At these early stages, the 5-year survival rate is close to 100%. When the cancer has metastasized, however, the 5-year survival rate is reduced to about 28%. Localized prostate cancer can often be controlled by hormone deprivation.

Current treatments for prostate cancer include surgery, radiation and hormone therapies. However, tumor cells often become androgen insensitive, and limited treatment options remain. Typically, the cancer vaccine sipuleucel-T, a radiopharmaceutical agent (such as radium-223 chloride), secondary hormone therapies (such as abiraterone or enzalutamide), and/or chemotherapies (docetaxel and cabazitaxel) are added to the hormonal therapy in sequence.

Monoclonal antibodies targeting PSMA have been evaluated in the clinic as both diagnostic imaging agents and antibody-drug conjugates. The most extensively evaluated antibody-drug conjugates (ADCs) targeting PSMA utilize the same humanized/de-immunized anti-PSMA mAb, J591. At least three different ADCs utilizing J591 have been evaluated in clinical trials, utilizing various linkers and warheads. Millenium Pharmaceuticals completed Phase 1 clinical trials evaluating MLN2704, an anti-PSMA mAb conjugated to a disulfide linked maytansine; Progenics utilized Seattle Genetics technology to link J591 to MMAE using a valine-citrulline linker, and ADCT is initiating clinical trials for a PBD-conjugated to J591. To date, limited clinical efficacy has been coupled with serious toxicities and short serum half-lives, likely due to significant liver uptake. Nonetheless, in two separate clinical studies, there was evidence of decreased PSA/CTC levels following repeat treatment with anti-PSMA ADCs, particularly at the higher doses. In the case of Progenics, two dose-limiting toxicities resulted in death following sepsis due to neutropenia.

While each of these treatments can delay growth of the cancer for several months and palliate symptoms produced by the disease, the disease ultimately becomes resistant to them.

Therefore, there is a need for additional and improved therapeutics to treat prostate cancer and other cancers overexpressing PSMA.

A cell comprising a polypeptide that comprises at least one heterologous fibronectin type III (FN3), wherein the at least one heterologous FN3 domain is on the surface of the cell and binds to human prostate specific membrane antigen (PSMA).

### SUMMARY

In some embodiments, cells comprising a fibronectin type III (FN3) domain on its surface wherein the cell targets and binds a cell expressing a human prostate specific membrane antigen (PSMA-expressing cell) are provided.

In some embodiments, cells comprising a polypeptide that comprises at least one heterologous fibronectin type III (FN3) domain, wherein the at least one heterologous FN3 domain is on the surface of the cell and binds to human prostate specific membrane antigen (PSMA) are provided.

In some embodiments, the polypeptide comprises a first FN3 domain and a second FN3 domain, wherein the first and second FN3 domains each bind to human prostate specific membrane antigen (PSMA). In some embodiments, the first and second FN3 domains bind to different epitopes of PSMA.

In some embodiments, macrophages comprising a chimeric antigen receptor comprising at least one heterologous fibronectin type III (FN3) domain, wherein the at least one heterologous FN3 domain is on the surface of the cell and binds to human prostate specific membrane antigen (PSMA), operably connected to a transmembrane domain and an intracellular domain of a stimulatory and/or co-stimulatory molecule are provided.

In some embodiments, chimeric antigen receptor or a polypeptide comprising a polypeptide having the formula of A1-L-A2 operably connected to a transmembrane domain and an intracellular domain of a stimulatory and/or co-stimulatory molecule are provided, wherein A1, L, and A2 are as provided for herein.

In some embodiments, pharmaceutical compositions comprising the cells, macrophages, or the polypeptides provided for herein are provided.

Other embodiments directed to methods of treating prostate cancer in a subject comprising administering the cell or a pharmaceutical composition comprising the same as described herein are provided. In some embodiments, the cell is a macrophage.

Other embodiments directed to methods of treating prostate cancer in a subject comprising administering a pharmaceutical composition comprising a polypeptide or chimeric antigen receptor as described herein are provided.

### BRIEF DESCRIPTION OF THE DRAWINGS

FIG. 1 shows biodistribution of untargeted ⁸⁹Zr-labeled Centyrin following intravenous injection in male NSG mice.

FIG. 2A shows the overall crystal structure of the P233FR9_H10 PSMA binding FN3 domain (H10) in complex with cynomolgous PSMA dimer, showing that H10 binds to the region near the PSMA active site. The zinc atoms (Zn) indicate the location of the PSMA active site. The N- and C-terminus of PSMA and H10 molecules are indicated for one of the complexes. The approximate location of the cell membrane is indicated.

FIG. 2B shows the crystal structure of the H10 FN3 domain in complex with cynomolgous PSMA. The A, B, C, D, E, F and G beta strands in the H10 FN3 domain are shown. The negatively charged residues in the CD loop of H10 (residues W38, D39, D40, D41 and E43) that inserts into the positively charged entrance of the PSMA active site are shown. H10 residue numbering according to SEQ ID NO: 41.

FIG. 2C shows the crystal structure of the H10 FN3 domain in complex with cynomolgous PSMA. The H10 contact residues W38, D39, D40, D41 and E43 are shown in the Figure. Some of the cyno PSMA residues that contact H10 (R511, K514 and K545), coordinate the zinc atoms (H377, D387, E424, E425, D453, and H553) or compose the active site cavity (R536 and R534) are shown. H10 beta strands C, D, F and G are marked in the Figure. H10 and cynomolgous PSMA residue numbering is according to SEQ ID NO: 41 and 141, respectively.

FIG. 3A shows a close view of the crystal structure combining site between the H10 FN3 domain and cynomolgous PSMA. The H10 FN3 domain contact residues A32, W36, W38-D41, E43, A44, V46, G64, P68, Y70, A72, W79, F81, P82, A85, and 186 are shown. The cyno PSMA contact residues Y460, K499-P502, P504, R511, K514, N540, W541, K545, F546, F488, K610, N613, and I614 are shown. H10 and cynomolgous PSMA residue numbering is according to SEQ ID NO: 41 and 141, respectively.

FIG. 3B shows an interaction map between the H10 FN3 domain and cynomolgous PSMA contact residues. A distance cut-off of 4 Å was used to define the contact residues. Centyrin and cyno PSMA residues are shown in gray and white boxes, respectively, van der Waals interactions are shown as dashed lines, and H-bonds are solid lines with arrows indicating backbone H bonds and pointing to the backbone atoms. Residue numbering is according to SEQ ID NO: 41 (H10) and SEQ ID NO: 141 (cyno PSMA).

FIG. 4A shows the amino acid sequence alignment between human (h) and cynomolgous (c) PSMA extracellular domains. The H10 contact residues are underlined and in bold. The residues that differ between human and cynomolgus PSMA are shaded. All cyno PSMA residues interacting with H10 are conserved in human PSMA except for N613. Human PSMA ECD; SEQ ID NO: 143. Cyno PSMA ECD: SEQ ID NO: 32

FIG. 4B shows the H10 FN3 domain residues in contact with cynomolgous PSMA. The contact residues are underlined and in bold. H10 amino acid sequence is shown in SEQ ID NO: 41.

FIG. 5 shows the location of H10 centyrin residue N6, R11, T22, D25, A26, S52, E53, K62, and the N- and C-terminus, which are possible sites for chemical conjugation, in the crystal structure of H10 bound to cynomolgous PSMA. The centyrin/PSMA contacting regions are shown in black. H10 beta strands C, D, F and G are marked in the Figure. Residue numbering according to SEQ ID NO: 41 (H10).

FIG. 6 shows the comparison of mean fluorescence intensity (MFI) of different tumor cell lines stained with anti PSMA centyrin-PE (black) and anti PSMA antibody-PE (white).

FIG. 7A shows a series of the CellTracks Analyzer II browser images of LNCaP cells stained with DAPI, anti-cytokeratin-FITC, anti CD45-APC and anti PSMA centyrin-PE. The thumbnail images show, from right to left, PSMA-PE staining, CD45-APC signal, DAPI stained nuclei, Cytokeratin-FITC reactivity, and finally an overlay of the Cytokeratin-FITC & DAPI staining. A cell must have a nucleus, express cytokeratin and be negative for CD45 to be counted as a CTC. The CTC must have a positive signal for PSMA to be scored as PSMA positive CTC.

FIG. 7B shows a series of the CellTracks Analyzer II browser images of 22Rv1 cells stained with DAPI, anti-cytokeratin-FITC, anti CD45-APC and anti PSMA centyrin-PE. The thumbnail images show, from right to left, PSMA-PE staining, CD45-APC signal, DAPI stained nuclei, Cytokeratin-FITC reactivity, and finally an overlay of the Cytokeratin-FITC & DAPI staining. A cell must have a nucleus, express cytokeratin and be negative for CD45 to be counted as a CTC. The CTC must have a positive signal for PSMA to be scored as PSMA positive CTC.

FIG. 7C shows shows a series of the CellTracks Analyzer II browser images of PC3 cells stained with DAPI, anti-cytokeratin-FITC, anti CD45-APC and anti PSMA centyrin-PE. The thumbnail images show, from right to left, PSMA-PE staining, CD45-APC signal, DAPI stained nuclei, Cytokeratin-FITC reactivity, and finally an overlay of the Cytokeratin-FITC & DAPI staining. A cell must have a nucleus, express cytokeratin and be negative for CD45 to be counted as a CTC. The CTC must have a positive signal for PSMA to be scored as PSMA positive CTC.

FIG. 7D shows shows a series of the CellTracks Analyzer II browser images of SKBR3 cells stained with DAPI, anti-cytokeratin-FITC, anti CD45-APC and anti PSMA centyrin-PE. The thumbnail images show, from right to left, PSMA-PE staining, CD45-APC signal, DAPI stained nuclei, Cytokeratin-FITC reactivity, and finally an overlay of the Cytokeratin-FITC & DAPI staining. A cell must have a nucleus, express cytokeratin and be negative for CD45 to be counted as a CTC. The CTC must have a positive signal for PSMA to be scored as PSMA positive CTC.

FIG. 8A shows mRNA levels for LnCAP cells treated with increasing doses of H10-Centyrin-β-catenin siRNA conjugate in the absence of transfection reagent.

FIG. 8B shows mRNA levels for 22Rv1 cells treated with increasing doses of H10-Centyrin-β-catenin siRNA conjugate in the absence of transfection reagent.

FIG. 8C shows dose dependent binding of H10 to LnCAP cells measured by flow cytometry.

FIG. 8D shows dose dependent cytotoxicity of H10-MMAF conjugates in LnCAP cells.

FIG. 8E shows dose dependent cytotoxicity of H10-MMAF conjugates in 22Rv1 cells.

FIG. 9A shows dose dependent knockdown of β catenin mRNA by H10-siRNA (black filled squares) or D02 siRNA (red filled triangles) conjugates in LnCAP cells.

FIG. 9B shows dose dependent knockdown of β catenin mRNA by H10-siRNA (black filled squares) or D02 siRNA (red filled triangles) conjugates in 22Rv1 cells

FIG. 9C shows dose dependent binding of H10 (black filled squares) and D02 (red filled triangles) to LnCAP cells assessed by flow cytometry.

FIG. 9D compares dose dependent cytotoxicity of H10-MMAF (black filled squares) and D02-MMAF (red filled triangles) Centyrin-toxin conjugates in LnCAP cells.

FIG. 9E compares dose dependent cytotoxicity of H10-MMAF (black filled squares) and D02-MMAF (red filled triangles) Centyrin-toxin conjugates in 22Rv1 cells.

FIG. 10A compares dose dependent binding of H10 (black filled squares) or H10v18 (red filled triangles) to LnCAP cells assessed by flow cytometry.

FIG. 10A illustrates a various embodiment provided for herein.

FIG. 10B compares dose dependent cytotoxicity of H10 MMAF (black filled squares) or H10v18 MMAF (red filled triangles) Centyrin-toxin conjugates on LnCAP cells.

FIG. 10C compares dose dependent cytotoxicity of H10 MMAF (black filled squares) or H10v18 MMAF (red filled triangles) Centyrin-toxin conjugates on 22Rv1 cells.

FIG. 11 shows tumor growth profiles for LnCAP tumor xenografts in mice. Mice were treated with either vehicle (black filled diamonds), 10 mg/kg Tencon-MMAF conjugate (red filled inverted triangles) or 10 mg/kg H10-MMAF conjugate (blue filled squares) on days 1, 8 and 15.

### DETAILED DESCRIPTION

The term “fibronectin type III (FN3) domain” (FN3 domain) as used herein refers to a domain occurring frequently in proteins including fibronectins, tenascin, intracellular cytoskeletal proteins, cytokine receptors and prokaryotic enzymes (Bork and Doolittle, Proc Nat Acad Sci USA 89:8990-8994, 1992; Meinke et al., J Bacteriol 175:1910-1918, 1993; Watanabe et al., J Biol Chem 265:15659-15665, 1990). Exemplary FN3 domains are the 15 different FN3 domains present in human tenascin C, the 15 different FN3 domains present in human fibronectin (FN), and non-natural synthetic FN3 domains as described for example in U.S. Pat. No. 8,278,419. Individual FN3 domains are referred to by domain number and protein name, e.g., the 3^{rd} FN3 domain of tenascin (TN3), or the 10^{th} FN3 domain of fibronectin (FN10).

“Centyrin” as used herein refers to a FN3 domain that is based on the consensus sequence of the 15 different FN3 domains present in human tenascin C.

The term “capture agent” refers to substances that bind to a particular type of cells and enable the isolation of that cell from other cells. Examples of capture agents include but are not limited to magnetic beads, ferrofluids, encapsulating reagents and the like.

The term “biological sample” refers to blood, tissue, marrow, sputum and the like.

The term “diagnostic reagent” refers to any substance that may be used to analyze a biological sample, whether or not such substance is distributed as a single substance or in a combination with other substances in a diagnostic kit.

The term “substituting” or “substituted” or ‘mutating” or “mutated” as used herein refers to altering, deleting of inserting one or more amino acids or nucleotides in a polypeptide or polynucleotide sequence to generate a variant of that sequence.

The term “randomizing” or “randomized” or “diversified” or “diversifying” as used herein refers to making at least one substitution, insertion or deletion in a polynucleotide or polypeptide sequence.

“Variant” as used herein refers to a polypeptide or a polynucleotide that differs from a reference polypeptide or a reference polynucleotide by one or more modifications for example, substitutions, insertions or deletions.

The term “specifically binds” or “specific binding” as used herein refers to the ability of the FN3 domain of the invention to bind to a predetermined antigen with a dissociation constant (K_{D}) of about 1×10⁻⁶ M or less, for example about 1×10⁻⁷ M or less, about 1×10⁻⁸ M or less, about 1×10⁻⁹ M or less, about 1×10⁻¹° M or less, about 1×10⁻¹¹ M or less, about 1×10⁻¹² M or less, or about 1×10⁻¹³ M or less. Typically the FN3 domain of the invention binds to a predetermined antigen (i.e. human PSMA) with a K_{D} that is at least ten fold less than its K_{D} for a nonspecific antigen (for example BSA or casein) as measured by surface plasmon resonance using for example a Proteon Instrument (BioRad). The isolated FN3 domain of the invention that specifically binds to human PSMA may, however, have cross-reactivity to other related antigens, for example to the same predetermined antigen from other species (homologs), such as Macaca Fascicularis (cynomolgous monkey, cyno) or Pan troglodytes (chimpanzee).

The term “epitope” as used herein means a portion of an antigen to which an FN3 domain of the invention specifically binds. Epitopes usually consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

The term “library” refers to a collection of variants. The library may be composed of polypeptide or polynucleotide variants.

The term “stability” as used herein refers to the ability of a molecule to maintain a folded state under physiological conditions such that it retains at least one of its normal functional activities, for example, binding to a predetermined antigen such as human PSMA.

Human PSMA as used herein refers to the well-known type II glycoprotein of about 100 kD with a short intracellular domain (residues 1-18), a transmembrane domain (residues 19-43) and an extracellular domain (residues 44-750). The amino acid sequence of the mature human PSMA is shown in SEQ ID NO: 144.

“Overexpress”, “overexpressed” and “overexpressing” as used herein interchangeably refer to a cancer or malignant cell that has measurably higher levels of PSMA on the surface compared to a normal cell of the same tissue type. Such overexpression may be caused by gene amplification or by increased transcription or translation. PSMA overexpression can be measured using well know assays using for example ELISA, immunofluorescence, flow cytometry or radioimmunoassay on live or lysed cells. Alternatively, or additionally, levels of PSMA nucleic acid molecules may be measured in the cell for example using fluorescent in situ hybridization, Southern blotting, or PCR techniques. PSMA is overexpressed when the level of PSMA on the surface of the cell is at least 1.5-fold higher when compared to the normal cell.

“Tencon” as used herein refers to the synthetic fibronectin type III (FN3) domain having the sequence shown in SEQ ID NO: 1 and described in U.S. Pat. Publ. No. US2010/0216708.

A “cancer cell” or a “tumor cell” as used herein refers to a cancerous, pre-cancerous or transformed cell, either in vivo, ex vivo, and in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is exemplified by, e.g., morphological changes, immortalization of cells, aberrant growth control, foci formation, proliferation, malignancy, tumor specific markers levels, invasiveness, tumor growth or suppression in suitable animal hosts such as nude mice, and the like, in vitro, in vivo, and ex vivo (Freshney, Culture of Animal Cells: A Manual of Basic Technique (3rd ed. 1994)).

“Inhibits growth” (e.g. referring to cells, such as tumor cells) refers to a measurable decrease in the cell growth in vitro or in vivo when contacted with a therapeutic or a combination of therapeutics or drugs when compared to the growth of the same cells grown in appropriate control conditions well known to the skilled in the art. Inhibition of growth of a cell in vitro or in vivo may be at least about 10%, 20%, 30%, 40%, 50%, 60%, 70%, 80%, 90%, 99%, or 100%. Inhibition of cell growth may occur by a variety of mechanisms, for example by apoptosis, necrosis, or by inhibition of cell proliferation, or lysis of cells.

The term “vector” means a polynucleotide capable of being duplicated within a biological system or that can be moved between such systems. Vector polynucleotides typically contain elements, such as origins of replication, polyadenylation signal or selection markers that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules or a hybrid of these.

The term “expression vector” means a vector that can be utilized in a biological system or in a reconstituted biological system to direct the translation of a polypeptide encoded by a polynucleotide sequence present in the expression vector.

The term “polynucleotide” means a molecule comprising a chain of nucleotides covalently linked by a sugar-phosphate backbone or other equivalent covalent chemistry. Double and single-stranded DNAs and RNAs are typical examples of polynucleotides.

The term “polypeptide” or “protein” means a molecule that comprises at least two amino acid residues linked by a peptide bond to form a polypeptide. Small polypeptides of less than about 50 amino acids may be referred to as “peptides”.

“Valent” as used herein refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule.

The term “in combination with” as used herein means that two or more therapeutics can be administered to a subject together in a mixture, concurrently as single agents or sequentially as single agents in any order.

“Synergy”, “synergism” or “synergistic” mean more than the expected additive effect of a combination.

The term “heterologous” means that the polypeptide described has been derived from different cell types or different species and does not exist in nature.

The term “chimeric antigen receptor” or “CAR,” as used herein, refers to an heterologous cell surface receptor that is engineered to be expressed on an immune effector cell, such as a macrophage, and specifically bind an antigen, such as PSMA. CARs may be used as a therapy with adoptive cell transfer. Monocytes can be removed from a patient (blood, tumor or ascites fluid) and modified so that they express the receptors specific to a particular form of antigen, such as PSMA. In some embodiments, the CARs have been expressed with specificity to a tumor associated antigen, for example. CARs may also comprise an intracellular activation domain, a transmembrane domain and an extracellular domain comprising a tumor associated antigen binding region. In some embodiments, CARs comprise fusions of FN3 domains that bind to PSMA, such as those provided herein, fused or linked to a transmembrane domain and an intracellular domain. In some embodiments, a CAR can target cancers by redirecting a monocyte/macrophage expressing the CAR specific for tumor associated antigens, which can be done by utilizing a FN3 polypeptide that binds to PSMA.

### Compositions of Matter

In some embodiments, the present embodiments provide human PSMA binding FN3 domains and PSMA binding FN3 domains conjugated to toxins or detectable labels. The present invention provides polypeptides comprising a first FN3 domain and a second FN3 domain, wherein the first and second FN3 domains each bind to human prostate specific membrane antigen (PSMA). The present invention provides cells comprising a polypeptide that comprises at least one heterologous fibronectin type III (FN3) domain, wherein the at least one heterologous FN3 domain is on the surface of the cell and binds to human prostate specific membrane antigen (PSMA). The present invention provides polynucleotides encoding the FN3 domains of the invention or complementary nucleic acids thereof, vectors, host cells, and methods of making and using them.

### PSMA Binding Molecules

The present embodiments provide, in part, fibronectin type III (FN3) domains that bind specifically to human prostate specific membrane antigen (PSMA), optionally conjugated to a toxin or a detectable label. These molecules may be widely used in therapeutic and diagnostic applications. The present invention provides polynucleotides encoding the FN3 domains of the invention or complementary nucleic acids thereof, vectors, host cells, and methods of making and using them.

The FN3 domains of the invention bind PSMA with high affinity and are internalized into PSMA expressing cells, thereby providing an efficient way to deliver therapeutic drugs into tumor cells.

One embodiment of the invention an isolated FN3 domain that specifically binds human prostate specific membrane antigen (PSMA) of SEQ ID NO: 144.

In some embodiment of the invention described herein, the FN3 domain of the invention cross-reacts with Macaca Fascicularis PSMA of SEQ ID NO: 32 or with Pan troglodytes PSMA of SEQ ID NO: 33.

The FN3 domain of the invention may bind human, Macaca Fascicularis and/or Pan troglodytes PSMA with a dissociation constant (Ku) of less than about 1×10⁻⁷ M, for example less than about 1×10⁻⁸ M, less than about 1×10⁻⁹ M, less than about 1×10⁻¹⁰ M, less than about 1×10⁻¹¹ M, less than about 1×10⁻¹² M, or less than about 1×10⁻¹³ M as determined by surface plasmon resonance or the Kinexa method, as practiced by those of skill in the art. The measured affinity of a particular FN3 domain-antigen interaction can vary if measured under different conditions (e.g., osmolarity, pH). Thus, measurements of affinity and other antigen-binding parameters (e.g., K_{D}, Kₒₙ, K_{off}) are made with standardized solutions of protein scaffold and antigen, and a standardized buffer, such as the buffer described herein.

In some embodiments, the PSMA binding FN3 domains comprises an initiator methionine (Met) linked to the N-terminus of the molecule.

In some embodiments, the PSMA binding FN3 domains comprise a cysteine (Cys) linked to a C-terminus of the FN3 domain.

The addition of the N-terminal Met and/or the C-terminal Cys may facilitate expression and/or conjugation of half-life extending molecules.

Another embodiment of the invention is an isolated FN3 domain that specifically binds human PSMA, wherein the FN3 domain inhibits human PSMA enzymatic activity. PSMA enzymatic activity may be measured using standard methods. For example, hydrolysis of a detectable or labeled PSMA substrate of PSMA may be used. Exemplary PSMA substrates that may be used are N-Acetyl Aspartyl Glutamate (NAAG), folate polyglutamate, methotrexate tri-gamma glutamate, methotrexate di-gamma glutamate, pteroylpentaglutamate and derivatives thereof. The substrate may be labeled, for example, with a radioactive marker, chemiluminescent marker, enzymatic marker, chromogenic marker, or other detectable marker. Suitable methods for detecting PSMA activity are described, for example, in U.S. Pat. No. 5,981,209 or U.S. Pat. Publ. No. 2006/0009525. The isolated PSMA binding FN3 domain of the invention inhibits human PSMA enzymatic activity when the molecule inhibits human PSMA activity more than about 20%, 30%, 40%, 50%, 60%, 70%, 80%, 90%, or more when compared to a sample without the FN3 domain.

In some embodiments of the invention described herein, the isolated FN3 domain comprises the amino acid sequence of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169.

In some embodiments the FN3 domain comprises an amino acid sequence that is 89%, 90%, 91%, 92%, 93%, 94%, 95%, 96%, 97%, 98%, or 99%, identical to the amino acid sequence of the FN3 domains provided for herein.

In some embodiments of the invention described herein, the FN3 domain comprises an amino acid sequence that is 89%, 90%, 91%, 92%, 93%, 94%, 95%, 96%, 97%, 98%, or 99%, identical to the amino acid sequence of SEQ ID NO: 41. In some embodiments, the FN3 domain has the amino acid sequence of SEQ ID NO: 41.

In some embodiments of the invention described herein, the isolated FN3 domain comprises an amino acid sequence that has 1, 2, 3, 4, 5, 6, 7, 8, 9, 10 or 11 substitutions when compared to the amino acid sequence of SEQ ID NO: 41.

In some embodiments of the invention described herein, the isolated FN3 domain comprises an amino acid sequence that is 89%, 90%, 91%, 92%, 93%, 94%, 95%, 96%, 97%, 98%, or 99%, identical to the amino acid sequence of SEQ ID NO: 39. In some embodiments, the FN3 domain has the amino acid sequence of SEQ ID NO: 39.

In some embodiments of the invention described herein, the isolated FN3 domain comprises an amino acid sequence that has 1, 2, 3, 4, 5, 6, 7, 8, 9, 10 or 11 substitutions when compared to the amino acid sequence of SEQ ID NO: 39.

In some embodiments of the invention described herein, the isolated FN3 domain that specifically binds human PSMA comprises a cysteine residue in at least one residue position corresponding to residue positions 6, 11, 22, 25, 26, 52, 53, 61 of SEQ ID NO 1, or at a C-terminus. In some embodiments, the sequence that has the substitution is a sequence of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169. In some embodiments, a polypeptide of SEQ ID NO: 39 has the cysteine substitution. In some embodiments, a polypeptide of SEQ ID NO: 41 has the cysteine substitution. A non-limiting example method of determining which residues correspond to a position of 6, 11, 22, 25, 26, 52, 53, 61 of SEQ ID NO 1, the sequences provides herein, such as SEQ ID NO: 39 or 41, can be aligned with SEQ ID NO: 1. This can be done utilizing BlastP with the default parameters.

Substitutions resulting in introduction of cysteine into a protein sequence may be utilized to chemically conjugate small molecules such as cytotoxic agents, detectable labels, polyethylene glycol and/or nucleic acids to the FN3 domain using standard chemistry. In some embodiments, the nucleic acid molecule is an antisense molecule. In some embodiments, the nucleic acid molecule is a siRNA.

In some embodiments, the FN3 domain specifically binding human PSMA competes for binding to human PSMA with the FN3 domain of SEQ ID NO: 41.

In some embodiments, the FN3 domain specifically binding human PSMA competes for binding to human PSMA with the FN3 domain of SEQ ID NO: 39.

In some embodiments, the FN3 domain specifically binding human PSMA binds to the region KKSPSPEFSGMPRISK (SEQ ID NO: 159) and NWETNKF (SEQ ID NO: 160) of human PSMA.

The human PSMA epitope bound by the FN3 domain of the invention can include, for example, some or all of the residues within the amino acid sequences shown in SEQ ID NO: 159 or SEQ ID NO: 160. In some embodiments disclosed herein, the epitope bound by the FN3 domain of the invention comprises at least one amino acid in the region KKSPSPEFSGMPRISK (SEQ ID NO: 159) and NWETNKF (SEQ ID NO: 160) of human PSMA (SEQ ID NO: 144). In some embodiments disclosed herein, the epitope bound by the FN3 domain of the invention comprises at least two, three, four, five, six or seven amino acids in the region KKSPSPEFSGMPRISK (SEQ ID NO: 159) and at least two, three, four, five or six amino acids in the region NWETNKF (SEQ ID NO: 160) of human PSMA (SEQ ID NO: 144).

In some embodiments disclosed herein, the FN3 domain of the invention binds human PSMA at residues K499, K500, 5501, P502, P504, R511, K514, N540, W541, E542, N544, K545 and F546 (residue numbering according to SEQ ID NO: 144).

In some embodiments disclosed herein, the FN3 domain of the invention further binds human PSMA at residues R181, Y460, F488, K610 and/or I614.

The crystal structure of the FN3 domain P233FR9_H10 was solved in complex with cynoPSMA. As the contact residues between human and cyno PSMA are identical except for one residue, it is expected that P233FR9_H10 will bind human PSMA at the same epitope residues than what it binds cyno PSMA.

FN3 domains may be evaluated for their competition with a reference molecule for binding human PSMA using well known in vitro methods. In an exemplary method, CHO cells recombinantly expressing human PSMA may be incubated with unlabeled reference molecule for 15 min at 4° C., followed by incubation with an excess of fluorescently labeled test FN3 domain for 45 min at 4° C. After washing in PBS/BSA, fluorescence may be measured by flow cytometry using standard methods. In another exemplary method, extracellular portion of human PSMA may be coated on the surface of an ELISA plate. Excess of unlabeled reference molecule may be added for about 15 minutes and subsequently biotinylated test FN3 domains may be added. After washes in PBS/Tween, binding of the test biotinylated FN3 domain may be detected using horseradish peroxidase (HRP)-conjugated streptavidine and the signal detected using standard methods. It is readily apparent that in the competition assays, reference molecule may be labelled and the test FN3 domain unlabeled. The test FN3 domain competes with the reference molecule when the reference molecule inhibits binding of the test FN3 domain, or the test FN3 domain inhibits binding of the reference molecule by 20%, 30%, 40%, 50%, 60%, 70%, 80%, 85%, 90%, 95%, or 100%. The epitope of the test FN3 domain may further be defined for example by peptide mapping or hydrogen/deuterium protection assays using known methods, or by crystal structure determination. An exemplary reference FN3 domain is the domain comprising the amino acid sequence of SEQ ID NO: 41. An exemplary reference FN3 domain is the domain comprising the amino acid sequence of SEQ ID NO: 39.

FN3 domains binding to the same region on human PSMA as the FN3 domain of SEQ ID NO: 41 may be generated for example by immunizing mice with peptides having the amino acid sequences shown in SEQ ID NOs: 159 and 160 using standard methods and as described herein. FN3 domains may be further evaluated for example by assaying competition between the FN3 domain of SEQ ID NO: 41 and a test FN3 domain for binding to human PSMA using well known in vitro methods and as described above.

FN3 domains binding to the same region on human PSMA as the FN3 domain of SEQ ID NO: 39 may be generated for example by immunizing mice with peptides having the amino acid sequences shown in SEQ ID NOs: 159 and 160 using standard methods and as described herein. FN3 domains may be further evaluated for example by assaying competition between the FN3 domain of SEQ ID NO: 39 and a test FN3 domain for binding to human PSMA using well known in vitro methods and as described above.

In some embodiments, the isolated FN3 domain that specifically binds human PSMA of the invention is conjugated to a detectable label.

Detectable label includes compositions that when conjugated to the FN3 domain of the invention renders the latter detectable, via spectroscopic, photochemical, biochemical, immunochemical, or chemical means. Exemplary labels include radioactive isotopes, magnetic beads, metallic beads, colloidal particles, fluorescent dyes, electron-dense reagents, enzymes (for example, as commonly used in an ELISA), biotin, digoxigenin, or haptens. Specific radioactive labels include most common commercially available isotopes including, for example, ³H, ⁿC, ¹³C, ¹⁵N, ¹⁸F, ¹⁹F, ¹²³l, ¹²⁴l, ¹²⁵I, ¹³¹l, ⁸⁶Y, ⁸⁹Zr, ^{U1}ln, ^{94m}Tc, ^{99m}Tc, ⁶⁴Cu and ⁶⁸Ga. Suitable dyes include any commercially available dyes such as, for example, 5(6)-carboxyfluorescein, IRDye 680RD maleimide or IRDye 800CW, ruthenium polypyridyl dyes, and the like.

The FN3 domains that specifically binds human PSMA conjugated to a detectable label may be used as an imaging agent to evaluate tumor distribution, diagnosis for the presence of tumor cells and/or recurrence of tumor.

In some embodiments, the FN3 domains specifically binding human PSMA of the invention is conjugated to a cytotoxic agent.

In some embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

The FN3 domains specifically binding human PSMA conjugated to a cytotoxic agent may be used in the targeted delivery of the cytotoxic agent to PSMA expressing tumor cell, and intracellular accumulation therein, wherein systemic administration of the unconjugated cytotoxic agents may result in unacceptable levels of toxicity to normal cells.

In some embodiments, the cytotoxic agent is daunomycin, doxorubicin, methotrexate, vindesine, bacterial toxins such as diphtheria toxin, ricin, geldanamycin, maytansinoids or calicheamicin. The cytotoxic agent may elicit their cytotoxic and cytostatic effects by mechanisms including tubulin binding, DNA binding, or topoisomerase inhibition.

In some embodiments, the cytotoxic agent is an enzymatically active toxins such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, the cytotoxic agent is a radionuclide, such as ²¹²Bi, ¹³¹I, ¹³¹In, ⁹⁰Y, and ¹⁸⁶Re.

Conjugates of the FN3 domains of the invention and the cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, the cytotoxic agent is dolastatins or dolostatin peptidic analogs and derivatives, auristatin or monomethyl auristatin phenylalanine. Exemplary molecules are disclosed in U.S. Pat. Nos. 5,635,483 and 5,780,588. Dolastatins and auristatins have been shown to interfere with microtubule dynamics, GTP hydrolysis, and nuclear and cellular division and have anticancer and antifungal activity. The dolastatin or auristatin drug moiety may be attached to the FN3 domain containing molecule of the invention through the N (amino) terminus or the C (carboxyl) terminus of the peptidic drug moiety (WO 02/088172), or via any cysteine engineered into the FN3 domain.

In some embodiment, the FN3 domain specifically binding human PSMA is removed from the blood via renal clearance.

In some embodiments, the FN3 domain includes a signal sequence at the N-terminus or C-terminus of the polypeptide. In some embodiments, the signal sequence has an amino acid sequence of SEQ ID NO: 164.

In some embodiments, the FN3 domain includes a CD8 hinge sequence at the N-terminus or C-terminus of the polypeptide. In some embodiments, the CD8 hinge sequence has an amino acid sequence of SEQ ID NO: 165.

In some embodiments, the FN3 domain includes a signal sequence at the N-terminus and a CD8 hinge sequence at the C-terminus of the polypeptide. In some embodiments, the FN3 domain includes a CD8 hinge sequence at the N-terminus and a signal sequence at the C-terminus of the polypeptide. In some embodiments, the FN3 domain has an amino acid sequence of SEQ ID NO: 166, 167, or 172.

In some embodiments, the FN3 domain further comprises a linker between the FN3 domain and the CD8 hinge sequence. In some embodiments, the linker is a peptide linker. In some embodiments, the peptide linker is a glycine/serine or glycine/alanine linker. In some embodiments, the linker comprises an amino acid sequence of (GGGGS/A)n, wherein n is 1-5. In some embodiments, the linker comprises an amino acid sequence of SEQ ID NOs: 148, 149, 150, 151, 152, 153, 154, 142, 162, or 163.

In some embodiments, the FN3 domain has an amino acid sequence of SEQ ID NO: 170 or 171.

### Bi-Paratopic Molecules

Bi-paratopic molecules can be beneficial as a cancer treatment or for use as a detecting agent. Bi-paratopic molecules are capable of binding 2 epitopes simultaneously.

In some embodiments, a bi-paratopic molecule comprises two (2) FN3 domains that specifically bind human prostate specific membrane antigen (PSMA). In some embodiments, the FN3 domains are separated by a linker.

In some embodiments, each FN3 domain has an amino acid sequence, independently, selected from the group consisting of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169. In some embodiments, the linker has an amino acid sequence selected from the group consisting of SEQ ID NOs: (GS)₂, (SEQ ID NO: 148), (GGGS)₂ (SEQ ID NO: 149), (GGGGS)₅ (SEQ ID NO: 150), (AP)₂ (SEQ ID NO: 151), (AP)₅ (SEQ ID NO: 152), (AP)₁₀ (SEQ ID NO: 153), (AP)₂₀ (SEQ ID NO: 154), A(EAAAK)₅AAA (SEQ ID NO: 142), (GGGS)₄ (SEQ ID NO: 162), and (GGGS)₃ (SEQ ID NO: 163). The 2 FN3 domains may be linked to each other in a N-to C-direction.

In some embodiments, a bi-paratopic molecule comprises the 2 FN3 domains of SEQ ID NO: 39 and SEQ ID NO: 41. In some embodiments, the biparatopic molecule comprises one FN3 domain comprising the sequence of SEQ ID NO: 39 and a second FN3 domain selected from 35, 36, 37, 38, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169. In some embodiments, the biparatopic molecule comprises one FN3 domain comprising the sequence of SEQ ID NO: 41 and a second FN3 domain selected from 35, 36, 37, 38, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169.

In some embodiments, the FN3 domains are linked by a linker of SEQ ID NO 162.

In some embodiments, a bi-paratopic molecule comprises the amino acid sequence of SEQ ID NO: 161.

In some embodiments, the polypeptide has a formula of from the N-terminus to C-terminus of A1-L-A2, wherein A1 is a FN3 domain comprising a sequence of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169; wherein L is a peptide linker; and A2 is a FN3 domain comprising a sequence of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169.

In some embodiments, the peptide linker has an amino acid sequence selected from the group consisting of SEQ ID NOs: (GS)₂, (SEQ ID NO: 148), (GGGS)₂ (SEQ ID NO: 149), (GGGGS)₅ (SEQ ID NO: 150), (AP)₂ (SEQ ID NO: 151), (AP)₅ (SEQ ID NO: 152), (AP)₁₀ (SEQ ID NO: 153), (AP)₂₀ (SEQ ID NO: 154), A(EAAAK)₅AAA (SEQ ID NO: 142), (GGGS)₄ (SEQ ID NO: 162), and (GGGS)₃ (SEQ ID NO: 163). In some embodiments, the linker is a linker of SEQ ID NO: 162.

In some embodiments, A1 is SEQ ID NO: 39. In some embodiments, A1 is SEQ ID NO: 41. In some embodiments, A1 is SEQ ID NO: 39 and A2 is SEQ ID NO: 41. In some embodiments, A1 is SEQ ID NO: 41 and A2 is SEQ ID NO: 39.

In some embodiments, the polypeptide of formula A1-L-A2 comprises the sequence of SEQ ID NO: 161.

In some embodiments A1 and A2 are the same. In some embodiments, A1 and A2 are different.

Embodiments described herein are also directed to a cell comprising a polypeptide that comprises at least one heterologous fibronectin type III (FN3) domain, wherein the at least one heterologous FN3 domain is on the surface of the cell and binds to human prostate specific membrane antigen (PSMA). In some embodiments, the cell is a macrophage.

In some embodiments, the at least one heterologous fibronectin type III (FN3) domain is operably connected to an intracellular polypeptide. In some embodiments, the at least one heterologous fibronectin type III (FN3) domain is operably connected to the intracellular polypeptide through a transmembrane domain polypeptide. The intracellular polypeptide can comprise a protein sequence that transmits a signal and activates the cell when the FN3 domain binds to PSMA on the target cell. The transmembrane domain can be any domain that is used to create a transmembrane protein.

In some embodiments, the cell comprises a polypeptide that comprises at least one heterologous fibronectin type III (FN3) domain, wherein the at least one heterologous FN3 domain is on the surface of the cell and binds to human prostate specific membrane antigen (PSMA) present on the surface of a different cell. In some embodiments, the different cell is a prostate cell. In some embodiments, the different cell is a prostate cancer cell.

In some embodiments, the at least one heterologous FN3 domain that binds PSMA has an amino acid sequence selected from the group consisting of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, and 169. In some embodiments, the at least one heterologous FN3 domain that binds PSMA has an amino acid sequence of SEQ ID NO: 39. In some embodiments, the at least one heterologous FN3 domain that binds PSMA has an amino acid sequence of SEQ ID NO: 41.

In some embodiments, a cell comprises a polypeptide comprises a first heterologous FN3 domain and a second heterologous FN3 domain that bind to PSMA. In some embodiments, the first heterologous FN3 domain and the second heterologous FN3 domain bind to different epitopes on PSMA. In some embodiments, the different epitopes on PSMA do not overlap. In some embodiments, the first heterologous FN3 domain and the second heterologous FN3 domains are different.

In some embodiments, the first heterologous FN3 domain and the second heterologous FN3 domain each, independently, have an amino acid sequence selected from the group consisting of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, and 169.

In some embodiments, the first heterologous FN3 domain and the second heterologous FN3 domain of the polypeptide is comprised from N-terminus to C-terminus the first heterologous FN3 domain followed by the second heterologous FN3 domain.

In some embodiments, the first heterologous FN3 domain and the second heterologous FN3 domain of the polypeptide is comprised from N-terminus to C-terminus the second heterologous FN3 domain followed by the first heterologous FN3 domain.

In some embodiments, the first heterologous FN3 domain and the second heterologous FN3 domain of the polypeptide is comprised of the first heterologous FN3 domain having an amino acid sequence of SEQ ID NO: 39 and the second heterologous FN3 domain having an amino acid sequence of SEQ ID NO: 41.

In some embodiments, the first heterologous FN3 domain and the second heterologous FN3 domains are connected by a linker. In some embodiments, the linker is a peptide linker. In some embodiments, the peptide linker is a glycine/serine or glycine/alanine linker. In some embodiments, the linker comprises an amino acid sequence of (GGGGS/A)n, wherein n is 1-5. In some embodiments, the linker comprises an amino acid sequence of SEQ ID NOs: 148, 149, 150, 151, 152, 153, 154, 142, 162, or 163. In some embodiments, the linker comprises an amino acid sequence of SEQ ID NO: 162.

In some embodiments, the polypeptide has an amino acid sequence of SEQ ID NO: 161.

In some embodiments, the first heterologous FN3 domain and the second heterologous FN3 domain are the same. In some embodiments, both the first heterologous FN3 domain and the second heterologous FN3 domain have an amino acid sequence selected from the group consisting of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, and 169.

In some embodiments, the first heterologous FN3 domain and the second heterologous FN3 domain are connected by a linker. In some embodiments, the linker is a peptide linker. In some embodiments, the peptide linker is a glycine/serine or glycine/alanine linker. In some embodiments, the linker comprises an amino acid sequence of (GGGGS/A)n, wherein n is 1-5. In some embodiments, the linker comprises an amino acid sequence of SEQ ID NOs: 148, 149, 150, 151, 152, 153, 154, 142, 162, or 163.

Embodiments described herein are directed to a polypeptide comprising a first FN3 domain and a second FN3 domain, wherein the first FN3 domain and the second FN3 domain each bind to human prostate specific membrane antigen (PSMA). In some embodiments, polypeptide having the first FN3 domain and the second FN3 domain bind to different epitopes on PSMA. In some embodiments, the epitopes do not overlap.

In some embodiments, the first FN3 domain and the second FN3 domain are connected by a linker. In some embodiments, the linker is a peptide linker. In some embodiments, the peptide linker is a glycine/serine or glycine/alanine linker. In some embodiments, the linker comprises an amino acid sequence of (GGGGS/A)n, wherein n is 1-5. In some embodiments, the linker comprises an amino acid sequence of SEQ ID NOs: 148, 149, 150, 151, 152, 153, 154, 142, 162, or 163.

In some embodiments, the polypeptide comprises a first FN3 domain and a second FN3 domain that bind to PSMA. In some embodiments, the first FN3 domain and the second FN3 domain bind to different epitopes on PSMA. In some embodiments, the different epitopes on PSMA do not overlap. In some embodiments, the first FN3 domain and the second FN3 domains are different.

In some embodiments, the polypeptide comprises the first FN3 domain and the second FN3 domain each, independently, have an amino acid sequence selected from the group consisting of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, and 169.

In some embodiments, the first FN3 domain and the second FN3 domain of the polypeptide is comprised from N-terminus to C-terminus the first heterologous FN3 domain followed by the second heterologous FN3 domain.

In some embodiments, the first FN3 domain and the second FN3 domain of the polypeptide is comprised from N-terminus to C-terminus the second FN3 domain followed by the first FN3 domain.

In some embodiments, the first FN3 domain and the second FN3 domain of the polypeptide is comprised of the first FN3 domain having an amino acid sequence of SEQ ID NO: 39 and the second FN3 domain having an amino acid sequence of SEQ ID NO: 41.

In some embodiments, the polypeptide has an amino acid sequence of SEQ ID NO: 161.

In some embodiments, the first FN3 domain and the second FN3 domain are the same. In some embodiments, both the first FN3 domain and the second FN3 domain have an amino acid sequence selected from the group consisting of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, and 169.

In some embodiments, the first FN3 domain and the second FN3 domain are connected by a linker. In some embodiments, the linker is a peptide linker. In some embodiments, the peptide linker is a glycine/serine or glycine/alanine linker. In some embodiments, the linker comprises an amino acid sequence of (GGGGS/A)n, wherein n is 1-5. In some embodiments, the linker comprises an amino acid sequence of SEQ ID NOs: 148, 149, 150, 151, 152, 153, 154, 142, 162, or 163.

Embodiments described herein are directed to a cell comprising a polypeptide comprising a first FN3 domain and a second FN3 domain, wherein the first FN3 domain and the second FN3 domain each bind to human prostate specific membrane antigen (PSMA). In some embodiments, polypeptide having the first FN3 domain and the second FN3 domain bind to different epitopes on PSMA. In some embodiments, the epitopes do not overlap.

In some embodiments, the polypeptide comprises an intracellular domain operably connected to the first FN3 domain and the second FN3 domain.

In some embodiments, the intracellular domain is operably connected to the first FN3 domain and second FN3 domain by a transmembrane domain polypeptide.

In some embodiments, the first FN3 domain and the second FN3 domain are connected by a linker. In some embodiments, the linker is a peptide linker. In some embodiments, the peptide linker is a glycine/serine or glycine/alanine linker. In some embodiments, the linker comprises an amino acid sequence of (GGGGS/A)n, wherein n is 1-5. In some embodiments, the linker comprises an amino acid sequence of SEQ ID NOs: 148, 149, 150, 151, 152, 153, 154, 142, 162, or 163.

In some embodiments, the polypeptide comprises a first FN3 domain and a second FN3 domain that bind to PSMA. In some embodiments, the first FN3 domain and the second FN3 domain bind to different epitopes on PSMA. In some embodiments, the different epitopes on PSMA do not overlap. In some embodiments, the first FN3 domain and the second FN3 domains are different.

In some embodiments, the polypeptide comprises the first FN3 domain and the second FN3 domain each, independently, have an amino acid sequence selected from the group consisting of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, and 169.

In some embodiments, the first FN3 domain and the second FN3 domain of the polypeptide is comprised from N-terminus to C-terminus the first heterologous FN3 domain followed by the second heterologous FN3 domain.

In some embodiments, the first FN3 domain and the second FN3 domain of the polypeptide is comprised from N-terminus to C-terminus the second FN3 domain followed by the first FN3 domain.

In some embodiments, the first FN3 domain and the second FN3 domain of the polypeptide is comprised of the first FN3 domain having an amino acid sequence of SEQ ID NO: 39 and the second FN3 domain having an amino acid sequence of SEQ ID NO: 41.

In some embodiments, the polypeptide has an amino acid sequence of SEQ ID NO: 161.

In some embodiments, the first FN3 domain and the second FN3 domain are the same. In some embodiments, both the first FN3 domain and the second FN3 domain have an amino acid sequence selected from the group consisting of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, and 169.

In some embodiments, the first FN3 domain and the second FN3 domain are connected by a linker. In some embodiments, the linker is a peptide linker. In some embodiments, the peptide linker is a glycine/serine or glycine/alanine linker. In some embodiments, the linker comprises an amino acid sequence of (GGGGS/A)n, wherein n is 1-5. In some embodiments, the linker comprises an amino acid sequence of SEQ ID NOs: 148, 149, 150, 151, 152, 153, 154, 142, 162, or 163.

In some embodiments, the cell comprises a polypeptide having a formula of, from the N-terminus to C-terminus, of A1-L-A2, wherein A1 is a FN3 domain comprising a sequence of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169; wherein L is a peptide linker; and X2 is a FN3 domain comprising a sequence of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169.

In some embodiments, the peptide linker has an amino acid sequence selected from the group consisting of SEQ ID NOs: (GS)₂, (SEQ ID NO: 148), (GGGS)₂ (SEQ ID NO: 149), (GGGGS)₅ (SEQ ID NO: 150), (AP)₂ (SEQ ID NO: 151), (AP)₅ (SEQ ID NO: 152), (AP)₁₀ (SEQ ID NO: 153), (AP)₂₀ (SEQ ID NO: 154), A(EAAAK)₅AAA (SEQ ID NO: 142), (GGGS)₄ (SEQ ID NO: 162), and (GGGS)₃ (SEQ ID NO: 163). In some embodiments, the linker is a linker of SEQ ID NO: 162.

In some embodiments, A1 is SEQ ID NO: 39. In some embodiments, A1 is SEQ ID NO: 41. In some embodiments, A1 is SEQ ID NO: 39 and A2 is SEQ ID NO: 41. In some embodiments, A1 is SEQ ID NO: 41 and A2 is SEQ ID NO: 39.

In some embodiments, the polypeptide of formula A1-L-A2 comprises the sequence of SEQ ID NO: 161.

Embodiments described herein are directed to methods of treating prostate cancer in a subject, the method comprising administering a cell comprising a polypeptide that comprises at least one heterologous fibronectin type III (FN3) domain, wherein the at least one heterologous FN3 domain is on the surface of the cell and binds to human prostate specific membrane antigen (PSMA).

In some embodiments, macrophages are provided comprising a chimeric antigen receptor comprising at least one heterologous fibronectin type III (FN3) domain, wherein the at least one heterologous FN3 domain is on the surface of the cell and binds to human prostate specific membrane antigen (PSMA), is operably connected to a transmembrane domain and an intracellular domain of a stimulatory and/or co-stimulatory molecule.

In some embodiments, the at least one heterologous fibronectin type III (FN3) domain binds to a prostate cell expressing PSMA. In some embodiments, the at least one FN3 domain comprises the amino acid sequence of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169. In some embodiments the FN3 domain comprises an amino acid sequence that is 89%, 90%, 91%, 92%, 93%, 94%, 95%, 96%, 97%, 98%, or 99%, identical to the amino acid sequence of the FN3 domains provided for herein. In some embodiments the FN3 domain comprises an amino acid sequence that is 89%, 90%, 91%, 92%, 93%, 94%, 95%, 96%, 97%, 98%, or 99%, identical to the amino acid sequence of SEQ ID NO: 41. In some embodiments, the FN3 domain has the amino acid sequence of SEQ ID NO: 41. In some embodiments, the FN3 domain comprises an amino acid sequence that has 1, 2, 3, 4, 5, 6, 7, 8, 9, 10 or 11 substitutions when compared to the reference amino acid sequence provided for herein. In some embodiments, the reference sequence is SEQ ID NO: 39 or 41. In some embodiments, the FN3 domain comprises an amino acid sequence that is 89%, 90%, 91%, 92%, 93%, 94%, 95%, 96%, 97%, 98%, or 99%, identical to the amino acid sequence of SEQ ID NO: 39. In some embodiments, the FN3 domain has the amino acid sequence of SEQ ID NO: 39. In some embodiments, the FN3 domain comprises an amino acid sequence that has 1, 2, 3, 4, 5, 6, 7, 8, 9, 10 or 11 substitutions when compared to the reference amino acid sequence. In some embodiments, the sequence is SEQ ID NO: 39.

In some embodiments FN3 domain that specifically binds human PSMA comprises a cysteine residue in at least one residue position corresponding to residue positions 6, 11, 22, 25, 26, 52, 53, 61 of SEQ ID NO 1, or at a C-terminus. In some embodiments, the sequence that has the substitution is a sequence of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169. In some embodiments, a polypeptide of SEQ ID NO: 39 has the cysteine substitution. In some embodiments, a polypeptide of SEQ ID NO: 41 has the cysteine substitution.

In some embodiments, the prostate cell is a prostate cancer cell.

In some embodiments, the intracellular domain further comprises a hinge region. In some embodiments, the hinge domain comprises a CD8 hinge domain or an Ig hinge domain.

In some embodiments, the transmembrane domain comprises a CD8 transmembrane domain, a CD64 transmembrane domain, a CD16 transmembrane domain, a TLR1 transmembrane domain, a TLR2 transmembrane domain, a TLR4 transmembrane domain, a TLR5 transmembrane domain, or a TLR6 transmembrane domain.

In some embodiments, the intracellular domain comprises dual signaling domains. In some embodiments, the intracellular domain comprises a CD3 zeta intracellular domain, an FcεRI commonγ subunit intracellular domain, a Dectin-1 intracellular domain, a CD16 intracellular domain, a TLR1 intracellular domain, a TLR2 intracellular domain, a TLR4 intracellular domain, a TLR5 intracellular domain, or a TLR6 intracellular domain.

In some embodiments, the polypeptide comprises one or more spacer domains linking the transmembrane domain to the FN3 domain and/or the intracellular domain. In some embodiments, the linker comprises a sequence of SEQ ID NOs: (GS)₂, (SEQ ID NO: 148), (GGGS)₂ (SEQ ID NO: 149), (GGGGS)₅ (SEQ ID NO: 150), (AP)₂ (SEQ ID NO: 151), (AP)₅ (SEQ ID NO: 152), (AP)₁₀ (SEQ ID NO: 153), (AP)₂₀ (SEQ ID NO: 154), A(EAAAK)₅AAA (SEQ ID NO: 142), (GGGS)₄ (SEQ ID NO: 162), and (GGGS)₃ (SEQ ID NO: 163).

In some embodiments, the chimeric antigen receptor comprises a first heterologous FN3 domain and a second heterologous FN3 domain that bind to PSMA. In some embodiments, the first heterologous FN3 domain and the second heterologous FN3 domain bind to different epitopes on PSMA. In some embodiments, the different epitopes on PSMA do not overlap. In some embodiments, the first heterologous FN3 domain and the second FN3 domain are different. In some embodiments, the first heterologous FN3 domain and the second heterologous FN3 domain each, independently, have an amino acid sequence selected from the group consisting of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, and 169 or a sequence that is 89%, 90%, 91%, 92%, 93%, 94%, 95%, 96%, 97%, 98%, or 99%, identical to the same. In some embodiments, the FN3 domain has 1, 2, 3, 4, 5, 6, 7, 8, 9, 10, or 11 substitutions as compared to the reference sequences provided for herein. In some embodiments, each substitution is a single substitution.

In some embodiments, the chimeric antigen receptor comprises from N-terminus to C-terminus the first heterologous FN3 domain followed by the second heterologous FN3 domain. In some embodiments, the chimeric antigen receptor comprises from N-terminus to C-terminus the second heterologous FN3 domain followed by the first heterologous FN3 domain. Examples of the FN3 domains are provided for herein.

In some embodiments, the chimeric antigen receptor comprises a polypeptide having the formula of, from the N-terminus to C-terminus, of A1-L-A2, wherein A1 is a FN3 domain comprising a sequence of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169; wherein L is a peptide linker; and A2 is a FN3 domain comprising a sequence of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169.

In some embodiments, the peptide linker has an amino acid sequence selected from the group consisting of SEQ ID NOs: (GS)₂, (SEQ ID NO: 148), (GGGS)₂ (SEQ ID NO: 149), (GGGGS)₅ (SEQ ID NO: 150), (AP)₂ (SEQ ID NO: 151), (AP)₅ (SEQ ID NO: 152), (AP)₁₀ (SEQ ID NO: 153), (AP)₂₀ (SEQ ID NO: 154), A(EAAAK)₅AAA (SEQ ID NO: 142), (GGGS)₄ (SEQ ID NO: 162), and (GGGS)₃ (SEQ ID NO: 163). In some embodiments, the linker is a linker of SEQ ID NO: 162.

In some embodiments, A1 is SEQ ID NO: 39. In some embodiments, A1 is SEQ ID NO: 41. In some embodiments, X1 is SEQ ID NO: 39 and X2 is SEQ ID NO: 41. In some embodiments, A1 is SEQ ID NO: 41 and A2 is SEQ ID NO: 39.

In some embodiments, the polypeptide of formula A1-L-A2 comprises the sequence of SEQ ID NO: 161.

In some embodiments, A1 is a FN3 domain as provided for herein. In some embodiments, A2 is a FN3 domain as provided for herein. In some embodiments, A1 and A2 are different. In some embodiments, A1 and A2 are the same.

In some embodiments, the polypeptide of A1-L-A2 is operably connected to the transmembrane domain and the intracellular domain of a stimulatory and/or co-stimulatory molecule, such as provided for herein.

Also provided for herein are chimeric antigen receptors comprising a FN3 domain as provided for herein. In some embodiments, the chimeric antigen receptor comprises a polypeptide having the formula of A1-L-A2 as provided for herein. In some embodiments, the polypeptide of A1-L-A2 is operably connected to the transmembrane domain and the intracellular domain of a stimulatory and/or co-stimulatory molecule, such as provided for herein.

Embodiments described herein are directed to methods of treating prostate cancer in a subject, the method comprising administering a cell comprising a polypeptide comprising a first FN3 domain and a second FN3 domain, wherein the first FN3 domain and the second FN3 domain each bind to human prostate specific membrane antigen (PSMA).

FN3 Domain Expressing Cells

A type of treatment that has been shown to be beneficial in the treatment of cancer is the use of a cell which expresses a particular protein with the ability to target and bind a cancer cell which is expressing an antigen. In some embodiments, the cell is a macrophage. In some embodiments, the cell expresses a chimeric antigen receptor. In some embodiments, the cell is a macrophage. In some embodiments, the chimeric antigen receptor is as provided for herein.

In some embodiments, a cell comprises a fibronectin type III (FN3) domain on its surface wherein the cell targets and binds a cell expressing a human prostate specific membrane antigen (PSMA-expressing cell). In some embodiments, the polypeptide comprising the FN3 domain is a FN3 domain as provided for herein.

In certain embodiments, the FN3 domain that specifically binds PSMA has an amino acid sequence of SEQ ID NO: 39.

In certain embodiments, the FN3 domain that specifically binds PSMA has an amino acid sequence of SEQ ID NO: 41.

In certain embodiments, the FN3 domain that specifically binds PSMA has an amino acid sequence of SEQ ID NO: 161.

In certain embodiments, the FN3 domain that specifically binds PSMA has an amino acid sequence of SEQ ID NO: 166.

In certain embodiments, the FN3 domain that specifically binds PSMA has an amino acid sequence of SEQ ID NO: 167.

The FN3 domains provides for herein can be substituted with the other FN3 domains described herein, including the variants comprising the substitutions or % identity as provided for herein.

In some embodiments, a macrophage is provided that comprises a chimeric antigen receptor comprising at least one heterologous fibronectin type III (FN3) domain, wherein the at least one heterologous FN3 domain is on the surface of the cell and binds to human prostate specific membrane antigen (PSMA), operably connected to a transmembrane domain and an intracellular domain of a stimulatory and/or co-stimulatory molecule. In some embodiments, the at least one heterologous fibronectin type III (FN3) domain binds to a prostate cell expressing PSMA. In some embodiments, the prostate cell is a prostate cancer cell. In some embodiments, the CAR comprises two FN3 domains, such as provided for herein. In some embodiments, the polypeptide comprising the two FN3 domains is a polypeptide having the formula of A1-X-A2 as provided for herein.

With respect to the transmembrane domain, the CAR can be designed to comprise a transmembrane domain that connects the antigen binding domain (the FN3 domain) of the CAR to the intracellular domain. In some embodiments, the transmembrane domain is naturally associated with one or more of the domains in the CAR. In some embodiments, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. In some embodiments, the transmembrane domain may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9. In some embodiments, a variety of human hinges can be employed as well including the human Ig (immunoglobulin) hinge.

In some embodiments, the transmembrane domain may be synthetic. In some embodiments, the synthetic transmembrane domain can comprise predominantly hydrophobic residues such as leucine and valine. In some embodiments, a triplet of phenylalanine, tryptophan and valine will be found at each end of a synthetic transmembrane domain.

The intracellular domain or otherwise, the cytoplasmic domain of the CAR, includes a similar or the same intracellular domain as the chimeric intracellular signaling molecule described elsewhere herein, and is responsible for activation of the cell in which the CAR is expressed. In some embodiments, the intracellular domain of the CAR includes a domain responsible for signal activation and/or transduction.

Examples of an intracellular domain for use in the invention include, but are not limited to, the cytoplasmic portion of a surface receptor, co-stimulatory molecule, and any molecule that acts in concert to initiate signal transduction in the macrophage, as well as any derivative or variant of these elements and any synthetic sequence that has the same functional capability.

Examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

In some embodiments, the intracellular domain of the CAR comprises dual signaling domains, such as 41BB, CD28, ICOS, TLR1, TLR2, TLR3, TLR4, TLR5, TLR6, TLR7, TLR8, TLR9, TLR10, TLR11, CD116 receptor beta chain, CSF1-R, LRP1/CD91, SR-A1, SR-A2, MARCO, SR-CL1, SR-CL2, SR-C, SR-E, CR1, CR3, CR4, dectin 1, DEC-205, DC-SIGN, CD14, CD36, LOX-1, CD11b, together with any of the signaling domains listed in the above paragraph in any combination. In another embodiment, the intracellular domain of the CAR includes any portion of one or more co-stimulatory molecules, such as at least one signaling domain from CD3, Fc epsilon RI gamma chain, any derivative or variant thereof, any synthetic sequence thereof that has the same functional capability, and any combination thereof.

In some embodiments, between the antigen binding domain (e.g. the FN3 domain) and the transmembrane domain of the CAR, or between the intracellular domain and the transmembrane domain of the CAR, a spacer domain may be incorporated. As used herein, the term “spacer domain” generally means any oligo- or polypeptide that functions to link the transmembrane domain to, either the antigen binding domain or, the intracellular domain in the polypeptide chain. In some embodiments, the spacer domain may comprise up to 300 amino acids, 10 to 100 amino acids, or 25 to 50 amino acids. In some embodiments, a short oligo- or polypeptide linker, such as, but not limited to, from 2 and 10 amino acids in length may form the linkage between the transmembrane domain and the intracellular domain of the CAR. An example of a linker includes a glycine-serine doublet.

Isolating macrophages and incorporating a chimeric antigen receptor can be done, for example, as described in U.S. Publication No. 2020/0247870, which is hereby incorporated by reference in its entirety. U.S. Publication No. 2020/0247870 also describes how to expand and use such cells in therapy. In some embodiments, the macrophages are used to treat prostate cancer. f

Isolation of PSMA Binding FN3 Domains from a Library Based on Tencon Sequence

Tencon (SEQ ID NO: 1) is a non-naturally occurring fibronectin type III (FN3) domain designed from a consensus sequence of fifteen FN3 domains from human tenascin-C (U.S. Pat. Publ. No. 2010/0216708). The crystal structure of Tencon shows six surface-exposed loops that connect seven beta-strands as is characteristic to the FN3 domains, the beta-strands referred to as A, B, C, D, E, F, and G, and the loops referred to as AB, BC, CD, DE, EF, and FG loops (U.S. Pat. No. 6,673,901). These loops, or selected residues within each loop, may be randomized in order to construct libraries of fibronectin type III (FN3) domains that may be used to select novel molecules that bind PSMA. Table 1 shows positions and sequences of each loop and beta-strand in Tencon (SEQ ID NO: 1).

Library designed based on Tencon sequence may thus have randomized FG loop, or randomized BC and FG loops, such as libraries TCL1 or TCL2 as described below. The Tencon BC loop is 7 amino acids long, thus 1, 2, 3, 4, 5, 6 or 7 amino acids may be randomized in the library diversified at the BC loop and designed based on Tencon sequence. The Tencon FG loop is 7 amino acids long, thus 1, 2, 3, 4, 5, 6 or 7 amino acids may be randomized in the library diversified at the FG loop and designed based on Tencon sequence. Further diversity at loops in the Tencon libraries may be achieved by insertion and/or deletions of residues at loops. For example, the FG and/or BC loops may be extended by 1-22 amino acids, or decreased by 1-3 amino acids. The FG loop in Tencon is 7 amino acids long, whereas the corresponding loop in antibody heavy chains ranges from 4-28 residues. To provide maximum diversity, the FG loop may be diversified in sequence as well as in length to correspond to the antibody CDR3 length range of 4-28 residues. For example, the FG loop can further be diversified in length by extending the loop by additional 1, 2, 3, 4 or 5 amino acids.

Library designed based on Tencon sequence may also have randomized alternative surfaces that form on a side of the FN3 domain and comprise two or more beta strands, and at least one loop. One such alternative surface is formed by amino acids in the C and the F beta-strands and the CD and the FG loops (a C-CD-F-FG surface). A library design based on Tencon alternative C-CD-F-FG surface is is described in U.S. Pat. Publ. No. US2013/0226834. Library designed based on Tencon sequence also includes libraries designed based on Tencon variants, such as Tencon variants having substitutions at residues positions 11, 14, 17, 37, 46, 73, or 86 (residue numbering corresponding to SEQ ID NO: 1), and which variants display improve thermal stability. Exemplary Tencon variants are described in US Pat. Publ. No. 2011/0274623, and include Tencon27 (SEQ ID NO: 4) having substitutions E11R, L17A, N46V and E86I when compared to Tencon of SEQ ID NO: 1.

TABLE 1

| | Tencon |
|---|---|
| FN3 domain | (SEQ ID NO: 1) |
| A strand | 1-12 |
| AB loop | 13-16 |
| B strand | 17-21 |
| BC loop | 22-28 |
| C strand | 29-37 |
| CD loop | 38-43 |
| D strand | 44-50 |
| DE loop | 51-54 |
| E strand | 55-59 |
| EF loop | 60-64 |
| F strand | 65-74 |
| FG loop | 75-81 |
| G strand | 82-89 |

Tencon and other FN3 sequence based libraries may be randomized at chosen residue positions using a random or defined set of amino acids. For example, variants in the library having random substitutions may be generated using NNK codons, which encode all 20 naturally occurring amino acids. In other diversification schemes, DVK codons may be used to encode amino acids Ala, Trp, Tyr, Lys, Thr, Asn, Lys, Ser, Arg, Asp, Glu, Gly, and Cys. Alternatively, NNS codons may be used to give rise to all 20 amino acid residues and simultaneously reducing the frequency of stop codons. Libraries of FN3 domains with biased amino acid distribution at positions to be diversified may be synthesized for example using Slonomics® technology (http:_//www_sloning_com). This technology uses a library of pre-made double stranded triplets that act as universal building blocks sufficient for thousands of gene synthesis processes. The triplet library represents all possible sequence combinations necessary to build any desired DNA molecule. The codon designations are according to the well-known IUB code.

The FN3 domains specifically binding human PSMA of the invention may be isolated by producing the FN3 library such as the Tencon library using cis display to ligate DNA fragments encoding the scaffold proteins to a DNA fragment encoding RepA to generate a pool of protein-DNA complexes formed after in vitro translation wherein each protein is stably associated with the DNA that encodes it (U.S. Pat. No. 7,842,476), and assaying the library for specific binding to PSMA by any method known in the art and described in the Example Exemplary well known methods which can be used are ELISA, sandwich immunoassays, and competitive and non-competitive assays. The identified FN3 domains specifically binding PSMA are further characterized for their inhibition of PSMA activity, internalization, stability, and other desired characteristics.

The FN3 domains specifically binding human PSMA of the invention may be generated using any FN3 domain as a template to generate a library and screening the library for molecules specifically binding human PSMA using methods provided within. Exemplar FN3 domains that may be used are the 3rd FN3 domain of tenascin C (TN3) (SEQ ID NO: 145), Fibcon (SEQ ID NO: 146), and the 10^{th} FN3 domain of fibronectin (FN10) (SEQ ID NO: 147). Standard cloning and expression techniques are used to clone the libraries into a vector or synthesize double stranded cDNA cassettes of the library, to express, or to translate the libraries in vitro. For example ribosome display, mRNA display, or other cell-free systems (U.S. Pat. No. 5,643,768) can be used. The libraries of the FN3 domain variants may be expressed as fusion proteins displayed on the surface for example of any suitable bacteriophage. Methods for displaying fusion polypeptides on the surface of a bacteriophage are well known (U.S. Pat. Publ. No. 2011/0118144; Int. Pat. Publ. No. WO2009/085462; U.S. Pat. Nos. 6,969,108; 6,172,197; 5,223,409; 6,582,915; 6,472,147).

In some embodiments of the invention described herein, the FN3 domain specifically binding human PSMA is based on Tencon sequence of SEQ ID NO: 1 or Tencon27 sequence of SEQ ID NO: 4, the SEQ ID NO: 1 or the SEQ ID NO: 4, optionally having substitutions at residues positions 11, 14, 17, 37, 46, 73, and/or 86.

The FN3 domains specifically binding human PSMA of the invention may be modified to improve their properties such as improve thermal stability and reversibility of thermal folding and unfolding. Several methods have been applied to increase the apparent thermal stability of proteins and enzymes, including rational design based on comparison to highly similar thermostable sequences, design of stabilizing disulfide bridges, mutations to increase alpha-helix propensity, engineering of salt bridges, alteration of the surface charge of the protein, directed evolution, and composition of consensus sequences. High thermal stability may increase the yield of the expressed protein, improve solubility or activity, decrease immunogenicity, and minimize the need of a cold chain in manufacturing. Residues that may be substituted to improve thermal stability of Tencon (SEQ ID NO: 1) are residue positions 11, 14, 17, 37, 46, 73, or 86, and are described in US Pat. Publ. No. 2011/0274623. Substitutions corresponding to these residues may be incorporated to the FN3 domain containing molecules of the invention.

Measurement of protein stability and protein lability can be viewed as the same or different aspects of protein integrity. Proteins are sensitive or “labile” to denaturation caused by heat, by ultraviolet or ionizing radiation, changes in the ambient osmolarity and pH if in liquid solution, mechanical shear force imposed by small pore-size filtration, ultraviolet radiation, ionizing radiation, such as by gamma irradiation, chemical or heat dehydration, or any other action or force that may cause protein structure disruption. The stability of the molecule can be determined using standard methods. For example, the stability of a molecule can be determined by measuring the thermal melting (“Tₘ”) temperature, the temperature in ° Celsius (° C.) at which half of the molecules become unfolded, using standard methods. Typically, the higher the Tₘ, the more stable the molecule. In addition to heat, the chemical environment also changes the ability of the protein to maintain a particular three dimensional structure.

In one embodiment, the FN3 domain specifically binding human PSMA of the invention may exhibit increased stability by at least 5%, 10%, 15%, 20%, 25%, 30%, 35%, 40%, 45%, 50%, 55%, 60%, 65%, 70%, 75%, 80%, 85%, 90%, or 95% or more compared to the same domain prior to engineering measured by the increase in the Tₘ.

Chemical denaturation can likewise be measured by a variety of methods. Chemical denaturants include guanidinium hydrochloride, guanidinium thiocyanate, urea, acetone, organic solvents (DMF, benzene, acetonitrile), salts (ammonium sulfate, lithium bromide, lithium chloride, sodium bromide, calcium chloride, sodium chloride); reducing agents (e.g. dithiothreitol, beta-mercaptoethanol, dinitrothiobenzene, and hydrides, such as sodium borohydride), non-ionic and ionic detergents, acids (e.g. hydrochloric acid (HCl), acetic acid (CH₃COOH), halogenated acetic acids), hydrophobic molecules (e.g. phosopholipids), and targeted denaturants. Quantitation of the extent of denaturation can rely on loss of a functional property, such as ability to bind a target molecule, or by physiochemical properties, such as tendency to aggregation, exposure of formerly solvent inaccessible residues, or disruption or formation of disulfide bonds.

The FN3 domain of the invention may be generated as monomers, dimers, or multimers, for example, as a means to increase the valency and thus the avidity of target molecule binding, or to generate bi- or multispecific scaffolds simultaneously binding two or more different target molecules. The dimers and multimers may be generated by linking monospecific, bi- or multispecific protein scaffolds, for example, by the inclusion of an amino acid linker, for example a linker containing poly-glycine, glycine and serine, or alanine and proline. Exemplary linker include (GS)₂, (SEQ ID NO: 148), (GGGS)₂ (SEQ ID NO: 149), (GGGGS)₅ (SEQ ID NO: 150), (AP)₂ (SEQ ID NO: 151), (AP)₅ (SEQ ID NO: 152), (AP)₁₀ (SEQ ID NO: 153), (AP)₂₀ (SEQ ID NO: 154), A(EAAAK)₅AAA (SEQ ID NO: 142), (GGGS)₄ (SEQ ID NO: 162), and (GGGS)₃ (SEQ ID NO: 163). The dimers and multimers may be linked to each other in a N-to C-direction. The use of naturally occurring as well as artificial peptide linkers to connect polypeptides into novel linked fusion polypeptides is well known in the literature (U.S. Pat. No. 5,856,456).

### Half-Life Extending Moieties

The FN3 domain specifically binding human PSMA of the invention may incorporate other subunits for example via covalent interaction. In one aspect of the invention, the FN3 domain of the invention further comprises a half-life extending moiety. Exemplary half-life extending moieties are albumin, albumin variants, albumin-binding proteins and/or domains, transferrin and fragments and analogues thereof, and Fc regions. An exemplary albumin variant is shown in SEQ ID NO: 155 Amino acid sequences of the human Fc regions are well known, and include IgG1, IgG2, IgG3, IgG4, IgM, IgA and IgE Fc regions.

All or a portion of an antibody constant region may be attached to the FN3 domain of the invention to impart antibody-like properties, especially those properties associated with the Fc region, such as Fc effector functions such as C1q binding, complement dependent cytotoxicity (CDC), Fc receptor binding, antibody-dependent cell-mediated cytotoxicity (ADCC), phagocytosis, down regulation of cell surface receptors (e.g., B cell receptor; BCR), and may be further modified by modifying residues in the Fc responsible for these activities.

Additional moieties may be incorporated into the FN3 domain of the invention such as polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties may be direct fusions with the protein scaffold coding sequences and may be generated by standard cloning and expression techniques. Alternatively, well known chemical coupling methods may be used to attach the moieties to recombinantly produced molecules of the invention.

A pegyl moiety may for example be added to the FN3 domain of the invention by incorporating a cysteine residue to the C-terminus of the molecule, or engineering cysteines into residue positions that face away from the human PSMA binding face of the molecule, and attaching a pegyl group to the cysteine using well known methods.

FN3 domain of the invention incorporating additional moieties may be compared for functionality by several well-known assays. For example, altered properties due to incorporation of Fc domains and/or Fc domain variants may be assayed in Fc receptor binding assays using soluble forms of the receptors, such as the FcγRI, FcγRII, FcγRIII or FcRn receptors, or using well known cell-based assays measuring for example ADCC or CDC, or evaluating pharmacokinetic properties of the molecules of the invention in in vivo models.

### Polynucleotides, Vectors, Host Cells

The invention provides for nucleic acids encoding the FN3 domains specifically binding human PSMA of the invention as isolated polynucleotides or as portions of expression vectors or as portions of linear DNA sequences, including linear DNA sequences used for in vitro transcription/translation, vectors compatible with prokaryotic, eukaryotic or filamentous phage expression, secretion and/or display of the compositions or directed mutagens thereof. Certain exemplary polynucleotides are disclosed herein, however, other polynucleotides which, given the degeneracy of the genetic code or codon preferences in a given expression system, encode the FN3 domains of the invention are also within the scope of the invention.

One embodiment of the invention is an isolated polynucleotide encoding the FN3 domain specifically binding human PSMA comprising the amino acid sequence of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, or 169.

One embodiment of the invention is an isolated polynucleotide comprising the polynucleotide sequence of SEQ ID NOs: 156, 157, 158 or 159.

The polynucleotides of the invention may be produced by chemical synthesis such as solid phase polynucleotide synthesis on an automated polynucleotide synthesizer and assembled into complete single or double stranded molecules. Alternatively, the polynucleotides of the invention may be produced by other techniques such a PCR followed by routine cloning. Techniques for producing or obtaining polynucleotides of a given known sequence are well known in the art.

The polynucleotides of the invention may comprise at least one non-coding sequence, such as a promoter or enhancer sequence, intron, polyadenylation signal, a cis sequence facilitating RepA binding, and the like. The polynucleotide sequences may also comprise additional sequences encoding additional amino acids that encode for example a marker or a tag sequence such as a histidine tag or an HA tag to facilitate purification or detection of the protein, a signal sequence, a fusion protein partner such as RepA, Fc or bacteriophage coat protein such as pIX or pIII.

Another embodiment of the invention is a vector comprising at least one polynucleotide of the invention. Such vectors may be plasmid vectors, viral vectors, vectors for baculovirus expression, transposon based vectors or any other vector suitable for introduction of the polynucleotides of the invention into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

Another embodiment of the invention is a host cell comprising the vector of the invention. The FN3 domain specifically binding human PSMA of the invention may be optionally produced by a cell line, a mixed cell line, an immortalized cell or clonal population of immortalized cells, as well known in the art.

The host cell chosen for expression may be of mammalian origin or may be selected from COS-1, COS-7, HEK293, BHK21, CHO, BSC-1, He G2, SP2/0, HeLa, myeloma, lymphoma, yeast, insect or plant cells, or any derivative, immortalized or transformed cell thereof. Alternatively, the host cell may be selected from a species or organism incapable of glycosylating polypeptides, e.g. a prokaryotic cell or organism, such as BL21, BL21(DE3), BL21-GOLD(DE3), XL1-Blue, JM109, HMS174, HMS174(DE3), and any of the natural or engineered E. coli spp, Klebsiella spp., or Pseudomonas spp strains.

Another embodiment of the invention is a method of producing the isolated FN3 domain specifically binding human PSMA of the invention, comprising culturing the isolated host cell of the invention under conditions such that the isolated FN3 domain specifically binding human PSMA is expressed, and purifying the FN3 domain.

The FN3 domain specifically binding human PSMA may be purified from recombinant cell cultures by well-known methods, for example by protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography, or high performance liquid chromatography (HPLC).

### Uses of Human PSMA Binding FN3 Domains of the Invention

The FN3 domains specifically binding human PSMA of the invention may be used to diagnose, monitor, modulate, treat, alleviate, help prevent the incidence of, or reduce the symptoms of human disease or specific pathologies in cells, tissues, organs, fluid, or, generally, a host. The methods of the invention may be used to treat an animal patient belonging to any classification. Examples of such animals include mammals such as humans, rodents, dogs, cats and farm animals.

One embodiment of the invention is a method of treating a subject having cancer characterized by overexpression of PSMA, comprising administering to the subject a FN3 domain specifically binding human PSMA of the invention conjugated to a cytotoxic agent for a time sufficient to treat the subject.

In some embodiments, the cancer is prostate cancer, colorectal cancer, gastric cancer, clear cell renal carcinoma, bladder cancer, lung cancer or kidney cancer.

In some embodiments, the cancer is solid tumor.

In some embodiments, the cancer is a prostate disorder such as, for example, prostate cancer or benign prostatic hyperplasia (BPH).

In some embodiments, the cancer is prostate cancer.

In some embodiments, the cancer is colorectal cancer.

In some embodiments, the cancer is gastric cancer.

In some embodiments, the cancer is clear cell renal carcinoma.

In some embodiments, the cancer is bladder cancer.

In some embodiments, the cancer is kidney cancer.

In some embodiments, the cancer is a neovascular disorder such as, for example, a cancer characterized by solid tumor growth. Exemplary cancers with tumor vasculatures characterized by PSMA overexpression and amenable to treatment in accordance with the present invention include, for example, clear cell renal carcinoma (CCRCC), colorectal cancer, breast cancer, bladder cancer, lung cancer, and pancreatic cancer.

One embodiment of the invention is a method of treating a subject having prostate cancer characterized by overexpression of PSMA, comprising administering to the subject the FN3 domain specifically binding human PSMA of the invention conjugated of a cytotoxic agent for a time sufficient to treat the subject.

Subjects for administration of the FN3 domain specifically binding human PSMA of the invention as described herein include patients at high risk for developing a particular disorder characterized by PSMA overexpression as well as patients presenting with an existing such disorder. Typically, the subject has been diagnosed as having the disorder for which treatment is sought. Further, subjects can be monitored during the course of treatment for any change in the disorder (e.g., for an increase or decrease in clinical symptoms of the disorder).

In prophylactic applications, pharmaceutical compositions or medicants are administered to a patient susceptible to, or otherwise at risk of, a particular disorder in an amount sufficient to eliminate or reduce the risk or delay the onset of the disorder. In therapeutic applications, compositions or medicants are administered to a patient suspected of, or already suffering from such a disorder in an amount sufficient to cure, or at least partially arrest, the symptoms of the disorder and its complications. An amount adequate to accomplish this is referred to as a therapeutically effective dose or amount. In both prophylactic and therapeutic regimes, agents are usually administered in several dosages until a sufficient response (e.g., inhibition of inappropriate angiogenesis activity) has been achieved. Typically, the response is monitored and repeated dosages are given if the desired response starts to fade.

To identify subject patients for treatment according to the methods of the invention, accepted screening methods may be employed to determine risk factors associated with specific disorders or to determine the status of an existing disorder identified in a subject. Such methods can include, for example, determining whether an individual has relatives who have been diagnosed with a particular disorder. Screening methods may also include, for example, conventional work-ups to determine familial status for a particular disorder known to have a heritable component. For example, various cancers are also known to have certain inheritable components. Inheritable components of cancers include, for example, mutations in multiple genes that are transforming (e.g., Ras, Raf, EGFR, cMet, and others), the presence or absence of certain HLA and killer inhibitory receptor (KIR) molecules, or mechanisms by which cancer cells are able to modulate immune suppression of cells like NK cells and T cells, either directly or indirectly. Toward this end, nucleotide probes can be routinely employed to identify individuals carrying genetic markers associated with a particular disorder of interest. In addition, a wide variety of immunological methods are known in the art that are useful to identify markers for specific disorder. For example, various ELISA immunoassay methods are available and well-known in the art that employ monoclonal antibody probes to detect antigens associated with specific tumors. Screening can be implemented as indicated by known patient symptomology, age factors, related risk factors, etc. These methods allow the clinician to routinely select patients in need of the methods described herein for treatment. In accordance with these methods, targeting pathological, PSMA-expressing cells can be implemented as an independent treatment program or as a follow-up, adjunct, or coordinate treatment regimen to other treatments.

In some methods described herein, the FN3 domains specifically binding human PSMA of the invention conjugated to a cytotoxic agent may be used to treat a subject with prostate cancer in combination with a second therapeutic.

In some methods described herein, the FN3 domains specifically binding human PSMA of the invention conjugated to a cytotoxic agent may be used to treat a subject who is resistant or has acquired resistance to a treatment with a second therapeutic.

The second therapeutic may be an approved drug for the treatment of prostate cancer, such as Abiraterone Acetate (Zytiga), Bicalutamide, Cabazitaxel, Casodex (Bicalutamide), Degarelix, Docetaxel, Enzalutamide, Goserelin Acetate, Jevtana (Cabazitaxel), Leuprolide Acetate, Lupron (Leuprolide Acetate), Lupron Depot (Leuprolide Acetate), Lupron Depot-3 Month (Leuprolide Acetate), Lupron Depot-4 Month (Leuprolide Acetate), Lupron Depot-Ped (Leuprolide Acetate), Mitoxantrone Hydrochloride, Prednisone, Provenge (Sipuleucel-T), Radium 223 Dichloride, Sipuleucel-T, Taxotere (Docetaxel), Viadur (Leuprolide Acetate), Xofigo (Radium 223 Dichloride), Xtandi (Enzalutamide) or Zoladex (Goserelin Acetate) (source: National Cancer Institute).

Various qualitative and/or quantitative methods may be used to determine if a subject is resistant, has developed or is susceptible to developing a resistance to treatment. Symptoms that may be associated with resistance include, for example, a decline or plateau of the well-being of the patient, an increase in the size of a tumor, arrested or slowed decline in growth of a tumor, and/or the spread of cancerous cells in the body from one location to other organs, tissues or cells. Re-establishment or worsening of various symptoms associated with cancer may also be an indication that a subject has developed or is susceptible to developing resistance to treatment, such as anorexia, cognitive dysfunction, depression, dyspnea, fatigue, hormonal disturbances, neutropenia, pain, peripheral neuropathy, and sexual dysfunction. The symptoms associated with cancer may vary according to the type of cancer. For example, symptoms associated with prostate cancer may include trouble passing or frequent urge to pass urine, painful urination, blood in the urine or semen, nagging pain in the pelvis, back and/or hips. Symptoms associated with lung cancer may include persistent cough, coughing up blood, shortness of breath, wheezing chest pain, loss of appetite, losing weight without trying and fatigue. One skilled in oncology may readily identify symptoms associated with a particular cancer type.

The terms “treat” or “treatment” refers to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “therapeutically effective amount” refers to an amount effective, at dosages and for periods of time necessary, to achieve a desired therapeutic result. A therapeutically effective amount of the PSMA binding FN3 domain of the invention may vary according to factors such as the disease state, age, sex, and weight of the individual, and the ability of the PSMA binding FN3 domain of the invention to elicit a desired response in the individual. Exemplary indicators of an effective PSMA binding FN3 domain that may decline or abate in association with resistance include, for example, improved well-being of the patient, decrease or shrinkage of the size of a tumor, arrested or slowed growth of a tumor, and/or absence of metastasis of cancer cells to other locations in the body.

### Administration/Pharmaceutical Compositions

The invention provides for pharmaceutical compositions of the FN3 domains specifically binding human PSMA, optionally conjugated to a second molecule of the invention and a pharmaceutically acceptable carrier. For therapeutic use, the FN3 domains of the invention may be prepared as pharmaceutical compositions containing an effective amount of the domain or molecule as an active ingredient in a pharmaceutically acceptable carrier. The term “carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules of the invention in such pharmaceutical formulation can vary widely, i.e., from less than about 0.5%, usually at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin.

The mode of administration for therapeutic use of the FN3 domains of the invention may be any suitable route that delivers the agent to the host, such as parenteral administration, e.g., intradermal, intramuscular, intraperitoneal, intravenous or subcutaneous, pulmonary; transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

Thus, a pharmaceutical composition of the invention for intramuscular injection could be prepared to contain 1 ml sterile buffered water, and between about 1 ng to about 100 mg, e.g. about 50 ng to about 30 mg or more preferably, about 5 mg to about 25 mg, of the FN3 domain of the invention.

The FN3 domains of the invention may be administered to a patient by any suitable route, for example parentally by intravenous (IV) infusion or bolus injection, intramuscularly or subcutaneously or intraperitoneally. IV infusion can be given over as little as 15 minutes, but more often for 30 minutes, 60 minutes, 90 minutes or even 2 or 3 hours. The PSMA binding FN3 domains of the invention may also be injected directly into the site of disease (e.g., the tumor itself). The dose given to a patient having a cancer is sufficient to alleviate or at least partially arrest the disease being treated (“therapeutically effective amount”) and may be sometimes 0.1 to 10 mg/kg body weight, for example 1, 2, 3, 4, 5, 6, 7, 8, 9 or 10 mg/kg, but may even higher, for example 15, 20, 30, 40, 50, 60, 70, 80, 90 or 100 mg/kg. A fixed unit dose may also be given, for example, 50, 100, 200, 500 or 1000 mg, or the dose may be based on the patient's surface area, e.g., 400, 300, 250, 200, or 100 mg/m². Usually between 1 and 8 doses, (e.g., 1, 2, 3, 4, 5, 6, 7 or 8) may be administered to treat cancer, but 10, 12, 20 or more doses may be given. Administration of the FN3 domains of the invention may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose.

For example, a pharmaceutical composition of the FN3 domains of the invention for intravenous infusion may be made up to contain about 200 ml of sterile Ringer's solution, and about 8 mg to about 2400 mg, about 400 mg to about 1600 mg, or about 400 mg to about 800 mg of the PSMA binding FN3 domains for administration to a 80 kg patient. Methods for preparing parenterally administrable compositions are well known and are described in more detail in, for example, “Remington's Pharmaceutical Science”, 15th ed., Mack Publishing Company, Easton, Pa.

The FN3 domains of the invention may be lyophilized for storage and reconstituted in a suitable carrier prior to use. This technique has been shown to be effective with conventional protein preparations and art-known lyophilization and reconstitution techniques can be employed.

The FN3 domains of the invention may be administered to a subject in a single dose or the administration may be repeated, e.g. after one day, two days, three days, five days, six days, one week, two weeks, three weeks, one month, five weeks, six weeks, seven weeks, two months or three months. The repeated administration can be at the same dose or at a different dose. The administration can be repeated once, twice, three times, four times, five times, six times, seven times, eight times, nine times, ten times, or more.

The FN3 domains of the invention may be administered in combination with a second therapeutic agent simultaneously, sequentially or separately.

The FN3 domain of the invention, optionally in combination with the second therapeutic agent may be administered together with any form of radiation therapy including external beam radiation, intensity modulated radiation therapy (IMRT) and any form of radiosurgery including Gamma Knife, Cyberknife, Linac, and interstitial radiation (e.g. implanted radioactive seeds, GliaSite balloon), and/or with surgery.

With particular regard to treatment of solid tumors, protocols for assessing endpoints and anti-tumor activity are well-known in the art. While each protocol may define tumor response assessments differently, the RECIST (Response evaluation Criteria in solid tumors) criteria is currently considered to be the recommended guidelines for assessment of tumor response by the National Cancer Institute. According to the RECIST criteria tumor response means a reduction or elimination of all measurable lesions or metastases. Disease is generally considered measurable if it comprises lesions that can be accurately measured in at least one dimension as ≥20 mm with conventional techniques or 10 mm with spiral CT scan with clearly defined margins by medical photograph or X-ray, computerized axial tomography (CT), magnetic resonance imaging (MRI), or clinical examination (if lesions are superficial). Non-measurable disease means the disease comprises of lesions <20 mm with conventional techniques or <10 mm with spiral CT scan, and truly non-measurable lesions (too small to accurately measure). Non-measurable disease includes pleural effusions, ascites, and disease documented by indirect evidence.

The criteria for objective status are required for protocols to assess solid tumor response. Representative criteria include the following: (1) Complete Response (CR), defined as complete disappearance of all measurable disease; no new lesions; no disease related symptoms; no evidence of non-measurable disease; (2) Partial Response (PR) defined as 30% decrease in the sum of the longest diameter of target lesions (3) Progressive Disease (PD), defined as 20% increase in the sum of the longest diameter of target lesions or appearance of any new lesion; (4) Stable or No Response, defined as not qualifying for CR, PR, or Progressive Disease. Additional endpoints that are accepted within the oncology art include overall survival (OS), disease-free survival (DFS), objective response rate (ORR), time to progression (TTP), and progression-free survival (PFS).

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition

Pharmaceutical compositions provided herein may also comprise the cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In some embodiments, the macrophages provided for herein are administered in an “an immunologically effective amount”, “an anti-immune response effective amount”, “an immune response-inhibiting effective amount”, or “therapeutic amount” as indicated. The amount of the compositions to be administered can be determined by a physician with consideration of individual differences in age, weight, immune response, and condition of the patient (subject). It some embodiments, a pharmaceutical composition comprising the cells described herein may be administered at a dosage of 10⁴ to 10⁹ cells/kg body weight, 10⁵ to 10⁶ cells/kg body weight, including all integer values within those ranges. The cell compositions described herein may also be administered multiple times at these dosages. The cells can be administered by using infusion techniques that are commonly known in immunotherapy (see, e.g., Rosenberg et al., New Eng. J. of Med. 319:1676, 1988, which is hereby incorporated by reference).

In some embodiments, it may be desired to administer the macrophages to a subject and then subsequently redraw blood (or have an apheresis performed), activate the macrophages therefrom, and reinfuse the patient with these activated cells. This process can be carried out multiple times every few weeks. In some embodiments, the cells can be activated from blood draws of from 10 ml to 400 ml. In some embodiments, the cells are activated from blood draws of 20 ml, 30 ml, 40 ml, 50 ml, 60 ml, 70 ml, 80 ml, 90 ml, or 100 ml.

While having described embodiments in general terms, the embodiments provided herein will be further disclosed in the following examples that should not be construed as limiting the scope of the claims.

### EXAMPLES

### Reagents and Constructs:

The extracellular domains of cynomolgus (cyno monkey protein database ref #EHH56646.1, SEQ ID NO: 32) and chimpanzee (Uniprot, Ref #H2Q3K5, SEQ ID NO: 33) PSMA were cloned into the pUnder expression vector along with a 6His and Avi tag. Proteins were transiently expressed in 293HEK-expi cells. Supernatants were harvested and clarified by centrifugation. The proteins were purified using a two-step purificationprocess: 1) IMAC purification with a HisTrap HP column and 2) size exclusion purification (Superdex 200) where the elution buffer is DPBS containing Mg²⁺, Ca²⁺, and 0.5 mM ZnCl2 to stabilize PSMA dimerization. Fractions containing the protein of interest were pooled and protein concentration was determined by A280.

The gene encoding S. aureus sortase A was produced by DNA2.0 and subcloned into pJexpress401 vector (DNA2.0) for expression under the T5 promoter. The sortase construct for soluble expression is lacking the N-terminal domain of the natural protein consisting of 25 amino acids since this domain is membrane associated. The sortase was expressed as N-terminal His6-tag (HHHHHH, SEQ ID NO: 34) followed by a TEV protease site for tag removal (ENLYFQS, SEQ ID NO: 54), resulting in the sortase having the amino acid sequence of SEQ ID NO: 52. The sortase protein used also includes 5 mutations sequence that are reported to increase the catalytic efficiency of the enzyme when compared to the wild type proteins (SEQ ID NO: 53). The plasmid was transformed into E. coli BL21 Gold cells (Agilent) for expression. A single colony was picked and grown in Luria Broth (Teknova) supplemented with kanamycin and incubated 18 h at 37° C. 250 RPM. 250 mL of Terrific Broth (Teknova), supplemented with kanamycin, was inoculated from these subcultures and grown at 37° C. for ˜4 h while shaking. Protein expression was induced with 1 mM IPTG, and the protein was expressed for 18 h at 30° C. Cells were harvested by centrifugation at 6000 g and stored at −20 C until purification. The frozen cell pellet was thawed for 30 min at room temperature and suspended in BugBusterHT protein extraction reagent (EMD Millipore) supplemented with 1 uL per 30 mL of recombinant lysozyme (EMD Millipore) at 5 ml per gram of cell paste and incubated for 30 minutes on a shaker at room temperature. The lysate was clarified by centrifugation at 74 600 g for 30 min.

The supernatant was applied onto a gravity column packed with 3 mL of Qiagen Superflow Ni-NTA resin pre-equilibrated with buffer A (50 mM sodium phosphate buffer, pH 7.0 containing 0.5 M NaCl and 10 mM imidazole). After loading, the column was washed with 100 mL of Buffer A. The protein was eluted with Buffer A supplemented with 250 mM imidazole and loaded on a preparative gel-filtration column, TSK Gel G3000SW 21.5×600 mm (Tosoh) equilibrated in PBS (Gibco). The gel-filtration chromatography was performed at room temperature in PBS at flow rate 10 ml/min using an AKTA-AVANT chromatography system. Purified sortase was then digested with TEV protease to remove the His6 tag. 28 mgs of sortase was incubated in 10 mLs with 3000 units of AcTEV protease (Invitrogen) in the supplied buffer supplemented with 1 mM DTT for 2 hours at 30 C. The tagless sortase was purified with Ni-NTA resin. The reaction was exchanged into TBS buffer (50 mM Tris pH 7.5, 150 mM NaCl) using PD-10 columns (GE Healthcare) and applied onto a gravity column packed with 0.5 mL of Qiagen Superflow Ni-NTA resin pre-equilibrated with buffer A. The flowthrough was collected and the resin was washed with 3 mL of buffer A which was added to the flowthrough. This flowthrough was concentrated to ˜0.5 mL in an Amicon 15 concentrator with 10 kDa cutoff (EMD Millipore). Additional TBS buffer was added and the sample was concentrated again (repeated twice) to exchange the buffer to TBS. ⅓rd volume of 40% glycerol was added (final concentration of 10% glycerol), and the sortase was stored at −20 C for short term use or −80 C for long term.

### Example 1. Construction of Tencon Libraries with Randomized Loops

Tencon (SEQ ID NO: 1) is an immunoglobulin-like scaffold, fibronectin type III (FN3) domain, designed from a consensus sequence of fifteen FN3 domains from human tenascin-C (Jacobs et al., Protein Engineering, Design, and Selection, 25:107-117, 2012; U.S. Pat. No. 8,278,419). The crystal structure of Tencon shows six surface-exposed loops that connect seven beta-strands. These loops, or selected residues within each loop, can be randomized in order to construct libraries of fibronectin type III (FN3) domains that can be used to select novel molecules that bind to specific targets.

Tencon: (SEQ ID NO 1):
LPAPKNLVVSEVTEDSLRLSWTAPDAAFDSFLIQYQESEKVGEAINL
TVPGSERSYDLTGLKPGTEYTVSIYGVKGGHRSNPLSAEFTT

Various libraries were generated using the tencon scaffold and various design strategies. In general, libraries TCL1 and TCL2 produced good binders. Generation of TCL1 and TCL2 libraries are described in detail in Int. Pat. Publ. No. WO2014081944A2.

### Construction of TCL1 Library

A library designed to randomize only the FG loop of Tencon (SEQ ID NO: 1), TCL1, was constructed for use with the cis-display system. In this system, a single-strand DNA incorporating sequences for a Tac promoter, Tencon library coding sequence, RepA coding sequence, cis-element, and ori element is produced. Upon expression in an in vitro transcription/translation system, a complex is produced of the Tencon-RepA fusion protein bound in cis to the DNA from which it is encoded. Complexes that bind to a target molecule are then isolated and amplified by polymerase chain reaction (PCR), as described below.

Construction of the TCL1 library for use with cis-display was achieved by successive rounds of PCR to produce the final linear, double-stranded DNA molecules in two halves; the 5′ fragment contains the promoter and Tencon sequences, while the 3′ fragment contains the repA gene and the cis- and ori elements. These two halves are combined by restriction digest in order to produce the entire construct. The TCL1 library was designed to incorporate random amino acids only in the FG loop of Tencon, KGGHRSN (SEQ ID NO: 55). NNS codons were used in the construction of this library, resulting in the possible incorporation of all 20 amino acids and one stop codon into the FG loop. The TCL1 library contains six separate sub-libraries, each having a different randomized FG loop length, from 7 to 12 residues, in order to further increase diversity.

TCL1 library
(SEQ ID NO: 2)
LPAPKNLVVSEVTEDSLRLSWTAPDAAFDSFLIQYQESEKVGEAINLTVP
GSERSYDLTGLKPGTEYTVSIYGVX₇₋₁₂PLSAEFTT;

wherein
X₁, X₂, X₃, X₄, X₅, X₆, X₇ is any amino acid; and
X₈, X₉, X₁₀, X₁₁ and X₁₂ are any amino acid or deleted

### Construction of TCL2 Library

TCL2 library was constructed in which both the BC and the FG loops of Tencon were randomized and the distribution of amino acids at each position was strictly controlled. Table 3 shows the amino acid distribution at desired loop positions in the TCL2 library. The designed amino acid distribution had two aims. First, the library was biased toward residues that were predicted to be structurally important for Tencon folding and stability based on analysis of the Tencon crystal structure and/or from homology modeling. For example, position 29 was fixed to be only a subset of hydrophobic amino acids, as this residue was buried in the hydrophobic core of the Tencon fold. A second layer of design included biasing the amino acid distribution toward that of residues preferentially found in the heavy chain HCDR3 of antibodies, to efficiently produce high-affinity binders. Towards this goal, the “designed distribution” in Table 2 refers to the distribution as follows: 6% alanine, 6% arginine, 3.9% asparagine, 7.5% aspartic acid, 2.5% glutamic acid, 1.5% glutamine, 15% glycine, 2.3% histidine, 2.5% isoleucine, 5% leucine, 1.5% lysine, 2.5% phenylalanine, 4% proline, 10% serine, 4.5% threonine, 4% tryptophan, 17.3% tyrosine, and 4% valine. This distribution is devoid of methionine, cysteine, and STOP codons.

TCL2 library
(SEQ ID NO: 3)
LPAPKNLVVSEVTEDSLRLSWX₁X₂X₃X₄X₅X₆X₇X₈SFLIQYQESEKVG
EAINLTVPGSERSYDLTGLKPGTEYTVSIYGVX₉X₁₀X₁₁X₁₂X₁₃SX₁₄
X₁₅LSAEFTT;

wherein

### X₁ is Ala, Arg, Asn, Asp, Glu, Gln, Gly, His, Ile, Leu, Lys, Phe, Pro, Ser, Thr, Trp, Tyr or Val;

### X₂ is Ala, Arg, Asn, Asp, Glu, Gln, Gly, His, Ile, Leu, Lys, Phe, Pro, Ser, Thr, Trp, Tyr or Val;

### X₃ Ala, Arg, Asn, Asp, Glu, Gln, Gly, His, Ile, Leu, Lys, Phe, Pro, Ser, Thr, Trp, Tyr or Val;

### X₄ is Ala, Arg, Asn, Asp, Glu, Gln, Gly, His, Ile, Leu, Lys, Phe, Pro, Ser, Thr, Trp, Tyr or Val;

### X₅ is Ala, Arg, Asn, Asp, Glu, Gln, Gly, His, Ile, Leu, Lys, Phe, Pro, Ser, Thr, Trp, Tyr or Val;

### X₆ is Ala, Arg, Asn, Asp, Glu, Gln, Gly, His, Ile, Leu, Lys, Phe, Pro, Ser, Thr, Trp, Tyr or Val;

### X₇ is Phe, Ile, Leu, Val or Tyr;

### X₈ is Asp, Glu or Thr;

### X₉ is Ala, Arg, Asn, Asp, Glu, Gln, Gly, His, Ile, Leu, Lys, Phe, Pro, Ser, Thr, Trp, Tyr or Val;

### X₁₀ is Ala, Arg, Asn, Asp, Glu, Gln, Gly, His, Ile, Leu, Lys, Phe, Pro, Ser, Thr, Trp, Tyr or Val;

### X₁₁ is Ala, Arg, Asn, Asp, Glu, Gln, Gly, His, Ile, Leu, Lys, Phe, Pro, Ser, Thr, Trp, Tyr or Val;

### X₁₂ is Ala, Arg, Asn, Asp, Glu, Gln, Gly, His, Ile, Leu, Lys, Phe, Pro, Ser, Thr, Trp, Tyr or Val;

### X₁₃ is Ala, Arg, Asn, Asp, Glu, Gln, Gly, His, Ile, Leu, Lys, Phe, Pro, Ser, Thr, Trp, Tyr or Val;

### X₁₄ is Ala, Arg, Asn, Asp, Glu, Gln, Gly, His, Ile, Leu, Lys, Phe, Pro, Ser, Thr, Trp, Tyr or Val; and

### X₁₅ is Ala, Arg, Asn, Asp, Glu, Gln, Gly, His, Ile, Leu, Lys, Phe, Pro, Ser, Thr, Trp, Tyr or Val.

TABLE 2

| Residue | WT | |
|---|---|---|
| Position* | residues | Distribution in the TCL2 library |
| 22 | T | designed distribution |
| 23 | A | designed distribution |
| 24 | P | 50% P + designed distribution |
| 25 | D | designed distribution |
| 26 | A | 20% A + 20% G + designed distribution |
| 27 | A | designed distribution |
| 28 | F | 20% F, 20% I, 20% L, 20% V, 20% Y |
| 29 | D | 33% D, 33% E, 33% T |
| 75 | K | designed distribution |
| 76 | G | designed distribution |
| 77 | G | designed distribution |
| 78 | H | designed distribution |
| 79 | R | designed distribution |
| 80 | S | 100% S |
| 81 | N | designed distribution |
| 82 | P | 50% P + designed distribution |

*residue numbering is based on Tencon sequence of SEQ ID NO: 1

Subsequently, these libraries were improved by various ways, including building of the libraries on a stabilized Tencon framework (U.S. Pat. No. 8,569,227) that incorporates substitutions E11R/L17A/N46V/E86I (Tencon27; SEQ ID NO: 4) when compared to the wild type tencon as well as altering of the positions randomized in the BC and FG loops. Tencon27 is described in Int. Pat. Appl. No. WO2013049275. From this, new libraries designed to randomize only the FG loop of Tencon (library TCL9), or a combination of the BC and FG loops (library TCL7) were generated. These libraries were constructed for use with the cis-display system. The details of this design are shown below:

Stabilized Tencon (Tencon27)
(SEQ ID NO: 4)
LPAPKNLVVSRVTEDSARLSWTAPDAAFDSFLIQYQESEKVGEAIVLTVP
GSERSYDLTGLKPGTEYTVSIYGVKGGHRSNPLSAIFTT
TCL7 (randomized EG and BC loops)
(SEQ ID NO: 5)
LPAPKNLVVSRVTEDSARLSWX₁X₂X₃X₄X₅X₆X₇X₈X₉FDSFLIQYQES
EKVGEAIVLTVPGSERSYDLTGLKPGTEYTVSIYGVX₁₀X₁₁X₁₂X₁₃
X₁₄X₁₅X₁₆X₁₇X₁₈X₁₉SNPLSAIFTT;

wherein
X₁, X₂, X₃, X₄, X₅, X₆, X₁₀, X₁₁, X₁₂, X₁₃, X₁₄, X₁₅ and X₁₆ is A, D, E, F, G, H, I, K, L, N, P, Q, R, S, T, V, W or Y; and
X₇, X₈, X₉, X₁₇, X₁₈ and X₁₉, is A, D, E, F, G, H, I, K, L, N, P, Q, R, S, T, V, W, Y or deleted.

TCL9 (randomized FG loop)
(SEQ ID NO: 6)
LPAPKNLVVSRVTEDSARLSWTAPDAAFDSFLIQYQESEKVGEAIVLTV
PGSERSYDLTGLKPGTEYTVSIYGV X₁X₂X₃X₄X₅X₆X₇X₈X₉ X₁₀X₁₁X₁₂
SNPLSAIFTT;

X₁, X₂, X₃, X₄, X₅, X₆ and X₇, is A, D, E, F, G, H, I, K, L, N, P, Q, R, S, T, V, W or Y; and
X₈, X₉, X₁₀, X₁₁ and X₁₂ is A, D, E, F, G, H, I, K, L, N, P, Q, R, S, T, V, W, Y or deleted.

For library construction, DNA fragments encoding randomized BC loops (lengths 6-9 positions) or FG loops (lengths 7-12 positions) were synthesized using Slonomics technology (Sloning Biotechnology GmbH) so as to control the amino acid distribution of the library and to eliminate stop codons. Two different sets of DNA molecules randomizing either the BC loop or the FG loops were synthesized independently and later combined using PCR to produce the full library product.

### Construction of FG Loop Libraries (TCL9)

A set of synthetic DNA molecules consisting of a 5′ Tac promoter followed by the complete gene sequence of Tencon with the exception of randomized codons in the FG loop was produced (SEQ ID NOs: 26-31). For FG loop randomization, all amino acids except cysteine and methionine were encoded at equal percentages. The lengths of the diversified portion are such that they encode for 7, 8, 9, 10, 11, or 12 amino acids in the FG loop. Sub-libraries of each length variation were synthesized individually at a scale of 2 ug and then amplified by PCR using oligos Sloning-FOR (SEQ ID NO: 9) and Sloning-Rev (SEQ ID NO: 10).

The 3′ fragment of the library is a constant DNA sequence containing elements for display, including a PspOMI restriction site, the coding region of the repA gene, and the cis- and ori elements. PCR reactions were performed to amplify this fragment using a plasmid (pCR4Blunt) (Invitrogen) as a template with M13 Forward and M13 Reverse primers. The resulting PCR products were digested by PspOMI overnight and gel-purified. To ligate the 5′ portion of library DNA to the 3′ DNA containing repA gene, 2 pmol (˜540 ng to 560 ng) of 5′ DNA was ligated to an equal molar (˜1.25 μg) of 3′ repA DNA in the presence of NotI and PspOMI enzyme and T4 ligase at 37° C. overnight. The ligated library product was amplified by using 12 cycles of PCR with oligos POP2250 (SEQ ID NO: 11) and DigLigRev (SEQ ID NO: 12). For each sub-library, the resulting DNA from 12 PCR reactions were combined and purified by Qiagen spin column. The yield for each sub-library of TCL9 ranged from 32-34 μg.

### Construction of FG/BC Loop Libraries (TCL7)

The TCL7 library provides for a library with randomized Tencon BC and FG loops. In this library, BC loops of lengths 6-9 amino acids were mixed combinatorially with randomized FG loops of 7-12 amino acids in length. Synthetic Tencon fragments BC6, BC7, BC8, and BC9 (SEQ ID No. 13-16) were produced to include the Tencon gene encoding for the N-terminal portion of the protein up to and including residue VX such that the BC loop is replaced with either 6, 7, 8, or 9 randomized amino acids. These fragments were synthesized prior to the discovery of L17A, N46V and E83I mutations (CEN5243) but these mutations were introduced in the molecular biology steps described below. In order to combine this fragment with fragments encoding for randomized FG loops, the following steps were taken.

First, a DNA fragment encoding the Tac promoter and the 5′ sequence of Tencon up to the nucleotide endoding for amino acid A17 (130mer-L17A, SEQ ID No. 17) was produced by PCR using oligos POP2222ext (SEQ ID No. 18) and LS1114 (SEQ ID No. 19). This was done to include the L17A mutation in the library (CEN5243). Next, DNA fragments encoding for Tencon residues R18-V75 including randomized BC loops were amplified by PCR using BC6, BC7, BC8, or BC9 as a templates and oligos LS1115 (SEQ ID No. 20) and LS1117 (SEQ ID No. 21). This PCR step introduced a BsaI site at the 3′ end. These DNA fragments were subsequently joined by overlapping PCR using oligos POP2222ext and LS1117 as primers. The resulting PCR product of 240 bp was pooled and purified by Qiagen PCR purification kit. The purified DNA was digested with BsaI-HF and gel purified.

Fragments encoding the FG loop were amplified by PCR using FG7, FG8, FG9, FG10, FG11, and FG12 as templates with oligonucleotides SDG10 (SEQ ID No. 22) and SDG24 (SEQ ID No. 23) to incorporate a BsaI restriction site and N46V and E86I variations (CEN5243).

The digested BC fragments and FG fragments were ligated together in a single step using a 3-way ligation. Four ligation reactions in the 16 possible combinations were set up, with each ligation reaction combining two BC loop lengths with 2 FG loop lengths. Each ligation contained ˜300 ng of total BC fragment and 300 ng of the FG fragment. These 4 ligation pools were then amplified by PCR using oligos POP2222 (SEQ ID No. 24) and SDG28 SEQ ID No. 25). 7.5 μg of each reaction product were then digested with Not1 and cleaned up with a Qiagen PCR purification column. 5.2 μg of this DNA, was ligated to an equal molar amount of RepA DNA fragment (˜14 μg) digested with PspOMI and the product amplified by PCR using oligos POP2222.

### Example 2: Generation of Tencon Libraries Having Alternative Binding Surfaces

The choice of residues to be randomized in a particular library design governs the overall shape of the interaction surface created. X-ray crystallographic analysis of an FN3 domain containing scaffold protein selected to bind maltose binding protein (MBP) from a library in which the BC, DE, and FG loops were randomized was shown to have a largely curved interface that fits into the active site of MBP. In contrast, an ankyrin repeat scaffold protein that was selected to bind to MBP was found to have a much more planar interaction surface and to bind to the outer surface of MBP distant from the active. These results suggest that the shape of the binding surface of a scaffold molecule (curved vs. flat) may dictate what target proteins or specific epitopes on those target proteins are able to be bound effectively by the scaffold. Published efforts around engineering protein scaffolds containing FN3 domains for protein binding has relied on engineering adjacent loops for target binding, thus producing curved binding surfaces. This approach may limit the number of targets and epitopes accessible by such scaffolds.

Tencon and other FN3 domains contain two sets of CDR-like loops lying on the opposite faces of the molecule, the first set formed by the BC, DE, and FG loops, and the second set formed by the AB, CD, and EF loops. The two sets of loops are separated by the beta-strands that form the center of the FN3 structure. If the image of the Tencon is rotated by 90 degrees, an alternative surface can be visualized. This slightly concave surface is formed by the CD and FG loops and two antiparallel beta-strands, the C and the F beta-strands, and is herein called the C-CD-F-FG surface. The C-CD-F-FG surface can be used as a template to design libraries of protein scaffold interaction surfaces by randomizing a subset of residues that form the surface. Beta-strands have a repeating structure with the side chain of every other residue exposed to the surface of the protein. Thus, a library can be made by randomizing some or all surface exposed residues in the beta strands. By choosing the appropriate residues in the beta-strands, the inherent stability of the Tencon scaffold should be minimally compromised while providing a unique scaffold surface for interaction with other proteins.

Library TCL14 (SEQ ID NO: 7), was designed into Tencon27 scaffold (SEQ ID NO: 4).

A full description of the methods used to construct this library is described in US. Pat. Publ. No. US2013/0226834.

TCL14 library (SEQ ID NO: 7):
LPAPKNLVVSRVTEDSARLSWTAPDAAFDSFX₁IX₂YX₃EX₄X₅X₆X₇GE
AIVLTVPGSERSYDLTGLKPGTEYX₈VX₉IX₁₀GVKGGX₁₁X₁₂SX₁₃PL
SAIFTT;

wherein
X₁, X₂, X₃, X₄, X₅, X₆, X₇, X₈, X₉, X₁₀, X₁₁, X₁₂ and X₁₃ are A, D, E, F, G, H, I, K, L, N, P, Q, R, S, T, V, W, Y, C or M.

The two beta strands forming the C-CD-F-FG surface in Tencon27 have a total of 9 surface exposed residues that could be randomized; C-strand: S30, L32, Q34, Q36; F-strand: E66, T68, S70, Y72, and V74, while the CD loop has 6 potential residues: S38, E39, K40, V41, G42, and E43 and the FG loop has 7 potential residues: K75, G76, G77, H78, R79, S80, and N81. Select residues were chosen for inclusion in the TCL14 design due to the larger theoretical size of the library if all 22 residues were randomized.

Thirteen positions in Tencon were chosen for randomizing L32, Q34 and Q36 in C-strand, S38, E39, K40 and V41 in CD-loop, T68, S70 and Y72 in F-strand, H78, R79, and N81 in FG-loop. In the C and F strands S30 and E66 were not randomized as they lie just beyond the CD and FG loops and do not appear to be as apparently a part of the C-CD-F-FG surface. For the CD loop, G42 and E43 were not randomized as glycine, providing flexibility, can be valuable in loop regions, and E43 lies at the junction of the surface. The FG loop had K75, G76, G77, and S80 excluded. The glycines were excluded for the reasons above while careful inspection of the crystal structures revealed S80 making key contacts with the core to help form the stable FG loop. K75 faces away from the surface of the C-CD-F-FG surface and was a less appealing candidate for randomization. Although the above mentioned residues were not randomized in the original TCL14 design, they could be included in subsequent library designs to provide additional diversity for de novo selection or for example for an affinity maturation library on a select TCL14 target specific hit.

Subsequent to the production of TCL14, 3 additional Tencon libraries of similar design were produced. These two libraries, TCL19, TCL21 and TCL23, are randomized at the same positions as TCL14 (see above) however the distribution of amino acids occurring at these positions is altered (Table 3). TCL19 and TCL21 were designed to include an equal distribution of 18 natural amino acids at every position (5.55% of each), excluding only cysteine and methionine. TCL23 was designed such that each randomized position approximates the amino acid distribution found in the HCDR3 loops of functional antibodies as described in Table 3. As with the TCL21 library, cysteine and methionine were excluded.

A third additional library was built to expand potential target binding surface of the other libraries library. In this library, TCL24, 4 additional Tencon positions were randomized as compared to libraries TCL14, TCL19, TCL21, and TCL23. These positions include N46 and T48 from the D strand and S84 and 186 from the G strand. Positions 46, 48, 84, and 86 were chosen in particular as the side chains of these residues are surface exposed from beta-strands D and G and lie structurally adjacent to the randomized portions of the C and F strand, thus increasing the surface area accessible for binding to target proteins. The amino acid distribution used at each position for TCL24 is identical to that described for TCL19 and TCL21 in Table 3.

TCL24 Library
(SEQ ID NO: 8)
LPAPKNLVVSRVTEDSARLSWTAPDAAFDSFX₁IX₂YX₃EX₄X₅X₆X₇GE
AIX₈LX₉VPGSERSYDLTGLKPGTEYX₁₀VX₁₁IX₁₂GVKGGX₁₃X₁₄S
X₁₅PLX₁₆AX₁₇FTT;

wherein
X₁, X₂, X₃, X₄, X₅, X₆, X₁₀, X₁₁, X₁₂, X₁₃, X₁₄, X₁₅, X₁₆ and X₁₇ are A, D, E, F, G, H, I, K, L, N, P, Q, R, S, T, V, Y or W.

TABLE 3
Amino acid frequency (%) at each randomized
position for TCL21, TCL23, and TCL24.

| Amino Acid | TCL19 | TCL21 | TCL23 | TCL24 |
|---|---|---|---|---|
| Ala | 5.6 | 5.6 | 6.0 | 5.6 |
| Arg | 5.6 | 5.6 | 6.0 | 5.6 |
| Asn | 5.6 | 5.6 | 3.9 | 5.6 |
| Asp | 5.6 | 5.6 | 7.5 | 5.6 |
| Cys | 0.0 | 0.0 | 0.0 | 0.0 |
| Gln | 5.6 | 5.6 | 1.5 | 5.6 |
| Glu | 5.6 | 5.6 | 2.5 | 5.6 |
| Gly | 5.6 | 5.6 | 15.0 | 5.6 |
| His | 5.6 | 5.6 | 2.3 | 5.6 |
| Ile | 5.6 | 5.6 | 2.5 | 5.6 |
| Leu | 5.6 | 5.6 | 5.0 | 5.6 |
| Lys | 5.6 | 5.6 | 1.5 | 5.6 |
| Met | 0.0 | 0.0 | 0.0 | 0.0 |
| Phe | 5.6 | 5.6 | 2.5 | 5.6 |
| Pro | 5.6 | 5.6 | 4.0 | 5.6 |
| Ser | 5.6 | 5.6 | 10.0 | 5.6 |
| Thr | 5.6 | 5.6 | 4.5 | 5.6 |
| Trp | 5.6 | 5.6 | 4.0 | 5.6 |
| Tyr | 5.6 | 5.6 | 17.3 | 5.6 |
| Val | 5.6 | 5.6 | 4.0 | 5.6 |

### Generation of TCL21, TCL23, and TCL24 Libraries

The TCL21 library was generated using Colibra library technology (Isogenica) in order to control amino acid distributions. TCL19, TCL23, and TCL24 gene fragments were generated using Slonomics technology (Morphosys) to control amino acid distributions. PCR was used to amplify each library following initial synthesis followed by ligation to the gene for RepA in order to be used in selections using the CIS-display system as described above for the loop libraries.

### Example 3: Selection of Fibronectin Type III (FN3) Domains that Bind PSMA

### Plate-Based Selections

CIS-display was used to select PSMA binding Centyrins from the TCL7, TCL9, TCL19, and TCL21 libraries. For in vitro transcription and translation (ITT), 3 μg of library DNA were incubated at 30° C. with 0.1 mM complete amino acids, 1× S30 premix components, and 15 μL of S30 extract (Promega) in a total volume of 50 μL. After 1 hour, 375 μL of blocking solution (lx TBS pH 7.4, 0.01% I-block (Life Technologies, #T2015), 100 ug/ml herring sperm DNA) was added and reactions were incubated on ice for 15 minutes. ITT reactions were incubated with recombinant proteins, chimpanzee (pan 229) or cynomolgus monkey PSMA (pan 230), or cynomolgus monkey PSMA-Fc fusion (pan 231), which were immobilized on anti-human PSMA antibody (Lifespan Bioscience, catalog #LC-C150527) coated 96-well Maxisorb plates. Unbound library members were removed by successive washes with TBST and TBS. After washing, DNA was eluted from the target protein by heating to 85° C. for 10 minutes and amplified by PCR for further rounds of panning High affinity binders were isolated by successively lowering the concentration of target PSMA during each round from 400 nM to 100 nM and increasing the washing stringency.

Following panning, selected FN3 domains were amplified by PCR, subcloned into a pET vector modified to include a ligase independent cloning site, and transformed into BL21-GOLD (DE3) (Stratagene) cells for soluble expression in E. coli using standard molecular biology techniques. A gene sequence encoding a C-terminal poly-histidine tag was added to each FN3 domain to enable purification and detection. Cultures were grown to an optical density of 0.6-0.8 in TB medium supplemented with 100 μg/mL carbenicillin in 1-mL 96-well blocks at 37° C. before the addition of IPTG to 1 mM, at which point the temperature was reduced to 30° C. Cells were harvested approximately 16 hours later by centrifugation and frozen at −20° C. Cell lysis was achieved by incubating each pellet in 0.6 mL of BugBuster® HT lysis buffer (Novagen EMD Biosciences) with shaking at room temperature for 45 minutes.

### Bead-Based Selections

Centyrins were also selected using a bead-based capture setup. ITT reactions were prepared as described above and then incubated with biotinylated recombinant proteins, chimpanzee or cynomolgus monkey PSMA. The biotinylated recombinant proteins and the bound library members were captured on neutravidin or streptavidin coated magnetic beads. Unbound library members were removed by successive washes with TBST and TBS. After washing, DNA was eluted from the target protein by heating to 85° C. for 10 minutes and amplified by PCR for further rounds of panning High affinity binders were isolated by successively lowering the concentration of target PSMA during each round from 400 nM to 100 nM and increasing the washing stringency.

### Off-Rate Selections

Outputs from the fifth round of bead-based selection were subjected to four rounds of off-rate selection. After the ITT reactions were incubated with biotinylated recombinant chimpanzee or cynomolgus monkey proteins, the proteins and the bound library members were captured on neutravidin or streptavidin coated magnetic beads, and washed in TBST extensively, the bound complexes were washed in 5 μM cold recombinant PSMA proteins for 1 hour. Then the ITT bound to beads were washed extensively in TBST and TBS before being eluted. The biotinylated target antigen concentration was stepped down from 25 nM in rounds 6 and 7 to 2.5 nM in rounds 8 and 9. Selection outputs from rounds 7 and 9 were subcloned into the modified pET15 vector for expression and screening.

### Affinity Maturation Library Selection

An affinity maturation library (TCL25) based on the sequence of clone P229CR9P819-H11 (SEQ ID NO: 40) was generated using Slonomics technology at Morphosys (Munich, Germany) in which positions 23-30 from the BC loop and positions 78-83 from the FG loop were randomized Maintenance of target binding in the library was achieved by doping nucleotides encoding the parent amino acid (from P229CR9P819-H11) at a target frequency of 65% at each randomized position. The remaining 35% of nucleotides were designed to contain a mixture of codons encoding for an equal probability of all other 20 natural amino acids, with the exception of cysteine and methionine which were not included. Table 4 shows the design of the TCL25 maturation library. In the table, numbers in parenthesis represent the percentage of molecules in the library designed to contain the corresponding amino acid at each position. This doping scheme (65% parent at 14 positions) generates a theoretical distribution of molecules containing mostly 3, 4, 5, 6, or 7 changes as compared to the parent molecule.

TABLE 4

| | Parent | |
|---|---|---|
| | Amino | |
| Position | Acid | Amino Acid Distribution (%) |
| 23 | Asp | (2.05), arg (2.05), asn (2.05), asp (65), gln (2.05), |
| | | glu (2.05), gly (2.05), his (2.05), ile (2.05), leu |
| | | (2.05), lys (2.05), phe (2.05), pro (2.05), ser (2.05), |
| | | thr (2.05), tyr(2.05), trp (2.05), val (2.05) |
| 24 | Ile | ala (2.05), arg (2.05), asn (2.05), asp (2.05), gln |
| | | (2.05), glu (2.05), gly (2.05), his (2.05), ile (65), |
| | | leu (2.05), lys (2.05), phe (2.05), pro (2.05), ser |
| | | (2.05), thr (2.05), tyr(2.05), trp (2.05), val (2.05) |
| 25 | Asp | ala (2.05), arg (2.05), asn (2.05), asp (65), gln |
| | | (2.05), glu (2.05), gly (2.05), his (2.05), ile (2.05), |
| | | leu (2.05), lys (2.05), phe (2.05), pro (2.05), ser |
| | | (2.05), thr (2.05), tyr(2.05), trp (2.05), val (2.05) |
| 26 | Glu | ala (2.05), arg (2.05), asn (2.05), asp (2.05), gln |
| | | (2.05), glu (65), gly (2.05), his (2.05), ile (2.05), |
| | | leu (2.05), lys (2.05), phe (2.05), pro (2.05), ser |
| | | (2.05), thr (2.05), tyr(2.05), trp (2.05), val (2.05) |
| 27 | Gln | ala (2.05), arg (2.05), asn (2.05), asp (2.05), gln |
| | | (65), glu (2.05), gly (2.05), his (2.05), ile (2.05), |
| | | leu (2.05), lys (2.05), phe (2.05), pro (2.05), ser |
| | | (2.05), thr (2.05), tyr(2.05), trp (2.05), val (2.05) |
| 28 | Arg | ala (2.05), arg (65), asn (2.05), asp (2.05), gln |
| | | (2.05), glu (2.05), gly (2.05), his (2.05), ile (2.05), |
| | | leu (2.05), lys (2.05), phe (2.05), pro (2.05), ser |
| | | (2.05), thr (2.05), tyr(2.05), trp (2.05), val (2.05) |
| 29 | Asp | ala (2.05), arg (2.05), asn (2.05), asp (65), gln |
| | | (2.05), glu (2.05), gly (2.05), his (2.05), ile (2.05), |
| | | leu (2.05), lys (2.05), phe (2.05), pro (2.05), ser |
| | | (2.05), thr (2.05), tyr(2.05), trp (2.05), val (2.05) |
| 30 | Trp | ala (2.05), arg (2.05), asn (2.05), asp (2.05), gln |
| | | (2.05), glu (2.05), gly (2.05), his (2.05), ile (2.05), |
| | | leu (2.05), lys (2.05), phe (2.05), pro (2.05), ser |
| | | (2.05), thr (2.05), tyr(2.05), trp (65), val (2.05) |
| 78 | Tyr | ala (2.05), arg (2.05), asn (2.05), asp (2.05), gln |
| | | (2.05), glu (2.05), gly (2.05), his (2.05), ile (2.05), |
| | | leu (2.05), lys (2.05), phe (2.05), pro (2.05), ser |
| | | (2.05), thr (2.05), tyr(65), trp (2.05), val (2.05) |
| 79 | His | ala (2.05), arg (2.05), asn (2.05), asp (2.05), gln |
| | | (2.05), glu (2.05), gly (2.05), his (65), ile (2.05), |
| | | leu (2.05), lys (2.05), phe (2.05), pro (2.05), ser |
| | | (2.05), thr (2.05), tyr(2.05), trp (2.05), val (2.05) |
| 80 | Val | ala (2.05), arg (2.05), asn (2.05), asp (2.05), gln |
| | | (2.05), glu (2.05), gly (2.05), his (2.05), ile (2.05), |
| | | leu (2.05), lys (2.05), phe (2.05), pro (2.05), ser |
| | | (2.05), thr (2.05), tyr(2.05), trp (2.05), val (65) |
| 81 | Tyr | ala (2.05), arg (2.05), asn (2.05), asp (2.05), gln |
| | | (2.05), glu (2.05), gly (2.05), his (2.05), ile (2.05), |
| | | leu (2.05), lys (2.05), phe (2.05), pro (2.05), ser |
| | | (2.05), thr (2.05), tyr(65), trp (2.05), val (2.05) |
| 82 | Arg | ala (2.05), arg (65), asn (2.05), asp (2.05), gln |
| | | (2.05), glu (2.05), gly (2.05), his (2.05), ile (2.05), |
| | | leu (2.05), lys (2.05), phe (2.05), pro (2.05), ser |
| | | (2.05), thr (2.05), tyr(2.05), trp (2.05), val (2.05) |
| 83 | Ser | ala (2.05), arg (2.05), asn (2.05), asp (2.05), gln |
| | | (2.05), glu (2.05), gly (2.05), his (2.05), ile (2.05), |
| | | leu (2.05), lys (2.05), phe (2.05), pro (2.05), ser |
| | | (65), thr (2.05), tyr(2.05), trp (2.05), val (2.05) |

CIS-display was used to select PSMA binding Centyrins from TCL25 library. The ITT reactions were incubated with biotinylated recombinant proteins, chimpanzee or cyno monkey PSMA. The biotinylated recombinant proteins and the bound library members were captured on neutravidin or streptavidin coated magnetic beads. Unbound library members were removed by successive washes with TBST and TBS. After washing, DNA was eluted from the target protein by heating to 85° C. for 10 minutes and amplified by PCR for further rounds of panning Centyrin binders were isolated by successively lowering the concentration of target PSMA during each round from 400 nM to 100 nM and increasing the washing stringency.

Outputs from the second round selection were subjected to four rounds of off-rate selection. After the ITT reactions were incubated biotinylated recombinant PSMA proteins, the proteins and the bound library members were captured on neutravidin or streptavidin coated magnetic beads, and washed in TBST extensively, the bound complexes were washed in 5 μM cold recombinant PSMA proteins for 1 hour. Then the ITT bound to beads were washed extensively in TBST and TBS before being eluted. The biotinylated target antigen concentration was stepped down from 25 nM in rounds 3 and 4 to 2.5 nM in rounds 5 and 6. Selection outputs from rounds 7 and 9 were subcloned into the modified pET15 vector for expression and screening.

Biochemical Screening for Centyrins that Bind PSMA

Neutravidin-coated plates were blocked for 1 h in Starting Block T20 (Pierce) and then coated with biotinylated PSMA (using same antigen as in panning) or negative control for 1 h. Plates were rinsed with TBST and diluted lysate was applied to plates for 1 h. Following additional rinses, wells were treated with HRP-conjugated anti-Centyrin antibody (PAB25) for 1 h and then assayed with POD (Roche). Centyrins with signals at least 10-fold above background were selected for further analysis.

### Size Exclusion Chromatography Analysis

Size exclusion chromatography was used to determine the aggregation state of PSMA binding Centyrins. Aliquots (10 μL) of each purified Centyrin were injected onto a Superdex 75 5/150 column (GE Healthcare) at a flow rate of 0.3 mL/min in a mobile phase of PBS pH 7.4. Elution from the column was monitored by absorbance at 280 nm. Wild type Tencon was included in each run as a control. Agilent ChemStation software (Rev. B. 04.02) was used to analyze the elution profiles. Only those proteins with elution profiles similar to that of wild type protein in the same run were considered for further characterization.

### High-Throughput Expression, Conjugation and Purification of Centyrins

Isolated clones from unique hits identified by biochemical binding ELISA were combined into a single hit plate for growth in 96-well block plates; clones grew in 1 mL cultures (LB media supplemented with kanamycin for selection) at 37° C. overnight with shaking. For protein expression in 96-block plates, 1 mL TB media supplemented with kanamycin was inoculated with 50 uL of the overnight culture and grown at 37° C. with continual shaking at 300 rpm until OD600=0.6-1. Once the target OD was reached, protein expression was induced with addition of IPTG to 1 mM; plates were transferred to 30° C. (300 rpm) for overnight growth. Overnight cultures were centrifuged to harvest the cells; bacterial pellets were stored at −80° C. until ready for use. Both positive and negative controls were included in replicate on every plate.

For conjugation to the sortase tag, bacterial pellets were thawed, resuspended and lysed in BugBusterHT (EMD Catalog #70922) supplemented with recombinant human lysozyme (EMD, Catalog #71110). Lysis proceeded at room temperature with gentle agitation, after which the plate was transferred to a 42° C. to precipitate host proteins. Debris was pelleted by centrifugation, and supernatants were transferred to a new block plate for sortase-catalyzed labeling. A master mix containing Gly3-vc-MMAF (Concortis), tagless SortaseA, and sortase buffer (Tris, sodium chloride, and calcium chloride) was prepared at a 2× concentration and added in equal volume to the lysate supernatants. The labeling reaction proceeded for two hours at room temperature, after which proteins were purified using a Ni-NTA multi-trap HP plate (GE Catalog #28-4009-89). Protein conjugates were recovered by step elution with imidazole-containing elution buffer (50 mM Tris pH7.5, 500 mM NaCl, 250 mM imidazole), filter sterilized and used directly for cell based cytotoxicity assays.

High-Throughput Cytotoxicity Assay of Centyrin-Drug Conjugates 96-well black tissue culture-coated plates (BD/Corning Catalog #353219) were seeded with LNCaP FGC cells (ATCC, Catalog #CRL-1740) at a density of 10,000 cells/well in assay media (phenol red-free RPMI (Life Technologies Catalog #11835-030) supplemented with 5% fetal bovine serum). Seeded plates were incubated overnight at 37° C. with 5% CO2 to allow for cell attachment. Twenty-four hours later, CDCs were diluted in assay media (1:100, 1:300, 1:1000, or 1:3000) and applied directly to LNCaP cells. LNCaP cells then incubated at 37° C., 5% CO2 for 66-72 h. Cell toxicity was assessed using CellTiter-Glo reagent (Promega, Catalog #G7571); 100 μL prepared reagent was added directly to treated wells and incubated for ten minutes with gentle shaking, protected from light. Luminescence was measured using a SpectraMax M5 plate reader. Values were normalized to untreated controls and selected for further analysis if more than 50% toxicity was achieved.

### Example 4: Characterization of Anti-PSMA Centyrins

### Large-Scale Expression and Purification

Gene sequences encoding Centyrin mutants were discovered through panning and cloned into the pET15b vector for expression under the T7 promoter or produced by DNA2.0 and subcloned into pJexpress401 vector (DNA2.0) for expression under the T5 promoter. The resulting plasmids were transformed into E. coli BL21 Gold (Agilent) or BL21DE3 Gold (Agilent) for expression. A single colony was picked and grown in Luria Broth (Teknova) supplemented with kanamycin and incubated 18 h at 37° C. 250 RPM. One liter Terrific Broth (Teknova), supplemented with kanamycin, was inoculated from these subcultures and grown at 37° C. for 4 h while shaking. Protein expression was induced with 1 mM IPTG, once the optical density at the absorption of 600 nm reached 1.0. The protein was expressed for 4 h at 37° C. or 18 h at 30° C. Cells were harvested by centrifugation at 6000 g and stored at −20 C until purification. The frozen cell pellet (˜15-25 g) was thawed for 30 min at room temperature and suspended in BugBusterHT protein extraction reagent (EMD Millipore) supplemented with 0.2 mg/ml recombinant lysozyme (Sigma) at 5 ml per gram of cell paste and incubated for 1 h on a shaker at room temperature. The lysate was clarified by centrifugation at 74 600 g for 25 min. The supernatant was applied onto a 5 ml Qiagen Ni-NTA cartridge immersed in ice at a flow rate of 4 ml/min using an AKTA AVANT chromatography system. All other Ni-NTA chromatography steps were performed at flow rate 5 ml/min. The Ni-NTA column was equilibrated in 25.0 ml of 50 mM Tris-HCl buffer, pH 7.0 containing 0.5 M NaCl and 10 mM imidazole (Buffer A). After loading, the column was washed with 100 ml of Buffer A, followed by 100 ml of 50 mM Tris-HCl buffer, pH7.0 containing 10 mM imidazole, 1% CHAPS and 1% n-octyl-β-D-glucopyranoside detergents, and 100 ml Buffer A. The protein was eluted with Buffer A supplemented with 250 mM imidazole and loaded on a preparative gel-filtration column, TSK Gel G3000SW 21.5×600 mm (Tosoh) equilibrated in PBS (Gibco). The gel-filtration chromatography was performed at room temperature in PBS at flow rate 10 ml/min using an AKTA-AVANT chromatography system.

### Determination of Thermal Stability

Thermal stability was measured by capillary DSC. Each sample was diluted in PBS pH 7.4 to a concentration of 1 mg/ml. Melting temperatures were measured for these samples using a VP-DSC instrument equipped with an autosampler (MicroCal, LLC). Samples were heated from 10 to 95° C. or 100° C. at a rate of 1° C. per minute. A buffer only scan was completed between each sample scan in order to calculate a baseline for integration. Data were fit to a two-state unfolding model following subtraction of the buffer only signal. Reversibility of thermal denaturation was determined by repeating the scan for each sample without removing it from the cell.

### Selective Cytotoxicity of Anti-PSMA Centyrin Drug Conjugates on PSMA+ Cells

Centyrins were conjugated to vc-MMAF through either cysteine-maleimide chemistry or using the sortase reaction described above. Cytotoxicity of Centyrin-vcMMAF conjugates was assessed in LNCaP, VCAP, MDA-PC-2B, and PC3 cells in vitro. Cells were plated in 96 well black plates for 24 h and then treated with variable doses of Centyrin-vcMMAF conjugates. Cells were allowed to incubate with Centyrin drug conjugates (CDCs) for 66-72 h. CellTiterGlo was used to assess toxicity, as described above. Luminescence values were imported into Excel, from which they were copied and pasted into Prism for graphical analysis. Data were transformed using X=Log(x), then analyzed using nonlinear regression, applying a 3-parameter model to determine IC₅₀.

Table 6 summarizes the unique hits identified through panning, spanning multiple sequence families. Centyrins exhibited thermal stabilities between 55° to 85° C. and were cytotoxic to LNCaP cells when conjugated to vcMMAF, with IC₅₀ values from 22.6-0.38 nM.

### Example 5: Characterization of Anti-PSMA Centyrins

### Large-Scale Expression and Purification

Gene sequences encoding Centyrin mutants were discovered through panning and cloned into the pET15b vector for expression under the T7 promoter or produced by DNA2.0 and subcloned into pJexpress401 vector (DNA2.0) for expression under the T5 promoter. The resulting plasmids were transformed into E. coli BL21 Gold (Agilent) or BL21DE3 Gold (Agilent) for expression. A single colony was picked and grown in Luria Broth (Teknova) supplemented with kanamycin and incubated 18 h at 37° C. 250 RPM. One liter Terrific Broth (Teknova), supplemented with kanamycin, was inoculated from these subcultures and grown at 37° C. for 4 h while shaking. Protein expression was induced with 1 mM IPTG, once the optical density at the absorption of 600 nm reached 1.0. The protein was expressed for 4 h at 37° C. or 18 h at 30° C. Cells were harvested by centrifugation at 6000 g and stored at −20 C until purification. The frozen cell pellet (˜15-25 g) was thawed for 30 min at room temperature and suspended in BugBusterHT protein extraction reagent (EMD Millipore) supplemented with 0.2 mg/ml recombinant lysozyme (Sigma) at 5 ml per gram of cell paste and incubated for 1 h on a shaker at room temperature. The lysate was clarified by centrifugation at 74 600 g for 25 min. The supernatant was applied onto a 5 ml Qiagen Ni-NTA cartridge immersed in ice at a flow rate of 4 ml/min using an AKTA AVANT chromatography system. All other Ni-NTA chromatography steps were performed at flow rate 5 ml/min. The Ni-NTA column was equilibrated in 25.0 ml of 50 mM Tris-HCl buffer, pH 7.0 containing 0.5 M NaCl and 10 mM imidazole (Buffer A). After loading, the column was washed with 100 ml of Buffer A, followed by 100 ml of 50 mM Tris-HCl buffer, pH7.0 containing 10 mM imidazole, 1% CHAPS and 1% n-octyl-β-D-glucopyranoside detergents, and 100 ml Buffer A. The protein was eluted with Buffer A supplemented with 250 mM imidazole and loaded on a preparative gel-filtration column, TSK Gel G3000SW 21.5×600 mm (Tosoh) equilibrated in PBS (Gibco). The gel-filtration chromatography was performed at room temperature in PBS at flow rate 10 ml/min using an AKTA-AVANT chromatography system.

### Determination of Thermal Stability

Thermal stability was measured by capillary DSC. Each sample was diluted in PBS pH 7.4 to a concentration of 1 mg/ml. Melting temperatures were measured for these samples using a VP-DSC instrument equipped with an autosampler (MicroCal, LLC). Samples were heated from 10 to 95° C. or 100° C. at a rate of 1° C. per minute. A buffer only scan was completed between each sample scan in order to calculate a baseline for integration. Data were fit to a two-state unfolding model following subtraction of the buffer only signal. Reversibility of thermal denaturation was determined by repeating the scan for each sample without removing it from the cell.

### Selective Cytotoxicity of Anti-PSMA Centyrin Drug Conjugates on PSMA+ Cells

Centyrins were conjugated to vc-MMAF through either cysteine-maleimide chemistry or using the sortase reaction described above. Cytotoxicity of Centyrin-vcMMAF conjugates was assessed in LNCaP, VCAP, MDA-PC-2B, and PC3 cells in vitro. Cells were plated in 96 well black plates for 24 h and then treated with variable doses of Centyrin-vcMMAF conjugates. Cells were allowed to incubate with Centyrin drug conjugates (CDCs) for 66-72 h. CellTiterGlo was used to assess toxicity, as described above. Luminescence values were imported into Excel, from which they were copied and pasted into Prism for graphical analysis. Data were transformed using X=Log(x), then analyzed using nonlinear regression, applying a 3-parameter model to determine IC50.

Table 5 summarizes the unique hits identified through panning, spanning multiple sequence families. Centyrins exhibited thermal stabilities between 55° to 85° C. and were cytotoxic to LNCaP cells when conjugated to vcMMAF, with IC₅₀ values from 22.6-0.38 nM. Table 6, 7 and 8 shows the BC, C, CD, F and FG loop amino acid sequences of select clones. Table 9 shows the amino acid sequences of the clines.

TABLE 5

| | SEQ ID | Antigen | LNCaP IC₅₀ | Tm |
|---|---|---|---|---|
| Clone ID | NO: | species | (nM) | (□C) |
| P229CR5P819_H11 | 40 | Chimp | 20.7 | 78.1 |
| P258AR6P1071_G03 | 35 | Cyno | 5.8 | 83.1 |
| P258AR6P1070_A05 | 36 | Cyno | 4.6 | 83 |
| P258AR6P1071_F04 | 37 | Cyno | 5.4 | 80.8 |
| P258AR6P1070_F09 | 38 | Cyno | 0.9 | 79.8 |
| P258AR6P1071_D02 | 39 | Cyno | 0.8 | 78.5 |
| P234CR9_H01 | 46 | Cyno | 22.6 | 74.1 |
| P234CR9_A7 | 45 | Cyno | 8.8 | ND |
| P233FR9_H10 | 41 | Chimp | 0.4 | 65.5 |
| P233FR9P1001_D9 | 44 | Chimp | 1.4 | 58.1 |
| P233FR9P1001_B5-5 | 42 | Chimp | 0.5 | 65 |
| P233FR9P1001_H3-1 | 43 | Chimp | 0.4 | 64.5 |

TABLE 6

| Clone ID | | BC loop | | C loop | |
|---|---|---|---|---|---|
| | SEQ | | SEQ | | SEQ |
| | ID | | ID | | ID |
| Clone ID | NO: | Sequence | NO: | Sequence | NO: |
| P229CR5P819_H11 | 40 | DIDEQRDW | 56 | FDSFLIQYQE | 63 |
| P258AR6P1071_G03 | 35 | DIDEQRDW | 56 | FDSFLIQYQE | 63 |
| P258AR6P1070_A05 | 36 | TIDEQRDW | 57 | FDSFLIQYQE | 63 |
| P258AR6P1071_F04 | 37 | VIDEQRDW | 58 | FDSFLIQYQE | 63 |
| P258AR6P1070_F09 | 38 | TIDEQRDW | 57 | FESFLIQYQE | 64 |
| P258AR6P1071_D02 | 39 | AIDEQRDW | 59 | FESFLIQYQE | 64 |
| P234CR9_H01 | 46 | EWWVIPGD | 60 | FDSFLIQYQE | 63 |
| P234CR9_A7 | 45 | GEQFTI | 61 | FDSFLIQYQE | 63 |
| P233FR9_H10 | 41 | TAPDAA | 62 | FDSFAIGYWE | 65 |
| P233FR9P1001_D9 | 44 | TAPDAA | 62 | FDSFPIGYWE | 66 |
| P233FR9P1001_B5- | 42 | TAPDAA | 62 | FDSFTIGYWE | 67 |
| 5 | | | | | |
| P233FR9P1001_H3- | 43 | TAPDAA | 62 | FDSFPIGYWE | 66 |
| 1 | | | | | |

TABLE 7

| Clone ID | | CD loop | | F loop | |
|---|---|---|---|---|---|
| | SEQ | | SEQ | | SEQ |
| | ID | | ID | | ID |
| Clone ID | NO: | Sequence | NO: | Sequence | NO: |
| P229CR5P819_H11 | 40 | SEKVGE | 68 | TEYTVSIYGV | 70 |
| P258AR6P1071_G03 | 35 | SEKVGE | 68 | TEYTVSIYGV | 70 |
| P258AR6P1070_A05 | 36 | SEKVGE | 68 | TEYTVSIYGV | 70 |
| P258AR6P1071_F04 | 37 | SEKVGE | 68 | TEYTVSIYGV | 70 |
| P258AR6P1070_F09 | 38 | SEKVGE | 68 | TEYTVSIYGV | 70 |
| P258AR6P1071_D02 | 39 | SEKVGE | 68 | TEYTVSIYGV | 70 |
| P234CR9_H01 | 46 | SEKVGE | 68 | TEYTVSIYGV | 70 |
| P234CR9_A7 | 45 | SEKVGE | 68 | TEYTVSIYG | 71 |
| P233FR9_H10 | 41 | WDDDGE | 69 | TEYPVYIAGV | 72 |
| P233FR9P1001_D9 | 44 | WDDDGE | 69 | TEYWVYIAGV | 73 |
| P233FR9P1001_B5- | 42 | WDDDGE | 69 | TEYPVYIAGV | 72 |
| 5 | | | | | |
| P233FR9P1001_H3- | 43 | WDDDGE | 69 | TEYHVYIAGV | 74 |
| 1 | | | | | |

TABLE 8

| Clone ID | | FG loop | |
|---|---|---|---|
| | SEQ | | |
| | ID | | SEQ |
| Clone ID | NO: | Sequence | ID NO: |
| P229CR5P819_H11 | 40 | YHVYRSSN | 75 |
| P258AR6P1071_G03 | 35 | YHVYRSN | 76 |
| P258AR6P1070_A05 | 36 | YHVYRSN | 76 |
| P258AR6P1071_F04 | 37 | YHVYRSN | 76 |
| P258AR6P1070_F09 | 38 | YHVYRSN | 76 |
| P258AR6P1071_D02 | 39 | YHVYRSN | 76 |
| P234CR9_H01 | 46 | VNSGQWNDTSN | 77 |
| P234CR9_A7 | 45 | ASGYEWFHAFGSSN | 78 |
| P233FR9_H10 | 41 | KGGQWSF | 79 |
| P233FR9P1001_D9 | 44 | KGGQWSF | 79 |
| P233FR9P1001_B5- | 42 | KGGQWSF | 79 |
| 5 | | | |
| P233FR9P1001_H3- | 43 | KGGQWSF | 79 |
| 1 | | | |

TABLE 9
Clone

| | SEQ | |
|---|---|---|
| | ID | |
| Clone ID | NO: | Sequence |
| P229CR5P819_ | 40 | LPAPKNLVVSRVTEDSARLSWDIDEQRDWFD |
| H11 | | SFLIQYQESEKVGEAIVLTVPGSERSYDLTG |
| | | LKPGTEYTVSIYGVYHVYRSSNPLSAIFTT |
| P258AR6P1071_ | 35 | LPAPKNLVVSRVTEDSARLSWDIDEQRDWFD |
| G03 | | SFLIQYQESEKVGEAIVLTVPGSERSYDLTG |
| | | LKPGTEYTVSIYGVYHVYRSNPLSAIFTT |
| P258AR6P1070_ | 36 | LPAPKNLVVSRVTEDSARLSWTIDEQRDWFD |
| A05 | | SFLIQYQESEKVGEAIVLTVPGSERSYDLTG |
| | | LKPGTEYTVSIYGVYHVYRSNPLSAIFTT |
| P258AR6P1071_ | 37 | LPAPKNLVVSRVTEDSARLSWVIDEQRDWFD |
| F04 | | SFLIQYQESEKVGEAIVLTVPGSERSYDLTG |
| | | LKPGTEYTVSIYGVYHVYRSNPLSAIFTT |
| P258AR6P1070_ | 38 | LPAPKNLVVSRVTEDSARLSWTIDEQRDWFE |
| F09 | | SFLIQYQESEKVGEAIVLTVPGSERSYDLTG |
| | | LKPGTEYTVSIYGVYHVYRSNPLSAIFTT |
| P258AR6P1071_ | 39 | LPAPKNLVVSRVTEDSARLSWAIDEQRDWFE |
| D02 | | SFLIQYQESEKVGEAIVLTVPGSERSYDLTG |
| | | LKPGTEYTVSIYGVYHVYRSNPLSAIFTT |
| P234CR9_H01 | 46 | LPAPKNLVVSRVTEDSARLSWEWWVIPGDFD |
| | | SFLIQYQESEKVGEAIVLTVPGSERSYDLTG |
| | | LKPGTEYTVSIYGVVNSGQWNDTSNPLSAIF |
| | | TT |
| P234CR9_A7 | 45 | LPAPKNLVVSRVTEDSARLSWGEQFTIFDSF |
| | | LIQYQESEKVGEAIVLTVPGSERSYDLTGLK |
| | | PGTEYTVSIYGASGYEWFHAFGSSNPLSAIF |
| | | TT |
| P233FR9_H10 | 41 | LPAPKNLVVSRVTEDSARLSWTAPDAAFDSF |
| | | AIGYWEWDDDGEAIVLTVPGSERSYDLTGLK |
| | | PGTEYPVYIAGVKGGQWSFPLSAIFTT |
| P233FR9P1001_ | 44 | LPAPKNLVVSRVTEDSARLSWTAPDAAFDSF |
| D9 | | PIGYWEWDDDGEAIVLTVPGSERSYDLTGLK |
| | | PGTEYWVYIAGVKGGQWSFPLSAIFTT |
| P233FR9P1001_ | 42 | LPAPKNLVVSRVTEDSARLSWTAPDAAFDSF |
| B5-5 | | TIGYWEWDDDGEAIVLTVPGSERSYDLTGLK |
| | | PGTEYPVYIAGVKGGQWSFPLSAIFTT |
| P233FR9P1001_ | 43 | LPAPKNLVVSRVTEDSARLSWTAPDAAFDSF |
| H3-1 | | PIGYWEWDDDGEAIVLTVPGSERSYDLTGLK |
| | | PGTEYHVYIAGVKGGQWSFPLSAIFTT |

Select centyrin drug conjugates were tested across a panel of cell lines. Table 10 shows the IC₅₀ values for several centyrins conjugated to vcMMAF. Data represent averages between one and nine curve fits. Data are presented as average ±SEM. CDCs were most potent in LNCaP cells, a line known to express high levels of PSMA. CDCs were also active in MDA-PCA-2B and VCAP cells, prostate cancer lines with lower levels of PSMA. No activity was observed in PC3 cells, a PSMA negative cell line, demonstrating selectivity.

TABLE 10

| | | Cytotoxicity Assays of | | | |
|---|---|---|---|---|---|
| | | Centyrin-Drug-Conjugates | | | |
| | | LNCaP | MDA-PCA- | VCAP | PC3 |
| | SEQ | cells | 2B cells | cells | cells |
| | ID | IC₅₀ | IC₅₀ | IC₅₀ | IC₅₀ |
| Clone | NO: | (nM) | (nM) | (nM) | (nM) |
| P233FR9P1001- | 43 | 0.4 | 4.6 ± | 15.2 ± | >500 |
| H3-1 | | | 1.2 | 1.0 | |
| P234CR9_H01 | 46 | 22.6 | 150.8 ± | 401.0 ± | >500 |
| | | | 4.4 | 130.0 | |
| P233FR9_H10 | 41 | 0.5 ± | 5.8 ± | 25.9 ± | >500 |
| | | 0.1 | 2.3 | 15.0 | |
| P229CR5P819_H11 | 40 | 9.3 ± | 106.8 ± | 231.0 ± | >500 |
| | | 1.9 | 13.6 | 38.0 | |

### Example 6: Engineering of Anti-PSMA Centyrins

### Cysteine Scan

Genes encoding anti-PSMA Centyrin, P233FR9_10 with cysteine residues introduced at various positions in the protein were obtained from DNA2.0 and used to express and purify proteins as described above. The resulting centyrins were evaluated for thermal stability (with and without vcMMAF conjugate) and LNCaP cytotoxicity, as described above. Results are summarized in Table 11.

TABLE 11

| | | | Thermal | Thermal | LNCaP |
|---|---|---|---|---|---|
| | | | stability | Stability | cyto- |
| | SEQ ID | Cysteine | (NEM | (vcMMAF | toxicity |
| Clone ID | NO: | location* | capped) | conjugated) | (IC₅₀; nM) |
| P233FR9_H10 | 47 | c- | TBD | TBD | ND |
| (c-term) | | terminal | | | |
| P233FR9_H10 | 51 | K62 | 56.91 | 54.03 | 0.69 |
| (K62C) | | | | | |
| P233FR9_H10 | 50 | R11 | 65.72 | 63.58 | 0.40 |
| (R11C) | | | | | |
| P233FR9_H10 | 49 | E53 | 66.75 | 65.98 | 0.66 |
| (E53C) | | | | | |

*Residue numbering according to SEQ ID NO: 41

### Example 7: Imaging Biodistribution of Untargeted Centyrins

A centyrin with no specific binding to a target antigen engineered to contain a cysteine at position 62 was conjugated to DOTA and then a zirconium-89 radioisotope at IsoTherapeutics Group, LLC (Angleton, Tex.). Castrated male NSG mice were (Jackson laboratories) were anesthetized with 1.5% isoflurane and imaged in a Siemens Inveon microPET/CT. Mice were administered approximately 0.2 mCi [89Zr] Centyrin (SEQ ID 51) via tail vein injection (made up to a 1 mg/kg dose with cold Centyrin) and imaged continuously for the first 60 minutes, and then at 3, 6 and 24 hrs post injection of the Centyrin.

Three-dimensional PET images were reconstructed using a 2D ordered-subsets expectation maximization algorithm (Siemens Healthcare, Knoxville, Tenn.) into a 768×768×512 tomographic volume, with voxel size 0.107 mm×0.107 mm×0.107 mm Images were processed and analyzed using PMOD v3.0 software (PMOD Technologies, Zurich, Switzerland). A cylinder of known activity was scanned in the PET scanner to provide a cross-calibration between injected dose measured by the dose calibrator, and counts per voxel in the PET images. Each PET image was co-registered to the CT image, to provide anatomical reference, using PMOD image fusion software. Regions of interest (ROI) were drawn around every 4th section for each tissue being analyzed. Mean counts per voxel were derived, and converted Percentage injected dose per gram of body weight, and using the correction factor derived from the calibration cylinder of known activity. All measures of radioactivity were corrected for decay, using the known half-life of Zr-89 (78.41 hours).

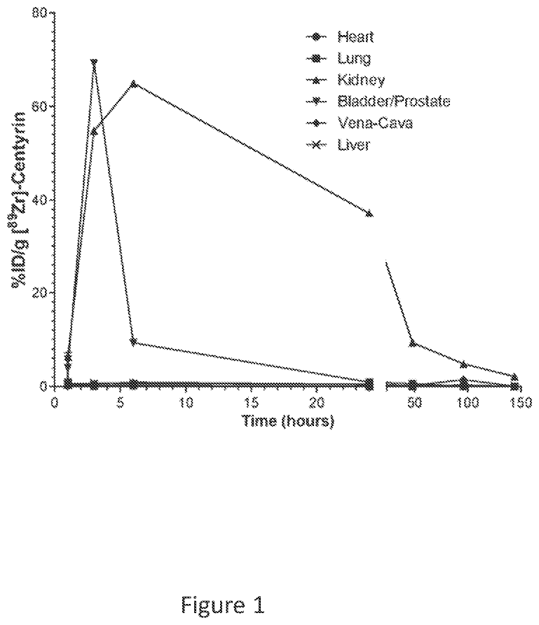

FIG. 1 shows the tissue distribution of radiolabeled FN3 domain over time. Rapid accumulation in the kidney and bladder is observed, with only limited accumulation in the liver, suggesting that Centyrins are cleared through the kidneys.

### Example 8: Crystal Structure of Anti-PSMA P233FR9-H10 in Complex with Cyno PSMA

The His-tagged P233FR9-H10 centyrin (called herein as H10 centyrin) was expressed in E. coli and purified using affinity and size-exclusion chromatography. The centyrin was received in dPBS, pH 7.2.

The cynomolgus PSMA extracellular domain as a C-terminal fusion to the huIgG1 Fc domain was expressed in GnTI⁻ cells and purified by affinity and size-exclusion chromatography. The fusion protein was received in dPBS, 0.5 mM ZnCl₂, pH 7.2. Then, the Fc domain was removed with a Prescission protease treatment followed by affinity and size-exclusion chromatography. The isolated cynomolgus PSMA (cynoPSMA) extracellular domain was stored in dPBS, 0.5 mM ZnCl₂, pH 7.2.

The H10 centyrin/cynoPSMA complex was prepared by mixing cynoPSMA with H10 centyrin at a molar ratio of 1:3 (excess centyrin) while dialyzing for 48 h at 4° C. against 20 mM Hepes pH 7.0, 0.5 mM ZnCl₂. The complex was then eluted from a monoS column with a gradient of 48-68 mM NaCl, 20 mM Hepes pH 7.5, 10% glycerol and concentrated to 3.4 mg/mL. Crystals suitable for X-diffraction were obtained from 25% PEG 3 kDa, 0.2 M NH₄Cl, 0.1 M Na Acetate pH 4.5 using the sitting drop vapor-diffusion method at 20° C.

For X-ray data collection, the crystal was soaked for a few seconds in a cryo-protectant solution containing mother liquor supplemented with 20% glycerol, and then frozen in liquid nitrogen. X-ray diffraction data were collected with a Dectris Pilatus 6M Pixel Array detector at the beamline 17-ID of the Advanced Photon Source (APS) at Argonne National Laboratory. Diffraction data were processed with the program HKL2000 (Otwinowski & Minor, 1997). X-ray data statistics are given in Table 12.

The structure was solved by molecular replacement (MR) with Phaser (Read, 2001). The search models for MR were the crystal structures of human PSMA (PDB code 2C6G) and the structure of P114AR7P94-A3 W33A centyrin. The structures were refined with PHENIX (Adams et al, 2004) and model adjustments were carried out using COOT (Emsley & Cowtan, 2004). All other crystallographic calculations were performed with the CCP4 suite of programs (CCP4, 1994). All molecular graphics were generated with PyMol (DeLano, 2002). The structure refinement statistics are given in Table 12.

TABLE 12

PS42

Crystal data

| | |
|---|---|
| Crystallization solution | |
| 0.1M Buffer | Acetate pH 4.5 |
| Precipitant | 25% PEG 3 kDa |
| Additive | 0.2M NH₄Cl |
| Space group | P2₁2₁2 |
| Complex/asym. unit | 2 |
| Unit cell | |
| a (Å) | 84.0 |
| b (Å) | 109.9 |
| c (Å) | 261.6 |
| Vₘ (Å³/Da) | 3.32 |
| Solvent content (%) | 63 |

X-ray data*

| | |
|---|---|
| Resolution (Å) | 50.00-2.80 |
| High Resolution Shell (Å) | (2.85-2.80) |
| Measured reflections | 335,467 |
| Unique reflections | 57,166 |
| Completeness (%) | 93.2 (69.3) |
| Redundancy | 5.9 (4.4) |
| Rsym (%) | 25.1 (64.1) |
| <I/σ> | 6.4 (1.6) |

Refinement

| | |
|---|---|
| Resolution (Å) | 40.0-2.8 |
| Number of reflections | 57,063 |
| Number of all atoms | 12,330 |
| Number of waters | 8 |
| Rfactor (%) | 25.14 |
| Rfree(%) | 31.28 |
| RMSD | |
| bond lengths (Å) | 0.003 |
| bond angles (°) | 0.998 |
| Average B-factor (Å²) | 78.9 |
| Ramachandran Plot | |
| favored region (%) | 94.9 |
| allowed region (%) | 4.8 |
| outliers (%) | 0.3 |

*Values for high resolution shell are in parenthesis.

The structure of the homodimeric cynoPSMA includes residues 57-750, corresponding to the protease (residues 57-116 and 352-590), apical (residues 117-351) and helical (residues 591-750) domains, and eight of eleven possible N-linked glycans (in Asn-76, -121, -140, -195, -459, -476, -613, and -638) per dimer subunit. The cynoPSMA active site is located at the interface between the three domains and it contains two zinc atoms coordinated by histidine (H377 and H553) and glutamate/aspartate (D387, catalytic E424, E425, and D453) residues and a water molecule. The H10 centyrin (SEQ ID NO: 41) structure contains residues 2-92. H10 residues are numbered sequentially according to SEQ ID NO: 41. cynoPSMA residues are numbered according to the full length cyno PSMA sequence of SEQ ID NO: 141. The mature cynoPSMA (without signal peptide) starts at residue 44 of SEQ ID NO: 141.

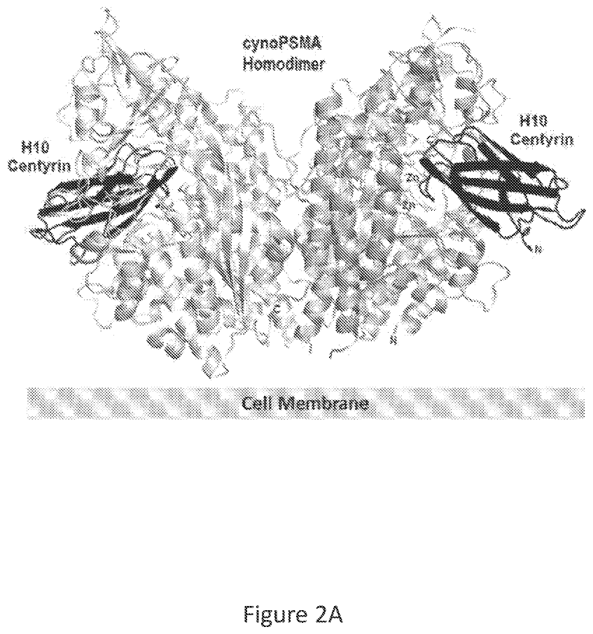

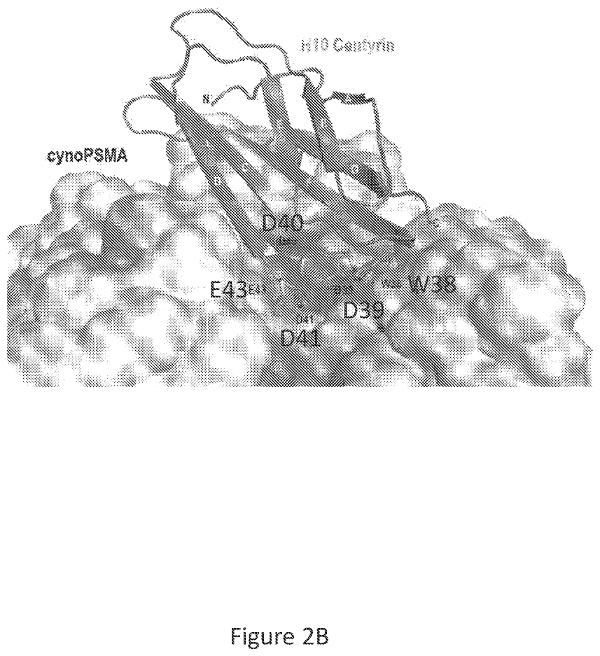

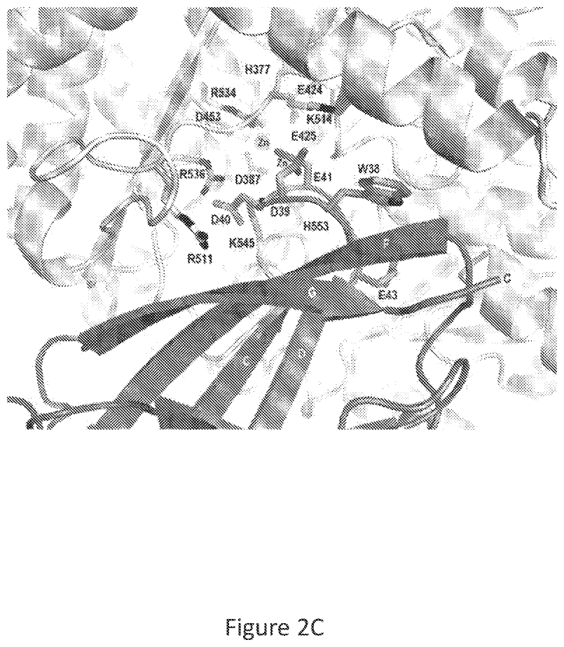

There is one cynoPSMA homodimer in the asymmetric unit with one H10 centyrin (SEQ ID NO: 41) bound to each PSMA subunit (FIG. 2A). The two centyrin/PSMA complexes are structurally very similar as indicated by the root mean square deviation (r.m.s.d.) of 0.72 Å for the superposition of all equivalent atoms in the PSMA subunits. Also, there is a high degree of structural similarity between human and cynomolgus PSMA and absence of large conformational changes induced by the centyrin binding, as indicated by a r.m.s.d of 0.5 Å for the Cα atom superposition between the cynoPSMA molecule in the centyrin complex and unbound human PSMA (PDB code 2OOT, structure at 1.6 Å resolution). An interesting feature is that the loop region 541-547 is visible only in the cynomolgus protein due to stabilization of the loop conformation through interactions with the centyrin.

The centyrin/PSMA combining site is well defined by the 2F_{obs}-F_{calc} electron density map, which allows reliable positioning of the binding residues. Only the interactions between the B and C chains (PSMA and centyrin chains, respectively) are described in the next section.

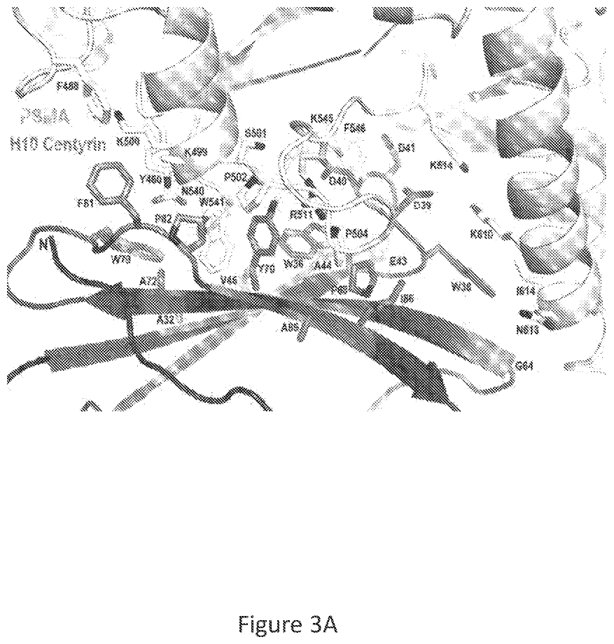

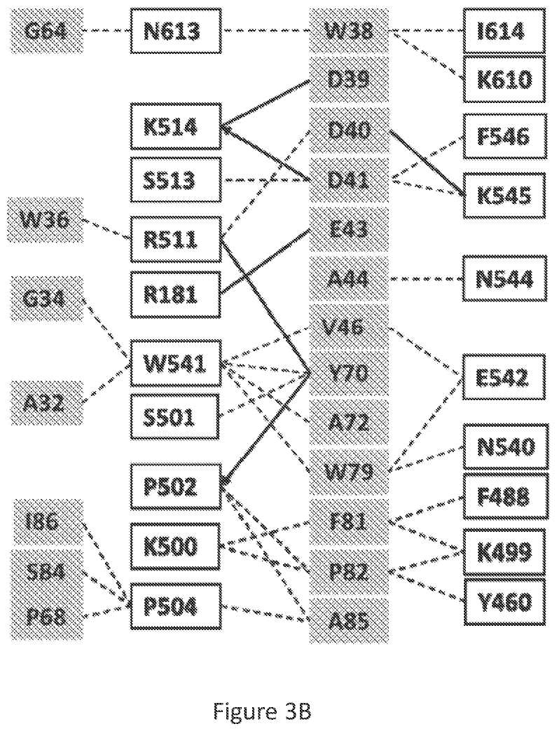

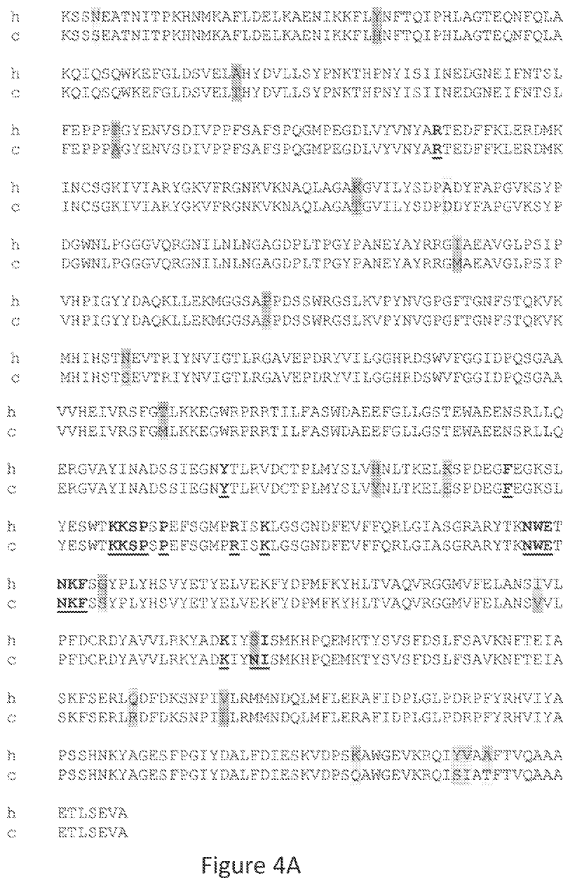

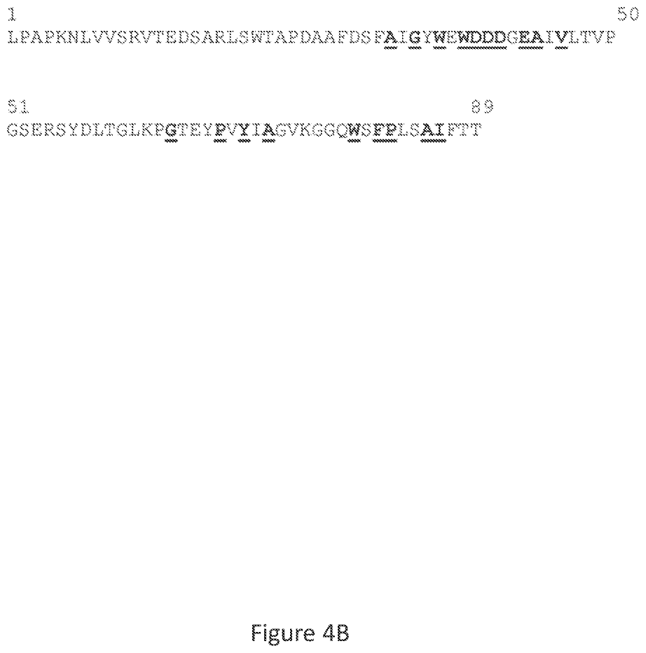

The H10 (SEQ ID NO: 41) centyrin binds to a region near the PSMA active site (FIG. 2A) and covers a cynoPSMA area of about 1,170 Å². Specifically, the centyrin recognizes cynoPSMA residues in the protease (Y460, F488, K499-P502, P504, R511, K514, N540-E542, and N544-F546), apical (residue R181), and helical (residues K610, N613, and I614) domains as shown in FIGS. 3 and 4.

The face of the centyrin four-stranded β-sheet packs onto the PSMA surface with the CD loop deeply inserted into the active site entrance (FIGS. 2B and 2C). Specifically, the H10 centyrin (SEQ ID NO: 41) residues involved in PSMA binding are located in the C (A32 and G34), D (V46), F (G64, P68, Y70, and A72), and G (S84-I86) β-strands and the CD (W36, W38-D41, E43, and A44) and FG loops (W79, F81, and P82). Residues D39, D40, D41, and E43 confer a negative charge to the centyrin CD loop and these residues are inserted into the ˜20 Å deep, positively charged, funnel that leads to the zinc ions in the active site, likely blocking substrate entrance into the funnel and PSMA enzymatic activity (FIGS. 2B and 2C). However, the centyrin does not interact directly with the zinc ions or their coordinating residues.

Conserved PSMA residues W541, Y460, F488, P502 and P504 form an aromatic cluster across the combing site with centyrin residues W36, P68, Y70, W79, F81, and P82 (FIG. 3A). Conserved R511 is in a central location of the combining site and H bonds Y70, a central residue of the centyrin four-stranded β-sheet. FIG. 3B shows a cartoon of the paratope and epitope residues.

Human and cynomolgus PSMA are 97% identical, and, except for a S613N change, all residues interacting with H10 (SEQ ID NO: 41) are conserved between the two species (FIG. 4). The S613N change results in N613 glycosylation in cynoPSMA and the gain of van der Waals contacts between the carbohydrate and centyrin residues E66, 186, T88 (F and G β-strands) that will not be present in the human enzyme.

### Centyrin Residues for Conjugation

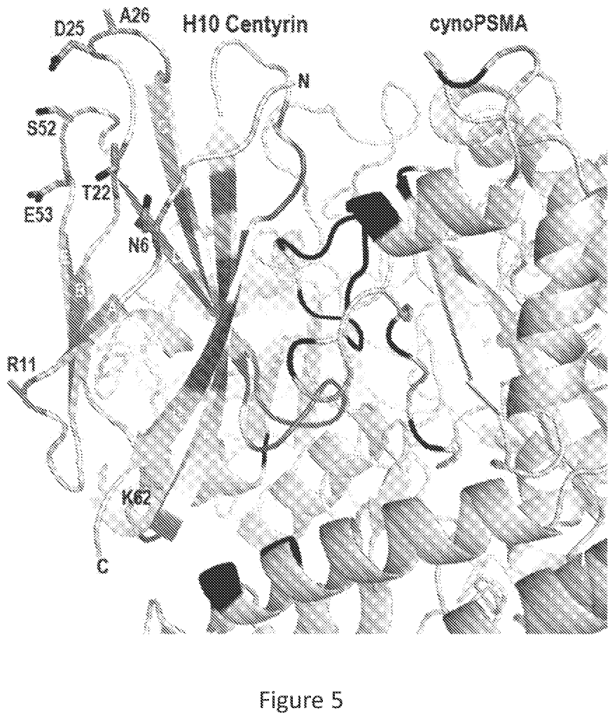

Various H10 centyrin (SEQ ID NO: 41) residues outside the combining site can be modified for conjugation of small molecules (toxic payloads) without disrupting PSMA binding or centyrin fold. Cysteines were already placed and conjugated to payloads at the C-terminus (after the His-tag) and at positions R11, E53, and K62 and all of these variants demonstrated similarly potent cytotoxicity. In addition, residues T22, D25, and A26 in the BC loop, terminal residue N6, and S52 in the DE loop are potentially good sites for mutagenesis followed by chemical conjugation (FIG. 5). These solvent exposed residues are away from the centyrin/PSMA interface and located in structurally flexible regions.

Furthermore, both N- and C-terminal regions are free for fusions with other protein domains. The N-terminus is oriented towards the PSMA protease domain and reachable with a fusion linker, while the also accessible C-terminus goes towards the PSMA helical domain. The optimal linker length to the centyrin fusion partner will depend on the structure of the fusion partner and location of its binding site on the target molecule.

### Mechanism of Action

The H10 centyrin (SEQ ID NO: 41) is a candidate for targeted delivery of payloads (toxic small molecules, nucleic acid, etc.) into prostate cancer cells due to internalization of the centyrin/PSMA complex. Furthermore, the H10 centyrin is a candidate for redirection of immune cells to prostate cancer cells when in a multispecific format.

H10 centyrin (SEQ ID NO: 41) is likely to also inhibit the enzymatic activity of PSMA, which may contribute to decreased cell fitness and survival. The centyrin/cynoPSMA structure shows the centyrin bound to the entrance of the active site, which might prevent substrate interaction with PSMA through steric occlusion and direct competition for the binding site.

### Example 9: Generation of Additional Anti-PSMA Centyrin Variants

Select anti-PSMA centyrins were further engineered to improve properties of the parental centyrins. FN3 domains binding to PSMA were generated using libraries described above, and tested for their binding to PSMA.

Table 13 shows the amino acid sequences of the generated molecules.

| | SEQ ID | |
|---|---|---|
| Clone ID | NO: | Sequence |
| P258AR6P1071_ | 173 | LPAPKNLVVSRVTEDSARLSWAADEQRDWFESFLIQYQESE |
| D02_v1 | | KVGEAIVLTVPGSCRSYDLTGLKPGTEYTVSIYGVYHVYRS |
| | | NPLSAIFTT |
| P258AR6P1071_ | 174 | LPAPKNLVVSRVTEDSARLSWAIAEQRDWFESFLIQYQESE |
| D02_v2 | | KVGEAIVLTVPGSCRSYDLTGLKPGTEYTVSIYGVYHVYRS |
| | | NPLSAIFTT |
| P258AR6P1071_ | 175 | LPAPKNLVVSRVTEDSARLSWAIDAQRDWFESFLIQYQESE |
| D02_v3 | | KVGEAIVLTVPGSCRSYDLTGLKPGTEYTVSIYGVYHVYRS |
| | | NPLSAIFTT |
| P258AR6P1071_ | 176 | LPAPKNLVVSRVTEDSARLSWAIDEARDWFESFLIQYQESE |
| D02_v4 | | KVGEAIVLTVPGSCRSYDLTGLKPGTEYTVSIYGVYHVYRS |
| | | NPLSAIFTT |
| P258AR6P1071_ | 177 | LPAPKNLVVSRVTEDSARLSWAIDEQADWFESFLIQYQESE |
| D02_v5 | | KVGEAIVLTVPGSCRSYDLTGLKPGTEYTVSIYGVYHVYRS |
| | | NPLSAIFTT |
| P258AR6P1071_ | 80 | LPAPKNLVVSRVTEDSARLSWAIDEQRAWFESFLIQYQESE |
| D02_v6 | | KVGEAIVLTVPGSCRSYDLTGLKPGTEYTVSIYGVYHVYRS |
| | | NPLSAIFTT |
| P258AR6P1071_ | 81 | LPAPKNLVVSRVTEDSARLSWAIDEQRDAFESFLIQYQESE |
| D02_v7 | | KVGEAIVLTVPGSCRSYDLTGLKPGTEYTVSIYGVYHVYRS |
| | | NPLSAIFTT |
| P258AR6P1071_ | 82 | LPAPKNLVVSRVTEDSARLSWAIDEQRDWFESFLIQYQESE |
| D02_v8 | | KVGEAIVLTVPGSCRSYDLTGLKPGTEYTVSIYGVAHVYRS |
| | | NPLSAIFTT |
| P258AR6P1071_ | 83 | LPAPKNLVVSRVTEDSARLSWAIDEQRDWFESFLIQYQESE |
| D02_v9 | | KVGEAIVLTVPGSCRSYDLTGLKPGTEYTVSIYGVYAVYRS |
| | | NPLSAIFTT |
| P258AR6P1071_ | 84 | LPAPKNLVVSRVTEDSARLSWAIDEQRDWFESFLIQYQESE |
| D02_v10 | | KVGEAIVLTVPGSCRSYDLTGLKPGTEYTVSIYGVYHAYRS |
| | | NPLSAIFTT |
| P258AR6P1071_ | 85 | LPAPKNLVVSRVTEDSARLSWAIDEQRDWFESFLIQYQESE |
| D02_v11 | | KVGEAIVLTVPGSCRSYDLTGLKPGTEYTVSIYGVYHVARS |
| | | NPLSAIFTT |
| P258AR6P1071_ | 86 | LPAPKNLVVSRVTEDSARLSWAIDEQRDWFESFLIQYQESE |
| D02_v12 | | KVGEAIVLTVPGSCRSYDLTGLKPGTEYTVSIYGVYHVYAS |
| | | NPLSAIFTT |
| P258AR6P1071_ | 87 | LPAPKNLVVSRVTEDSARLSWAIDEQRDWFASFLIQYQESE |
| D02_v13 | | KVGEAIVLTVPGSCRSYDLTGLKPGTEYTVSIYGVYHVYRS |
| | | NPLSAIFTT |
| P258AR6P1071_ | 88 | LPAPKNLVVSRVTEDSARLSWDIDEQRDWFESFLIQYQESE |
| D02_v14 | | KVGEAIVLTVPGSCRSYDLTGLKPGTEYTVSIYGVYHVYRS |
| | | NPLSAIFTT |
| P258AR6P1071_ | 89 | LPAPKNLVVSRVTEDSARLSWAIDEQRDWFDSFLIQYQESE |
| D02_v15 | | KVGEAIVLTVPGSCRSYDLTGLKPGTEYTVSIYGVYHVYRS |
| | | NPLSAIFTT |
| P258AR6P1071_ | 90 | LPAPKNLVVSRVTEDSARLSWAIDEQRDWFESFLIQYQESE |
| D02_v16 | | KVGEAIVLTVPGSCRSYDLTGLKPGTEYTVSIYGVYHVYRS |
| | | SNPLSAIFTT |
| P258AR6P1071_ | 91 | LPAPKNLVVSRVTEDSARLSWDIDEQRDWFDSFLIQYQESE |
| D02_v17 | | KVGEAIVLTVPGSCRSYDLTGLKPGTEYTVSIYGVYHVYRS |
| | | NPLSAIFTT |
| P258AR6P1071_ | 92 | LPAPKNLVVSRVTEDSARLSWDIDEQRDWFESFLIQYQESE |
| D02_v18 | | KVGEAIVLTVPGSCRSYDLTGLKPGTEYTVSIYGVYHVYRS |
| | | SNPLSAIFTT |
| P258AR6P1071_ | 93 | LPAPKNLVVSRVTEDSARLSWAIDEQRDWFDSFLIQYQESE |
| D02_v19 | | KVGEAIVLTVPGSCRSYDLTGLKPGTEYTVSIYGVYHVYRS |
| | | SNPLSAIFTT |
| P233FR9_ | 94 | LPAPKNLVVSRVTEDSARLSWTAPDAAFDSFAIGYWEWDDD |
| H10_v1 | | GEAIVLTVPGSCRSYDLTGLKPGTEYRVYIAGVKGGQWSFP |
| | | LSAIFTT |
| P233FR9_ | 95 | LPAPKNLVVSRVTEDSARLSWTAPDAAFDSFAIGYWEWDDD |
| H10_v2 | | GEAIVLTVPGSCRSYDLTGLKPGTEYKVYIAGVKGGQWSFP |
| | | LSAIFTT |
| P233FR9_ | 96 | LPAPKNLVVSRVTEDSARLSWTAPDAAFDSFAIGYWEWDDD |
| H10_v3 | | GEAIVLTVPGSCRSYDLTGLKPGTEYEVYIAGVKGGQWSFP |
| | | LSAIFTT |
| P233FR9_ | 97 | LPAPKNLVVSRVTEDSARLSWTAPDAAFDSFAIGYWEWDDD |
| H10_v4 | | GEAIVLTVPGSCRSYDLTGLKPGTEYHVYIAGVKGGQWSFP |
| | | LSAIFTT |
| P233FR9_ | 98 | LPAPKNLVVSRVTEDSARLSWTAPDAAFDSFAIGYWEWDDD |
| H10_v5 | | GEAIVLTVPGSCRSYDLTGLKPGTEYDVYIAGVKGGQWSFP |
| | | LSAIFTT |
| P233FR9_ | 99 | LPAPKNLVVSRVTEDSARLSWTAPDAAFDSFAIGYWEWDDD |
| H10_v6 | | GEAIVLTVPGSCRSYDLTGLKPGTEYAVYIAGVKGGQWSFP |
| | | LSAIFTT |
| P233FR9_ | 100 | LPAPKNLVVSRVTEDSARLSWTAPDAAFDSFAIGYWEWDDD |
| H10_v7 | | GEAIVLTVPGSCRSYDLTGLKPGTEYGVYIAGVKGGQWSFP |
| | | LSAIFTT |
| P233FR9_ | 101 | LPAPKNLVVSRVTEDSARLSWTAPDAAFDSFAIGYWEWDDD |
| H10_v8 | | GEAIVLTVPGSCRSYDLTGLKPGTEYVVYIAGVKGGQWSFP |
| | | LSAIFTT |
| P233FR9_ | 102 | LPAPKNLVVSRVTEDSARLSWTAPDAAFDSFAIGYWEWDDD |
| H10_v9 | | GEAIVLTVPGSCRSYDLTGLKPGTEYLVYIAGVKGGQWSFP |
| | | LSAIFTT |
| P233FR9_ | 103 | LPAPKNLVVSRVTEDSARLSWTAPDAAFDSFAIGYWEWDDD |
| H10_v10 | | GEAIVLTVPGSCRSYDLTGLKPGTEYIVYIAGVKGGQWSFP |
| | | LSAIFTT |
| P233FR9_ | 104 | LPAPKNLVVSRVTEDSARLSWTAPDAAFDSFAIGYWEWDDD |
| H10_v11 | | GEAIVLTVPGSCRSYDLTGLKPGTEYFVYIAGVKGGQWSFP |
| | | LSAIFTT |
| P233FR9_ | 105 | LPAPKNLVVSRVTEDSARLSWTAPDAAFDSFAIGYWEWDDD |
| H10_v12 | | GEAIVLTVPGSCRSYDLTGLKPGTEYWVYIAGVKGGQWSFP |
| | | LSAIFTT |
| P233FR9_ | 106 | LPAPKNLVVSRVTEDSARLSWTAPDAAFDSFAIGYWEWDDD |
| H10_v13 | | GEAIVLTVPGSCRSYDLTGLKPGTEYNVYIAGVKGGQWSFP |
| | | LSAIFTT |
| P233FR9_ | 107 | LPAPKNLVVSRVTEDSARLSWTAPDAAFDSFAIGYWEWDDD |
| H10_v14 | | GEAIVLTVPGSCRSYDLTGLKPGTEYQVYIAGVKGGQWSFP |
| | | LSAIFTT |
| P233FR9_ | 108 | LPAPKNLVVSRVTEDSARLSWTAPDAAFDSFAIGYWEWDDD |
| H10_v15 | | GEAIVLTVPGSCRSYDLTGLKPGTEYSVYIAGVKGGQWSFP |
| | | LSAIFTT |
| P233FR9_ | 109 | LPAPKNLVVSRVTEDSARLSWTAPDAAFDSFAIGYWEWDDD |
| H10_v16 | | GEAIVLTVPGSCRSYDLTGLKPGTEYTVYIAGVKGGQWSFP |
| | | LSAIFTT |
| P233FR9_ | 110 | LPAPKNLVVSRVTEDSARLSWTAPDAAFDSFAIGYWEWDDD |
| H10_v17 | | GEAIVLTVPGSCRSYDLTGLKPGTEYYVYIAGVKGGQWSFP |
| | | LSAIFTT |
| P233FR9_ | 111 | LPAPKNLVVSRVTEDSARLSWTAPDAAFDSFAIAYWEWDDD |
| H10_v18 | | GEAIVLTVPGSCRSYDLTGLKPGTEYPVYIAGVKGGQWSFP |
| | | LSAIFTT |
| P233FR9_ | 112 | LPAPKNLVVSRVTEDSARLSWTAPDAAFDSFAISYWEWDDD |
| H10_v19 | | GEAIVLTVPGSCRSYDLTGLKPGTEYPVYIAGVKGGQWSFP |
| | | LSAIFTT |
| P233FR9_ | 113 | LPAPKNLVVSRVTEDSARLSWTAPDAAFDSFAIGYWEWDTD |
| H10_v20 | | GEAIVLTVPGSCRSYDLTGLKPGTEYPVYIAGVKGGQWSFP |
| | | LSAIFTT |
| P233FR9_ | 114 | LPAPKNLVVSRVTEDSARLSWTAPDAAFDSFAIGYWEWDSD |
| H10_v21 | | GEAIVLTVPGSCRSYDLTGLKPGTEYPVYIAGVKGGQWSFP |
| | | LSAIFTT |
| P233FR9_ | 115 | LPAPKNLVVSRVTEDSARLSWTAPDAAFDSFAIGYYEWDDD |
| H10_v22 | | GEAIVLTVPGSCRSYDLTGLKPGTEYPVYIAGVKGGQWSFP |
| | | LSAIFTT |
| P233FR9_ | 116 | LPAPKNLVVSRVTEDSARLSWTAPDAAFDSFAIGYFEWDDD |
| H10_v23 | | GEAIVLTVPGSCRSYDLTGLKPGTEYPVYIAGVKGGQWSFP |
| | | LSAIFTT |
| P233FR9_ | 117 | LPAPKNLVVSRVTEDSARLSWTAPDAAFDSFAIGYLEWDDD |
| H10_v24 | | GEAIVLTVPGSCRSYDLTGLKPGTEYPVYIAGVKGGQWSFP |
| | | LSAIFTT |
| P233FR9_ | 118 | LPAPKNLVVSRVTEDSARLSWTAPDAAFDSFAIGYWEYDDD |
| H10_v25 | | GEAIVLTVPGSCRSYDLTGLKPGTEYPVYIAGVKGGQWSFP |
| | | LSAIFTT |
| P233FR9_ | 119 | LPAPKNLVVSRVTEDSARLSWTAPDAAFDSFAIGYWEFDDD |
| H10_v26 | | GEAIVLTVPGSCRSYDLTGLKPGTEYPVYIAGVKGGQWSFP |
| | | LSAIFTT |
| P233FR9_ | 120 | LPAPKNLVVSRVTEDSARLSWTAPDAAFDSFAIGYWELDDD |
| H10_v27 | | GEAIVLTVPGSCRSYDLTGLKPGTEYPVYIAGVKGGQWSFP |
| | | LSAIFTT |
| P233FR9_ | 121 | LPAPKNLVVSRVTEDSARLSWTAPDAAFDSFAIGYWEWDDD |
| H10_v28 | | GEAIVLTVPGSCRSYDLTGLKPGTEYPVYIAGVKGGQYSFP |
| | | LSAIFTT |
| P233FR9_ | 122 | LPAPKNLVVSRVTEDSARLSWTAPDAAFDSFAIGYWEWDDD |
| H10_v29 | | GEAIVLTVPGSCRSYDLTGLKPGTEYPVYIAGVKGGQFSFP |
| | | LSAIFTT |
| P233FR9_ | 123 | LPAPKNLVVSRVTEDSARLSWTAPDAAFDSFAIGYWEWDDD |
| H10_v30 | | GEAIVLTVPGSCRSYDLTGLKPGTEYPVYIAGVKGGQLSFP |
| | | LSAIFTT |
| P233FR9P1001- | 124 | LPAPKNLVVSRVTEDSARLSWTAPDAAFDSFRIGYWEWDDD |
| H3-1_v1 | | GEAIVLTVPGSCRSYDLTGLKPGTEYHVYIAGVKGGQWSFP |
| | | LSAIFTT |
| P233FR9P1001- | 125 | LPAPKNLVVSRVTEDSARLSWTAPDAAFDSFKIGYWEWDDD |
| H3-1_v2 | | GEAIVLTVPGSCRSYDLTGLKPGTEYHVYIAGVKGGQWSFP |
| | | LSAIFTT |
| P233FR9P1001- | 126 | LPAPKNLVVSRVTEDSARLSWTAPDAAFDSFEIGYWEWDDD |
| H3-1_v3 | | GEAIVLTVPGSCRSYDLTGLKPGTEYHVYIAGVKGGQWSFP |
| | | LSAIFTT |
| P233FR9P1001- | 127 | LPAPKNLVVSRVTEDSARLSWTAPDAAFDSFHIGYWEWDDD |
| H3-1_v4 | | GEAIVLTVPGSCRSYDLTGLKPGTEYHVYIAGVKGGQWSFP |
| | | LSAIFTT |
| P233FR9P1001- | 128 | LPAPKNLVVSRVTEDSARLSWTAPDAAFDSFDIGYWEWDDD |
| H3-1_v5 | | GEAIVLTVPGSCRSYDLTGLKPGTEYHVYIAGVKGGQWSFP |
| | | LSAIFTT |
| P233FR9P1001- | 129 | LPAPKNLVVSRVTEDSARLSWTAPDAAFDSFAIGYWEWDDD |
| H3-1_v6 | | GEAIVLTVPGSCRSYDLTGLKPGTEYHVYIAGVKGGQWSFP |
| | | LSAIFTT |
| P233FR9P1001- | 130 | LPAPKNLVVSRVTEDSARLSWTAPDAAFDSFGIGYWEWDDD |
| H3-1_v7 | | GEAIVLTVPGSCRSYDLTGLKPGTEYHVYIAGVKGGQWSFP |
| | | LSAIFTT |
| P233FR9P1001- | 131 | LPAPKNLVVSRVTEDSARLSWTAPDAAFDSFVIGYWEWDDD |
| H3-1_v8 | | GEAIVLTVPGSCRSYDLTGLKPGTEYHVYIAGVKGGQWSFP |
| | | LSAIFTT |
| P233FR9P1001- | 132 | LPAPKNLVVSRVTEDSARLSWTAPDAAFDSFLIGYWEWDDD |
| H3-1_v9 | | GEAIVLTVPGSCRSYDLTGLKPGTEYHVYIAGVKGGQWSFP |
| | | LSAIFTT |
| P233FR9P1001- | 133 | LPAPKNLVVSRVTEDSARLSWTAPDAAFDSFIIGYWEWDDD |
| H3-1_v10 | | GEAIVLTVPGSCRSYDLTGLKPGTEYHVYIAGVKGGQWSFP |
| | | LSAIFTT |
| P233FR9P1001- | 134 | LPAPKNLVVSRVTEDSARLSWTAPDAAFDSFFIGYWEWDDD |
| H3-1_v11 | | GEAIVLTVPGSCRSYDLTGLKPGTEYHVYIAGVKGGQWSFP |
| | | LSAIFTT |
| P233FR9P1001- | 135 | LPAPKNLVVSRVTEDSARLSWTAPDAAFDSFWIGYWEWDDD |
| H3-1_v12 | | GEAIVLTVPGSCRSYDLTGLKPGTEYHVYIAGVKGGQWSFP |
| | | LSAIFTT |
| P233FR9P1001- | 136 | LPAPKNLVVSRVTEDSARLSWTAPDAAFDSFNIGYWEWDDD |
| H3-1_v13 | | GEAIVLTVPGSCRSYDLTGLKPGTEYHVYIAGVKGGQWSFP |
| | | LSAIFTT |
| P233FR9P1001- | 137 | LPAPKNLVVSRVTEDSARLSWTAPDAAFDSFQIGYWEWDDD |
| H3-1_v14 | | GEAIVLTVPGSCRSYDLTGLKPGTEYHVYIAGVKGGQWSFP |
| | | LSAIFTT |
| P233FR9P1001- | 138 | LPAPKNLVVSRVTEDSARLSWTAPDAAFDSFSIGYWEWDDD |
| H3-1_v15 | | GEAIVLTVPGSCRSYDLTGLKPGTEYHVYIAGVKGGQWSFP |
| | | LSAIFTT |
| P233FR9P1001- | 139 | LPAPKNLVVSRVTEDSARLSWTAPDAAFDSFTIGYWEWDDD |
| H3-1_v16 | | GEAIVLTVPGSCRSYDLTGLKPGTEYHVYIAGVKGGQWSFP |
| | | LSAIFTT |
| P233FR9P1001- | 140 | LPAPKNLVVSRVTEDSARLSWTAPDAAFDSFYIGYWEWDDD |
| H3-1_v17 | | GEAIVLTVPGSCRSYDLTGLKPGTEYHVYIAGVKGGQWSFP |
| | | LSAIFTT |

### Example 10: Detection of PSMA Expression on Tumor Cells Using Anti PSMA Centyrin Conjugated to Fluorescent Dye

This example shows the detection of PSMA present on cells with anti PSMA centyrin conjugated to a fluorescent dye. C-terminally His-tagged anti PSMA Centyrin P233FR9_H10 (SEQ ID NO: 49) with a free cysteine at amino acid 53 was conjugated to R-phycoerythrin (PE) (Prozyme catalog #PB31). The PE was activated using sulfo-SMCC (Pierce catalog #22122) for 60 min, and activated PE was separated from free sulfo-SMCC by gel filtration chromatography using Sephadex G25 and PBS/EDTA buffer. The centyrin was reduced using TCEP (Sigma, cat. #646547) for 30 min. The reduced centyrin was separated from free TCEP by gel filtration chromatography using Sephadex G25 and PBS/EDTA buffer. The activated R-PE was covalently coupled to the reduced centyrin for 90 min followed by quenching with N-Ethylmaleimide (Sigma catalog #04260) for 20 min. The “PE-conjugated Centyrin” was purified by size-exclusion chromatography (SEC) using a Tosoh TSKgel G3000SW column in 100 mM sodium phosphate, 100 mM sodium sulfate, 0.05% sodium azide, pH 6.5 on an AKTA explorer FPLC (General Electric).

The PE-conjugate Centyrin-was tested for sensitivity and specificity using PSMA positive and negative cell lines by flow cytometry and CellSearch Circulating Tumor Cell (CTC) assay. The following prostate cell lines were purchased from ATCC and used to validate the specificity of the anti-PSMA centyrin: LNCaP (high PSMA expression), 22Rv1 (low PSMA expression) and PC3 (no PSMA expression).

### Detection of PSMA on Cell Lines by Flow Cytometry

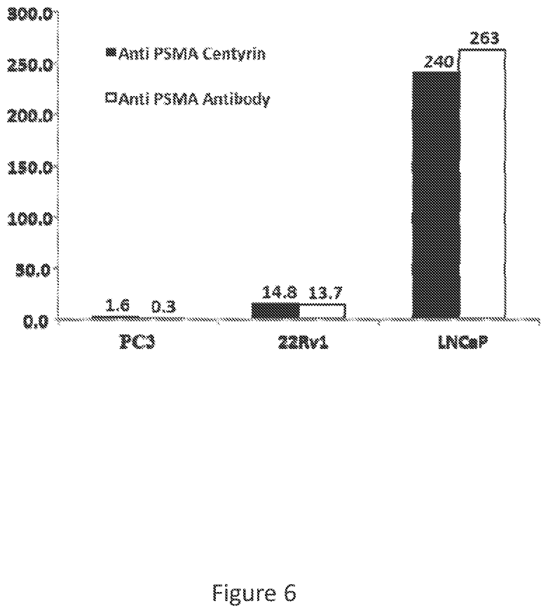

Prostate cell lines were harvested using standard cell culture procedures. The cells (˜30,000) were stained in 0.1 ml of PBS containing 1% bovine serum albumin (BSA) with PE-conjugate Centyrin for 20 minutes. Anti PSMA antibody-PE conjugate from Biolegend (clone LNI-17 catalog #342504) was used as a positive control. After the incubation, 3 ml of PBS/BSA buffer was added and unbound PE conjugate was removed by centrifugation at 800 g for 5 minutes. The supernatant was aspirated and the cells were resuspended in 0.3 ml of PBS/BSA. The samples were analyzed by BD Biosciences FACSCalibur. The mean fluorescent intensity (MFI) of PSMA staining from each cell line was determined and compared to MFI with anti PSMA antibody. The MFI is directly related to PSMA expression level with higher MFI from high PSMA expressing cell line. FIG. 6 shows the MFI values from different cell lines detected with PE-conjugated Centyrin-in comparison to MFI values with anti PSMA antibody-PE.

The results show that a PE-conjugated Centyrin binds to PSMA positive cell lines and does not bind nonspecifically to PSMA negative cells. The MFI is higher with high PSMA expressing cell line (LNCaP) compared to low MFI with low PSMA expressing cell line (22Rv1) as expected. The MFI with PSMA negative cell line (PC3) is close to the background signal. In addition, the performance of PE-conjugated Centyrin E in binding to different cell lines is similar to anti-PSMA antibody-PE, as similar MFI values were obtained with both centyrin and antibody conjugates. This example shows that PE-conjugated Centyrin shows sensitivity and specificity in the detection of PSMA on tumor cells.

### Detection of PSMA by Circulating Tumor Cell Assay

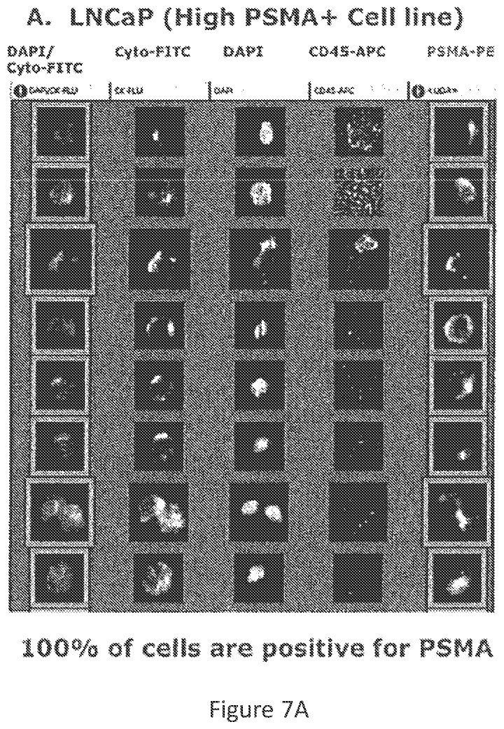

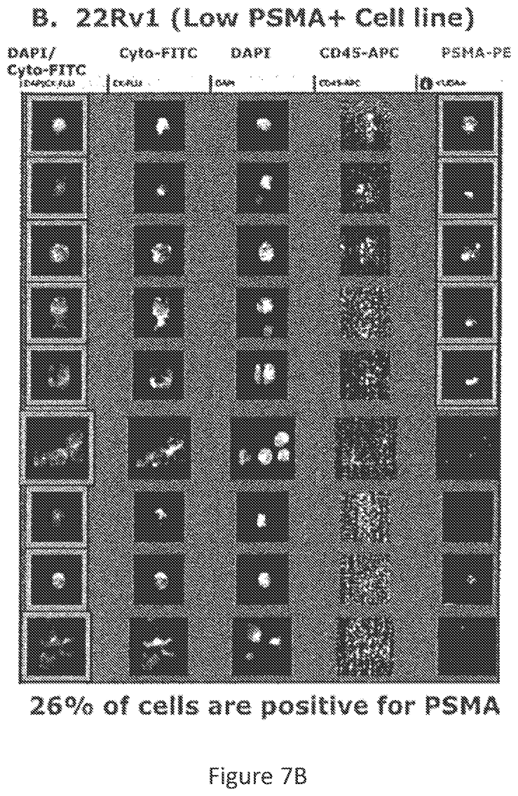

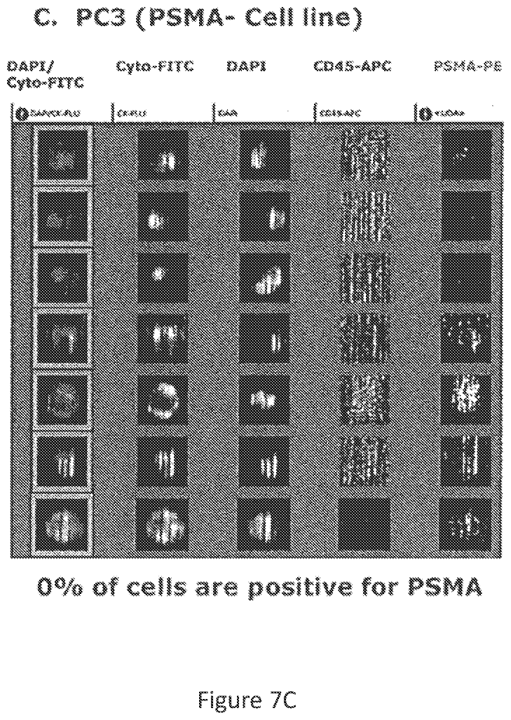

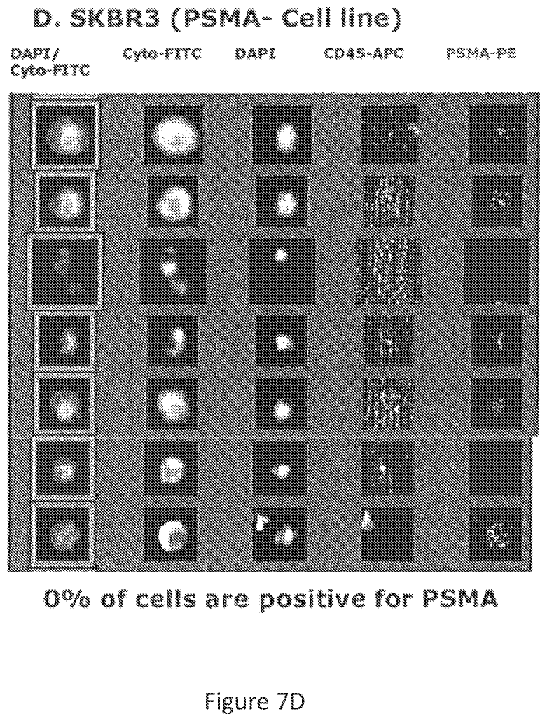

The above results were further confirmed by testing PE-conjugated Centyrin a CELLSEARCH assay to detect and enumerate circulating tumor cells (CTCs) from 7.5 ml of blood. Circulating tumour cell enumeration using the CELLSEARCH (Veridex LLC, Raritan, N.J., USA) was carried out according to the manufacturer's protocol and training. The CELLSEARCH assay uses anti-EpCAM conjugated to ferrofluid magnetic particles to capture and anti-cytokeratin specific to cytokeratins 8, 18 and 19 conjugated to fluorescein to visualize CTCs. The CELLSEARCH assay uses CELLSEARCH AutoPrep for sample preparation and CELLTRACKS Analyzer II® (CTA II) for the analysis. The CTA II is a four color semi-automated fluorescent microscope and uses 3 colors to identify and enumerate CTCs. The fourth color on CTA II is available to phenotype CTCs with additional markers of interest. In this example, tissue cultured tumor cells were spiked into normal blood to mimic CTCs in blood. Approximately 500 tumor cells (LNCaP, 22Rv1, PC3-9 or SKBR3 cells) were spiked into 7.5 ml of normal donor blood collected in a CELLSAVE tube (Janssen Diagnostics). The breast cancer cell line (SKBR3) was also used as PSMA negative cell line. The samples were processed on the AutoPrep using CELLSEARCH CXC kit and PE-conjugated Centyrin as a marker. The AutoPrep sample preparation system enriches tumor cells by capturing tumor cells using anti EpCAM ferrofluid. The CTC enriched samples were stained with a nucleic acid dye (DAPI) to identify nucleated cells, anti-cytokeratin antibody conjugated to fluorescein isothiocyanate (FITC) to identify tumor cells, and anti-leukocyte antibody conjugated to allophycocyanin (APC) to identify leukocytes. The sample was processed to a final volume of 0.32 ml and was transferred to a sample chamber while inside the MagNest® cell presentation device. The MagNest® device presents the magnetically labeled cells for analysis by the CELLTRACKS Analyzer II®. The samples were analyzed using CTAII to enumerate CTCs and detect PSMA on CTCs. The analyzer automatically analyzes samples and presents candidate tumor cells which are positive for DAPI and cytokeratin as thumbnail images for the review. The results from tumor cells stained with PE-conjugated Centyrin in CellSearch assay are shown in FIG. 7. The images CTCs stained with the PE-conjugated Centyrin are in the columns labeled PSMA-PE.

FIG. 7A shows the expression of PSMA on LNCaP tumor cells and 100% of these cells are positive for PSMA. Low PSMA expressing cell line (22Rv1) is 26% positive for PSMA (FIG. 7B). On the other hand, PSMA negative cell lines (PC3-9 and SKBR3) are negative for PSMA (FIGS. 7C and 7D). These results are consistent with flow cytometry results. This example shows that anti PSMA centyrin can be used to detect PSMA expression on CTCs and further confirms the sensitivity and specificity of anti PSMA centyrin.

### Example 11

Tables 14-16 provide a summary of properties for PSMA binding Centyrins H10, D02, and H10 v18. PSMA binding Centyrins were characterized for thermal stability, binding to cynoPSMA-Fc or LnCAP cells as described in Examples 3-5. These illustrate the effectiveness in inhibiting the growth of prostate tumor cells.

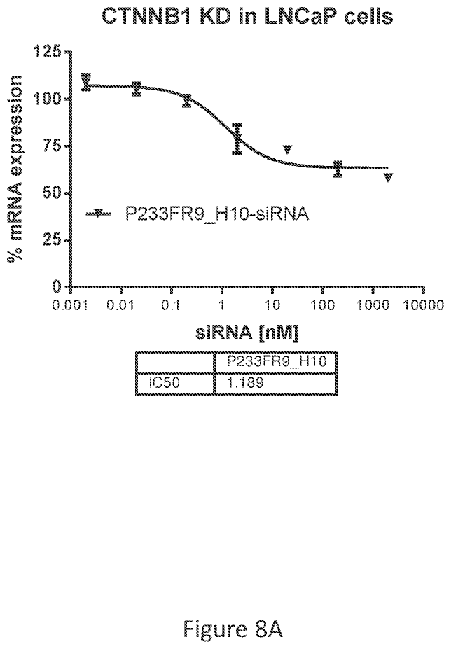

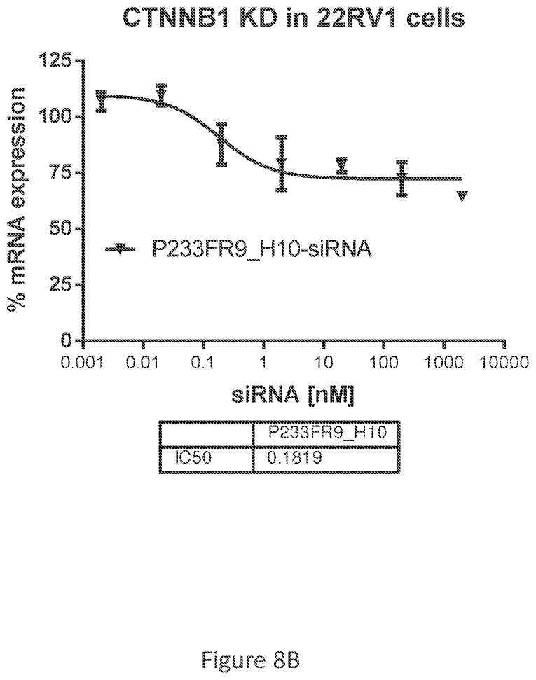

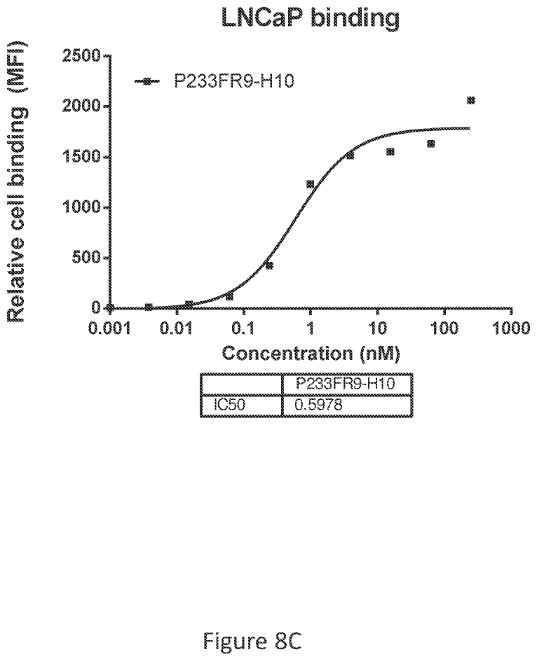

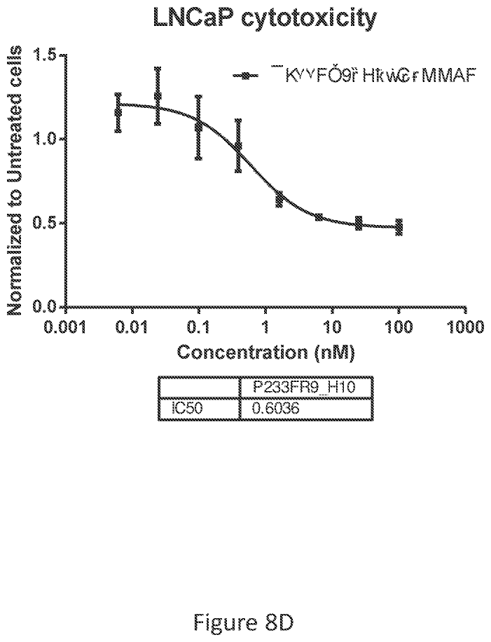

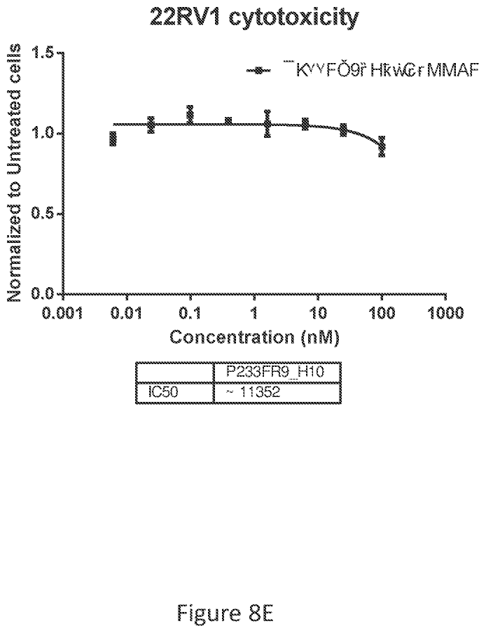

### P233FR9-H10 (SEQ ID NO: 41)—Table 14, FIGS. 8A-8E

TABLE 14

| | P233FR9_H10 | |
|---|---|---|
| Tm | 65.5° | C. |
| % reversible | | |
| Kd (cyno PSMA Fc) | 692 | nM |
| Kd (LNCaP) | ~3 | pM |

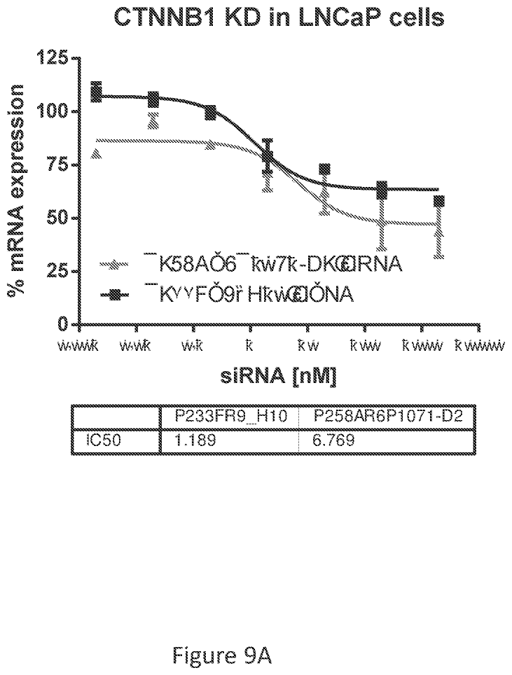

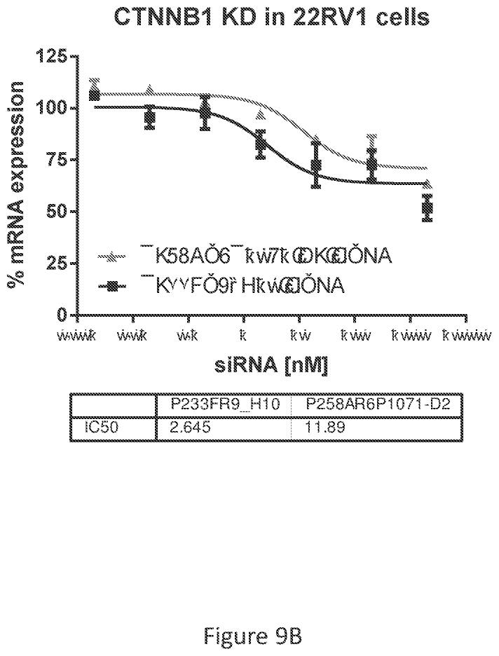

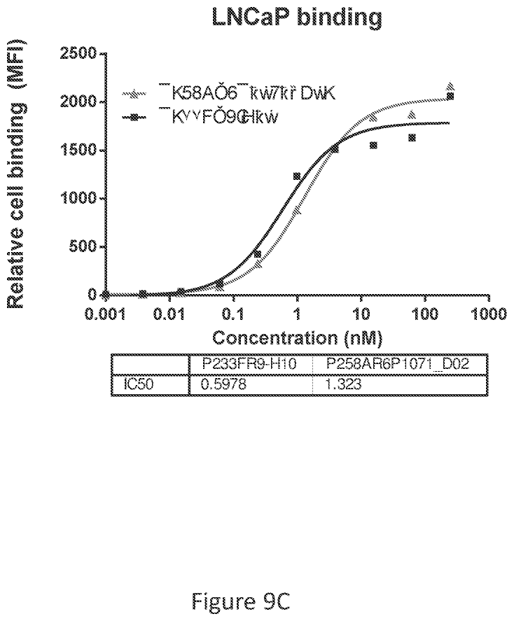

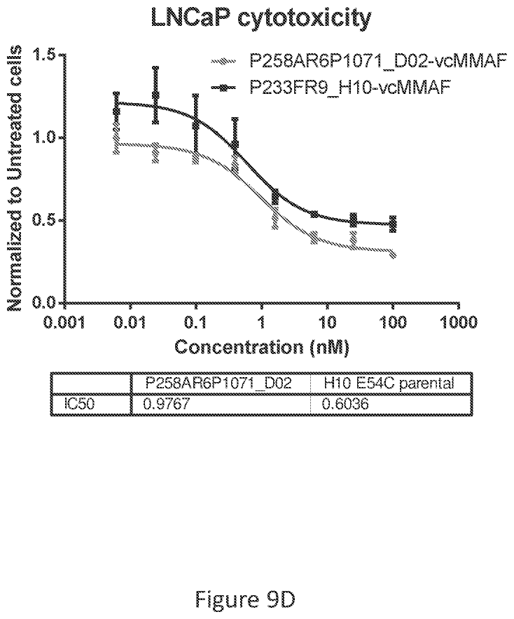

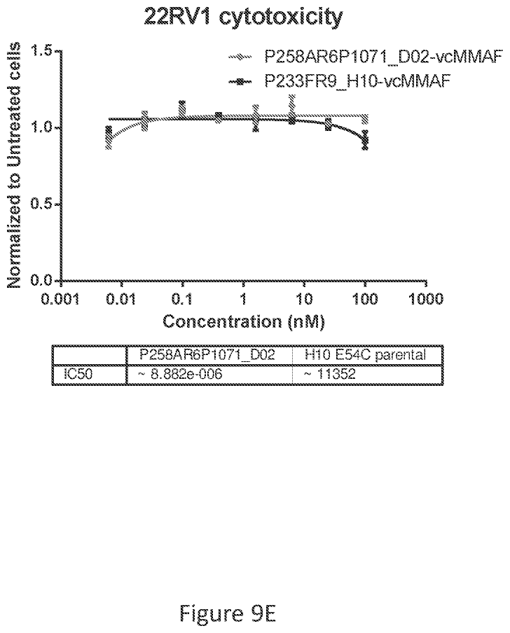

### P258AR6P1071_D02 (SEQ ID NO: 39)—Table 15, FIGS. 9A-9E

TABLE 15

| | P258AR6P1071_D02 | |
|---|---|---|
| Tm | 78.5 | °C |
| % reversible | 45% | |
| Kd (cyno PSMA Fc) | 0.1 | nM |
| Kd (LNCaP) | ~25 | pM |

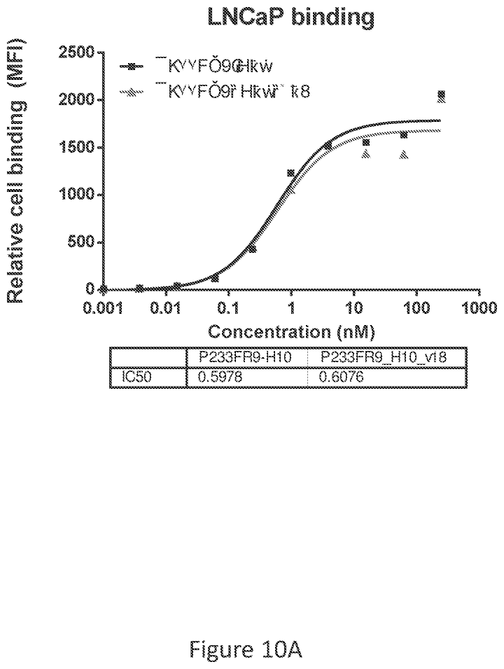

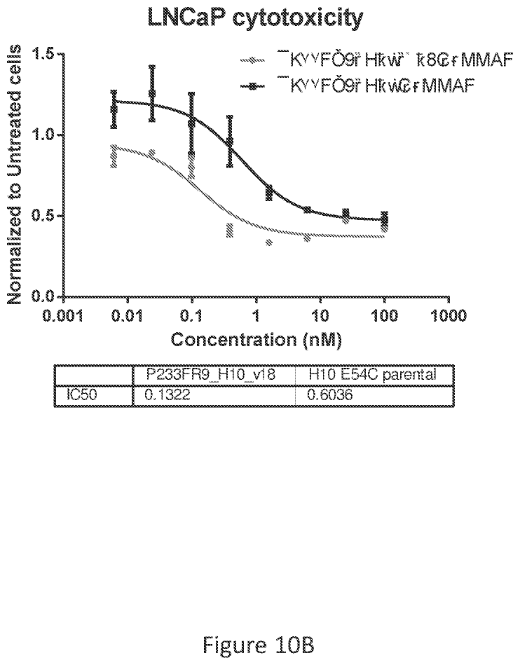

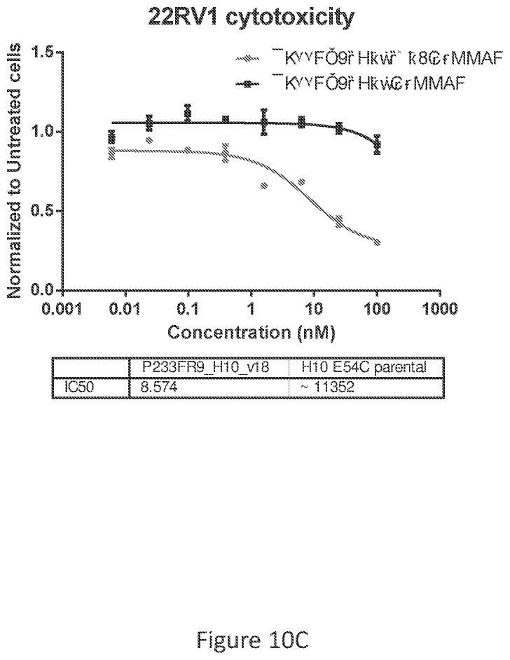

P233FR9_H10_v18 (SEQ ID NO: 111, optimized Centyrin) has improved stability and potency—Table 16, FIGS. 10A-10C

TABLE 16

| | P233FR9_H10_v18 |
|---|---|
| Tm | 71.8° C. |
| % reversible | 56.6% |

### PSMA Centyrin Toxin Conjugates Potently Inhibit PC Xenografts

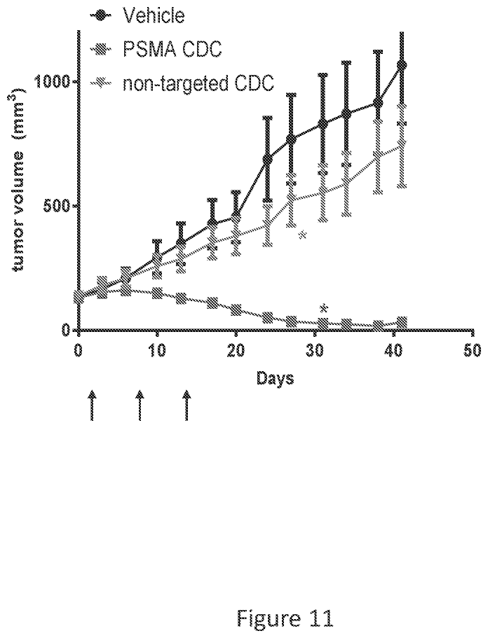

PSMA binding (H10) or negative control (Tencon) Centyrins were expressed and purified as fusions to an albumin binding domain and conjugated to MMAF via a unique cysteine residue introduced to each Centyrin. 3 days before inoculation of SCID-Beige mice with LnCAP tumor cells, pellets containing 5α-dihydrotestostereon were implanted in to the lateral side of the neck between the ear and shoulder via trocar. During implantation, 10×10⁶ viable cells in 0.2 ml matrigel were injected subcutaneously into the upper right flank of each mouse. Dosing of Centyrin-toxin conjugates was initiated when tumor volumes reached approximately 135 mm³. Mice we dosed weekly for three weeks at 10 mg/kg based on mouse weight. Tumor volumes were assessed daily and plotted as a function of time following initiation of dosing (FIG. 11).

The examples and embodiments provided herein demonstrate that PSMA can be effectively used to target cells to PSMA expressing cells to inhibit their growth and, therefore, can be used to treat PSMA expressing tumor cells, such as prostate cancer cells.

### Example 11: Treatment of Prostate Cancer with a Macrophage Comprising a Chimeric Antigen Receptor with a FN3 PSMA Binding Domain

A macrophage is prepared comprising a chimeric antigen receptor (CAR), wherein the CAR comprises at least one heterologous fibronectin type III (FN3) domain as provided for herein, wherein the at least one heterologous FN3 domain is on the surface of the cell and binds to human prostate specific membrane antigen (PSMA), and is operably connected to a transmembrane domain and an intracellular domain of a stimulatory and/or co-stimulatory molecule. The at least one FN3 domain can have the formula of A1-L-A2 as provided for herein. A pharmaceutical composition comprising a population of the macrophages is prepared according to US 2020/0247870 and is administered to a patient with prostate cancer. The prostate cancer is treated with the pharmaceutical composition.

### Sequences as Provided for Herein:

SEQ ID No. 1 = Original Tencon Sequence
LPAPKNLVVSEVTEDSLRLSWTAPDAAFDSFLIQYQESEKVGEAINLTVPGSERS
YDLTGLKPGTEYTVSIYGVKGGHRSNPLSAEFTT
SEQ ID No. 2 = TCL1 library
LPAPKNLVVSEVTEDSLRLSWTAPDAAFDSFLIQYQESEKVGEAINLTVPGSERS
YDLTGLKPGTEYTVSIYGV(X)₇₋₁₂PLSAEFTT;
wherein
X₁, X₂, X₃, X₄, X₅, X₆, X₇ is any amino acid; and
X₈, X₉, X₁₀, X₁₁ and X₁₂ are any amino acid or deleted
SEQ ID No. 3 = TCL2 library
LPAPKNLVVSEVTEDSLRLSWX₁X₂X₃X₄X₅X₆X₇X₈SFLIQYQESEKVGEAINLTVP
GSERSYDLTGLKPGTEYTVSIYGVX₉X₁₀X₁₁X₁₂X₁₃SX₁₄X₁₅LSAEFTT;
wherein
X₁ is Ala, Arg, Asn, Asp, Glu, Gln, Gly, His, Ile, Leu, Lys,
Phe, Pro, Ser, Thr, Trp, Tyr or Val;
X₂ is Ala, Arg, Asn, Asp, Glu, Gln, Gly, His, Ile, Leu, Lys,
Phe, Pro, Ser, Thr, Trp, Tyr or Val;
X₃ Ala, Arg, Asn, Asp, Glu, Gln, Gly, His, Ile, Leu, Lys,
Phe, Pro, Ser, Thr, Trp, Tyr or Val;
X₄ is Ala, Arg, Asn, Asp, Glu, Gln, Gly, His, Ile, Leu, Lys,
Phe, Pro, Ser, Thr, Trp, Tyr or Val;
X₅ is Ala, Arg, Asn, Asp, Glu, Gln, Gly, His, Ile, Leu, Lys,
Phe, Pro, Ser, Thr, Trp, Tyr or Val;
X₆ is Ala, Arg, Asn, Asp, Glu, Gln, Gly, His, Ile, Leu, Lys,
Phe, Pro, Ser, Thr, Trp, Tyr or Val;
X₇ is Phe, Ile, Leu, Val or Tyr;
X₈ is Asp, Glu or Thr;
X₉ is Ala, Arg, Asn, Asp, Glu, Gln, Gly, His, Ile, Leu, Lys,
Phe, Pro, Ser, Thr, Trp, Tyr or Val;
X₁₀ is Ala, Arg, Asn, Asp, Glu, Gln, Gly, His, Ile, Leu, Lys,
Phe, Pro, Ser, Thr, Trp, Tyr or Val;
X₁₁ is Ala, Arg, Asn, Asp, Glu, Gln, Gly, His, Ile, Leu, Lys,
Phe, Pro, Ser, Thr, Trp, Tyr or Val;
X₁₂ is Ala, Arg, Asn, Asp, Glu, Gln, Gly, His, Ile, Leu, Lys,
Phe, Pro, Ser, Thr, Trp, Tyr or Val;
X₁₃ is Ala, Arg, Asn, Asp, Glu, Gln, Gly, His, Ile, Leu, Lys,
Phe, Pro, Ser, Thr, Trp, Tyr or Val;
X₁₄ is Ala, Arg, Asn, Asp, Glu, Gln, Gly, His, Ile, Leu, Lys,
Phe, Pro, Ser, Thr, Trp, Tyr or Val; and
X₁₅ is Ala, Arg, Asn, Asp, Glu, Gln, Gly, His, Ile, Leu, Lys,
Phe, Pro, Ser, Thr, Trp, Tyr or Val.
SEQ ID No. 4 = Stabilized Tencon
LPAPKNLVVSRVTEDSARLSWTAPDAAFDSFLIQYQESEKVGEAIVLTVPGSERS
YDLTGLKPGTEYTVSIYGVKGGHRSNPLSAIFTT
SEQ ID No. 5 = TCL7 (FG and BC loops)
LPAPKNLVVSRVTEDSARLSWX₁X₂X₃X₄X₅X₆X₇X₈X₉FDSFLIQYQESEKVGEAIV
LTVPGSERSYDLTGLKPGTEYTVSIYGVX₁₀X₁₁X₁₂X₁₃X₁₄X₁₅X₁₆X₁₇X₁₈X₁₉SNPLSA
IFTT;
wherein
X₁, X₂, X₃, X₄, X₅, X₆, X₁₀, X₁₁, X₁₂, X₁₃, X₁₄, X₁₅ and X₁₆ are
A, D, E, F, G, H, I, K, L, N, P, Q, R, S, T, V, W or Y; and
X₇, X₈, X₉, X₁₇, X₁₈ and X₁₉, are A, D, E, F, G, H, I, K, L,
N, P, Q, R, S, T, V, W, Y or deleted
SEQ ID No. 6 = TCL9 (FG loop)
LPAPKNLVVSRVTEDSARLSWTAPDAAFDSFLIQYQESEKVGEAIVLTVPGSERS
YDLTGLKPGTEYTVSIYGVX₁X₂X₃X₄X₅X₆X₇X₈X₉X₁₀X₁₁X₁₂SNPLSAIFTT;
wherein
X₁, X₂, X₃, X₄, X₅, X₆ and X₇, is A, D, E, F, G, H, I, K, L,
N, P, Q, R, S, T, V, W or Y; and
X₈, X₉, X₁₀, X₁₁ and X₁₂ is A, D, E, F, G, H, I, K, L, N, P,
Q, R, S, T, V, W, Y or deleted.
SEQ ID NO: 7 = TCL14 library
LPAPKNLVVSRVTEDSARLSWTAPDAAFDSFX₁IX₂YX₃EX₄X₅X₆X₇GEAIVLTVPG
SERSYDLTGLKPGTEYX₈VX₉IX₁₀GVKGGX₁₁X₁₂SX₁₃PLSAIFTT;
wherein
X₁, X₂, X₃, X₄, X₅, X₆, X₇, X₈, X₉, X₁₀, X₁₁, X₁₂ and X₁₃ are A,
D, E, F, G, H, I, K, L, N, P, Q, R, S, T, V, W, Y, C or M.
SEQ ID NO: 8 = TCL24 Library
LPAPKNLVVSRVTEDSARLSWTAPDAAFDSFX₁IX₂YX₃EX₄X₅X₆X₇GEAIX₈LX₉VP
GSERSYDLTGLKPGTEYX₁₀VX₁₁IX₁₂GVKGGX₁₃X₁₄SX₁₅PLX₁₆AX₁₇FTT;
wherein
X₁, X₂, X₃, X₄, X₅, X₆, X₁₀, X₁₁, X₁₂, X₁₃, X₁₄, X₁₅, X₁₆ and X₁₇
are A, D, E, F, G, H, I, K, L, N, P, Q, R, S, T, V, Y or W.
SEQ ID No. 9 = Sloning-FOR
GTGACACGGCGGTTAGAAC
SEQ ID No. 10 = Sloning-REV
GCCTTTGGGAAGCTTCTAAG
SEQ ID No. 11 = POP2250
CGGCGGTTAGAACGCGGCTACAATTAATAC
SEQ ID No. 12 = DigLigRev
CATGATTACGCCAAGCTCAGAA
SEQ ID No. 13 = BC9
GTGACACGGCGGTTAGAACGCGGCTACAATTAATACATAACCCCATCCCCCT
GTTGACAATTAATCATCGGCTCGTATAATGTGTGGAATTGTGAGCGGATAAC
AATTTCACACAGGAAACAGGATCTACCATGCTGCCGGCGCCGAAAAACCTG
GTTGTTTCTGAAGTTACCGAAGACTCTCTGCGTCTGTCTTGGNNNNNNNNNN
NNNNNNNNNNNNNNNNNTTYGACTCTTTCCTGATCCAGTACCAGGAATCTG
AAAAAGTTGGTGAAGCGATCAACCTGACCGTTCCGGGTTCTGAACGTTCTTA
CGACCTGACCGGTCTGAAACCGGGTACCGAATACACCGTTTCTATCTACGGT
GTTCTTAGAAGCTTCCCAAAGGC;
wherein N is any nucleotide.
SEQ ID No. 14 = BC8
GTGACACGGCGGTTAGAACGCGGCTACAATTAATACATAACCCCATCCCCCT
GTTGACAATTAATCATCGGCTCGTATAATGTGTGGAATTGTGAGCGGATAAC
AATTTCACACAGGAAACAGGATCTACCATGCTGCCGGCGCCGAAAAACCTG
GTTGTTTCTGAAGTTACCGAAGACTCTCTGCGTCTGTCTTGGNNNNNNNNNN
NNNNNNNNNNNNNNTTYGACTCTTTCCTGATCCAGTACCAGGAATCTGAAA
AAGTTGGTGAAGCGATCAACCTGACCGTTCCGGGTTCTGAACGTTCTTACGA
CCTGACCGGTCTGAAACCGGGTACCGAATACACCGTTTCTATCTACGGTGTT
CTTAGAAGCTTCCCAAAGGC;
wherein N is any nucleotide.
SEQ ID No. 15 = BC7
GTGACACGGCGGTTAGAACGCGGCTACAATTAATACATAACCCCATCCCCCT
GTTGACAATTAATCATCGGCTCGTATAATGTGTGGAATTGTGAGCGGATAAC
AATTTCACACAGGAAACAGGATCTACCATGCTGCCGGCGCCGAAAAACCTG
GTTGTTTCTGAAGTTACCGAAGACTCTCTGCGTCTGTCTTGGNNNNNNNNNN
NNNNNNNNNNNTTYGACTCTTTCCTGATCCAGTACCAGGAATCTGAAAAAGT
TGGTGAAGCGATCAACCTGACCGTTCCGGGTTCTGAACGTTCTTACGACCTG
ACCGGTCTGAAACCGGGTACCGAATACACCGTTTCTATCTACGGTGTTCTTA
GAAGCTTCCCAAAGGC;
wherein N is any nucleotide.
SEQ ID No. 16 = BC6
GTGACACGGCGGTTAGAACGCGGCTACAATTAATACATAACCCCATCCCCCT
GTTGACAATTAATCATCGGCTCGTATAATGTGTGGAATTGTGAGCGGATAAC
AATTTCACACAGGAAACAGGATCTACCATGCTGCCGGCGCCGAAAAACCTG
GTTGTTTCTGAAGTTACCGAAGACTCTCTGCGTCTGTCTTGGNNNNNNNNNN
NNNNNNNNTTYGACTCTTTCCTGATCCAGTACCAGGAATCTGAAAAAGTTGG
TGAAGCGATCAACCTGACCGTTCCGGGTTCTGAACGTTCTTACGACCTGACC
GGTCTGAAACCGGGTACCGAATACACCGTTTCTATCTACGGTGTTCTTAGAA
GCTTCCCAAAGGC;
wherein N is any nucleotide.
SEQ ID No. 17 = 130 mer-L17A
CGGCGGTTAGAACGCGGCTACAATTAATACATAACCCCATCCCCCTGTTGAC
AATTAATCATCGGCTCGTATAATGTGTGGAATTGTGAGCGGATAACAATTTC
ACACAGGAAACAGGATCTACCATGCTG
SEQ ID No. 18 = POP222ext
CGG CGG TTA GAA CGC GGC TAC AAT TAA TAC
SEQ ID No. 19 = LS1114
CCA AGA CAG ACG GGC AGA GTC TTC GGT AAC GCG AGA AAC AAC CAG
GTT TTT CGG CGC CGG CAG CAT GGT AGA TCC TGT TTC
SEQ ID No. 20 = LS1115
CCG AAG ACT CTG CCC GTC TGT CTT GG
SEQ ID No. 21 = LS1117
CAG TGG TCT CAC GGA TTC CTG GTA CTG GAT CAG GAA AGA GTC GAA
SEQ ID No. 22 = SDG10
CATGCGGTCTCTTCCGAAAAAGTTGGTGAAGCGATCGTCCTGACCGTTCCGG
GT
SEQ ID No. 23 = SDG24
GGTGGTGAAGATCGCAGACAGCGGGTTAG
SEQ ID No. 24 = POP2222
CGGCGGTTAGAACGCGGCTAC
SEQ ID No. 25 = SDG28
AAGATCAGTTGCGGCCGCTAGACTAGAACCGCTGCCACCGCCGGTGGTGAA
GATCGCAGAC
SEQ ID No. 26 = FG12
GTGACACGGCGGTTAGAACGCGGCTACAATTAATACATAACCCCATCCCCCT
GTTGACAATTAATCATCGGCTCGTATAATGTGTGGAATTGTGAGCGGATAAC
AATTTCACACAGGAAACAGGATCTACCATGCTGCCGGCGCCGAAAAACCTG
GTTGTTTCTCGCGTTACCGAAGACTCTGCGCGTCTGTCTTGGACCGCGCCGGA
CGCGGCGTTCGACTCTTTCCTGATCCAGTACCAGGAATCTGAAAAAGTTGGT
GAAGCGATCGTGCTGACCGTTCCGGGTTCTGAACGTTCTTACGACCTGACCG
GTCTGAAACCGGGTACCGAATACACCGTTTCTATCTACGGTGTTNNNNNNNN
NNNNNNNNNNNNNNNNNNNNNNNNNNNNTCTAACCCGCTGTCTGCGATCTT
CACCACCGGCGGTCACCATCACCATCACCATGGCAGCGGTTCTAGTCTAGCG
GCCGCAACTGATCTTGGC;
wherein N is any nucleotide.
SEQ ID No. 27 = FG11
GTGACACGGCGGTTAGAACGCGGCTACAATTAATACATAACCCCATCCCCCT
GTTGACAATTAATCATCGGCTCGTATAATGTGTGGAATTGTGAGCGGATAAC
AATTTCACACAGGAAACAGGATCTACCATGCTGCCGGCGCCGAAAAACCTG
GTTGTTTCTCGCGTTACCGAAGACTCTGCGCGTCTGTCTTGGACCGCGCCGGA
CGCGGCGTTCGACTCTTTCCTGATCCAGTACCAGGAATCTGAAAAAGTTGGT
GAAGCGATCGTGCTGACCGTTCCGGGTTCTGAACGTTCTTACGACCTGACCG
GTCTGAAACCGGGTACCGAATACACCGTTTCTATCTACGGTGTTNNNNNNNN
NNNNNNNNNNNNNNNNNNNNNNNNNTCTAACCCGCTGTCTGCGATCTTCAC
CACCGGCGGTCACCATCACCATCACCATGGCAGCGGTTCTAGTCTAGCGGCC
GCAACTGATCTTGGC;
wherein N is any nucleotide.
SEQ ID No. 28 = FG10
GTGACACGGCGGTTAGAACGCGGCTACAATTAATACATAACCCCATCCCCCT
GTTGACAATTAATCATCGGCTCGTATAATGTGTGGAATTGTGAGCGGATAAC
AATTTCACACAGGAAACAGGATCTACCATGCTGCCGGCGCCGAAAAACCTG
GTTGTTTCTCGCGTTACCGAAGACTCTGCGCGTCTGTCTTGGACCGCGCCGGA
CGCGGCGTTCGACTCTTTCCTGATCCAGTACCAGGAATCTGAAAAAGTTGGT
GAAGCGATCGTGCTGACCGTTCCGGGTTCTGAACGTTCTTACGACCTGACCG
GTCTGAAACCGGGTACCGAATACACCGTTTCTATCTACGGTGTTNNNNNNNN
NNNNNNNNNNNNNNNNNNNNNNTCTAACCCGCTGTCTGCGATCTTCACCAC
CGGCGGTCACCATCACCATCACCATGGCAGCGGTTCTAGTCTAGCGGCCGCA
ACTGATCTTGGC;
wherein N is any nucleotide.
SEQ ID No. 29 = FG9
GTGACACGGCGGTTAGAACGCGGCTACAATTAATACATAACCCCATCCCCCT
GTTGACAATTAATCATCGGCTCGTATAATGTGTGGAATTGTGAGCGGATAAC
AATTTCACACAGGAAACAGGATCTACCATGCTGCCGGCGCCGAAAAACCTG
GTTGTTTCTCGCGTTACCGAAGACTCTGCGCGTCTGTCTTGGACCGCGCCGGA
CGCGGCGTTCGACTCTTTCCTGATCCAGTACCAGGAATCTGAAAAAGTTGGT
GAAGCGATCGTGCTGACCGTTCCGGGTTCTGAACGTTCTTACGACCTGACCG
GTCTGAAACCGGGTACCGAATACACCGTTTCTATCTACGGTGTTNNNNNNNN
NNNNNNNNNNNNNNNNNNNTCTAACCCGCTGTCTGCGATCTTCACCACCGG
CGGTCACCATCACCATCACCATGGCAGCGGTTCTAGTCTAGCGGCCGCAACT
GATCTTGGC;
wherein N is any nucleotide.
SEQ ID No. 30 = FG8
GTGACACGGCGGTTAGAACGCGGCTACAATTAATACATAACCCCATCCCCCT
GTTGACAATTAATCATCGGCTCGTATAATGTGTGGAATTGTGAGCGGATAAC
AATTTCACACAGGAAACAGGATCTACCATGCTGCCGGCGCCGAAAAACCTG
GTTGTTTCTCGCGTTACCGAAGACTCTGCGCGTCTGTCTTGGACCGCGCCGGA
CGCGGCGTTCGACTCTTTCCTGATCCAGTACCAGGAATCTGAAAAAGTTGGT
GAAGCGATCGTGCTGACCGTTCCGGGTTCTGAACGTTCTTACGACCTGACCG
GTCTGAAACCGGGTACCGAATACACCGTTTCTATCTACGGTGTTNNNNNNNN
NNNNNNNNNNNNNNNNTCTAACCCGCTGTCTGCGATCTTCACCACCGGCGGT
CACCATCACCATCACCATGGCAGCGGTTCTAGTCTAGCGGCCGCAACTGATC
TTGGC;
wherein N is any nucleotide.
SEQ ID No. 31 = FG7
GTGACACGGCGGTTAGAACGCGGCTACAATTAATACATAACCCCATCCCCCT
GTTGACAATTAATCATCGGCTCGTATAATGTGTGGAATTGTGAGCGGATAAC
AATTTCACACAGGAAACAGGATCTACCATGCTGCCGGCGCCGAAAAACCTG
GTTGTTTCTCGCGTTACCGAAGACTCTGCGCGTCTGTCTTGGACCGCGCCGGA
CGCGGCGTTCGACTCTTTCCTGATCCAGTACCAGGAATCTGAAAAAGTTGGT
GAAGCGATCGTGCTGACCGTTCCGGGTTCTGAACGTTCTTACGACCTGACCG
GTCTGAAACCGGGTACCGAATACACCGTTTCTATCTACGGTGTTNNNNNNNN
NNNNNNNNNNNNNTCTAACCCGCTGTCTGCGATCTTCACCACCGGCGGTCAC
CATCACCATCACCATGGCAGCGGTTCTAGTCTAGCGGCCGCAACTGATCTTG
GC;
wherein N is any nucleotide.
SEQ ID No. 32 = PSMW1 (N′-AviTag-HisTag-GS-Cyno PSMA_ECD)
KSSSEATNITPKHNMKAFLDELKAENIKKFLHNFTQIPHLAGTEQNFQLAKQIQS
QWKEFGLDSVELTHYDVLLSYPNKTHPNYISIINEDGNEIFNTSLFEPPPAGYENV
SDIVPPFSAFSPQGMPEGDLVYVNYARTEDFFKLERDMKINCSGKIVIARYGKVF
RGNKVKNAQLAGATGVILYSDPDDYFAPGVKSYPDGWNLPGGGVQRGNILNLN
GAGDPLTPGYPANEYAYRRGMAEAVGLPSIPVHPIGYYDAQKLLEKMGGSASP
DSSWRGSLKVPYNVGPGFTGNFSTQKVKMHIHSTSEVTRIYNVIGTLRGAVEPD
RYVILGGHRDSWVFGGIDPQSGAAVVHEIVRSFGMLKKEGWRPRRTILFASWDA
EEFGLLGSTEWAEENSRLLQERGVAYINADSSIEGNYTLRVDCTPLMYSLVYNL
TKELESPDEGFEGKSLYESWTKKSPSPEFSGMPRISKLGSGNDFEVFFQRLGIASG
RARYTKNWETNKFSSYPLYHSVYETYELVEKFYDPMFKYHLTVAQVRGGMVF
ELANSVVLPFDCRDYAVVLRKYADKIYNISMKHPQEMKTYSVSFDSLFSAVKNF
TEIASKFSERLRDFDKSNPILLRMMNDQLMFLERAFIDPLGLPDRPFYRHVIYAPS
SHNKYAGESFPGIYDALFDIESKVDPSQAWGEVKRQISIATFTVQAAAETLSEVA
SEQ ID No. 33 = PSMW8 (N′-AviTag-HisTag-GS-Chimp PSMA_ECD)
KSSNEATNITPKHNMKAFLDELKAENIKKFLYNFTQIPHLAGTEQNFQLAKQIQS
QWKEFGLDSVELAHYDVLLSYPNKTHPNYISIINEDGNEIFNTSLFEPPPPGYENV
LDIVPPFSAFSPQGMPEGDLVYVNYARTEDFFKLERDMKINCSGKIVIARYGKVF
RGNKVKNAQLAGAKGVILYSDPADYFAPGVKSYPDGWNLPGGGVQRGNILNL
NGAGDPLTPGYPANEYAYRHGIAEAVGLPSIPVHPIGYYDAQKLLEKMGGSAPP
DSSWRGSLKVPYNVGPGFTGNFSTQKVKMHIHSTNEVTRIYNVIGTLRGAVEPD
RYVILGGHRDSWVFGGIDPQSGAAVVHEIVRSFGTLKKEGWRPRRTILFASWDA
EEFGLLGSTEWAEENSRLLQERGVAYINADSSIEGNYTLRVDCTPLMYSLVYNL
TKELKSPDEGFEGKSLYESWTKKSPSPEFSGMPRISKLGSGNDFEVFFQRLGIASG
RARYTKNWETNKFSGYPLYHSVYETYELVEKFYDPMFKYHLTVAQVRGGMVF
ELANSIVLPFDCRDYAVVLRKYADKIYNISMKHPQEMKTYSVSFDSLFSAVKNF
TEIASKFTERLQDFDKSNPILLRMMNDQLMFLERAFIDPLGLPDRPFYRHVIYAPS
SHNKYAGESFPGIYDALFDIESKVDPSKAWGDVKRQISVAAFTVQAAAETLSEV
A
SEQ ID NO: 34: hexahistidine tag
HHHHHH
SEQ ID No. 35 = P258AR6P1071_G03
LPAPKNLVVSRVTEDSARLSWDIDEQRDWFDSFLIQYQESEKVGEAIVLTVPGSE
RSYDLTGLKPGTEYTVSIYGVYHVYRSNPLSAIFTT
SEQ ID No. 36 = P258AR6P1070_A05
LPAPKNLVVSRVTEDSARLSWTIDEQRDWFDSFLIQYQESEKVGEAIVLTVPGSE
RSYDLTGLKPGTEYTVSIYGVYHVYRSNPLSAIFTT
SEQ ID No. 37 = P258AR6P1071_F04
LPAPKNLVVSRVTEDSARLSWVIDEQRDWFDSFLIQYQESEKVGEAIVLTVPGSE
RSYDLTGLKPGTEYTVSIYGVYHVYRSNPLSAIFTT
SEQ ID No. 38 = P258AR6P1070_F09
LPAPKNLVVSRVTEDSARLSWTIDEQRDWFESFLIQYQESEKVGEAIVLTVPGSE
RSYDLTGLKPGTEYTVSIYGVYHVYRSNPLSAIFTT
SEQ ID No. 39 = P258AR6P1071_D02
LPAPKNLVVSRVTEDSARLSWAIDEQRDWFESFLIQYQESEKVGEAIVLTVPGSE
RSYDLTGLKPGTEYTVSIYGVYHVYRSNPLSAIFTT
SEQ ID No. 40 = P229CR5P819_H11
LPAPKNLVVSRVTEDSARLSWDIDEQRDWFDSFLIQYQESEKVGEAIVLTVPGSE
RSYDLTGLKPGTEYTVSIYGVYHVYRSSNPLSAIFTT
SEQ ID No. 41 = P233FR9_H10
LPAPKNLVVSRVTEDSARLSWTAPDAAFDSFAIGYWEWDDDGEAIVLTVPGSER
SYDLTGLKPGTEYPVYIAGVKGGQWSFPLSAIFTT
SEQ ID No. 42 = P233FR9P1001_B5-5
LPAPKNLVVSRVTEDSARLSWTAPDAAFDSFTIGYWEWDDDGEAIVLTVPGSER
SYDLTGLKPGTEYPVYIAGVKGGQWSFPLSAIFTT
SEQ ID No. 43 = P233FR9P1001_H3-1
LPAPKNLVVSRVTEDSARLSWTAPDAAFDSFPIGYWEWDDDGEAIVLTVPGSER
SYDLTGLKPGTEYHVYIAGVKGGQWSFPLSAIFTT
SEQ ID No. 44 = P233FR9P1001_D9
LPAPKNLVVSRVTEDSARLSWTAPDAAFDSFPIGYWEWDDDGEAIVLTVPGSER
SYDLTGLKPGTEYWVYIAGVKGGQWSFPLSAIFTT
SEQ ID No. 45 = P234CR9_A07
LPAPKNLVVSRVTEDSARLSWGEQFTIFDSFLIQYQESEKVGEAIVLTVPGSERSY
DLTGLKPGTEYTVSIYGASGYEWFHAFGSSNPLSAIFTT
SEQ ID No. 46 = P234CR9_H01
LPAPKNLVVSRVTEDSARLSWEWWVIPGDFDSFLIQYQESEKVGEAIVLTVPGSE
RSYDLTGLKPGTEYTVSIYGVVNSGQWNDTSNPLSAIFTT
SEQ ID No. 47 = P233FR9_H10 (cterm cys)
LPAPKNLVVSRVTEDSARLSWTAPDAAFDSFAIGYWEWDDDGEAIVLTVPGSER
SYDLTGLKPGTEYPVYIAGVKGGQWSFPLSAIFTTC
SEQ ID No. 48 = P233FR9_H10 (K62C)
LPAPKNLVVSRVTEDSARLSWTAPDAAFDSFAIGYWEWDDDGEAIVLTVPGSER
SYDLTGLCPGTEYPVYIAGVKGGQWSFPLSAIFTT
SEQ ID No. 49 = P233FR9_H10 (E53C)
LPAPKNLVVSRVTEDSARLSWTAPDAAFDSFAIGYWEWDDDGEAIVLTVPGSCR
SYDLTGLKPGTEYPVYIAGVKGGQWSFPLSAIFTT
SEQ ID No. 50 = P233FR9_H10 (R11C)
LPAPKNLVVSCVTEDSARLSWTAPDAAFDSFAIGYWEWDDDGEAIVLTVPGSER
SYDLTGLKPGTEYPVYIAGVKGGQWSFPLSAIFTT
SEQ ID No. 51 = untargeted Centyrin (K62C)
LPAPKNLVVSEVTEDSARLSWTAPDAAFDSFLIQYQESEKVGEAIVLTVPGSERS
YDLTGLCPGTEYTVSIYGVKGGHRSNPLSAIFTTGGHHHHHH
SEQ ID No. 52 = Sortase A
MSHHHHHHSSGENLYFQSKPHIDNYLHDKDKDEKIEQYDKNVKEQASKDKKQ
QAKPQIPKDKSKVAGYIEIPDADIKEPVYPGPATREQLNRGVSFAEENESLDDQNI
SIAGHTFIDRPNYQFTNLKAAKKGSMVYFKVGNETRKYKMTSIRNVKPTAVEVL
DEQKGKDKQLTLITCDDYNEETGVWETRKIFVATEVK
SEQ ID No. 53 = tagless Sortase A
SKPHIDNYLHDKDKDEKIEQYDKNVKEQASKDKKQQAKPQIPKDKSKVAGYIEI
PDADIKEPVYPGPATREQLNRGVSFAEENESLDDQNISIAGHTFIDRPNYQFTNLK
AAKKGSMVYFKVGNETRKYKMTSIRNVKPTAVEVLDEQKGKDKQLTLITCDDY
NEETGVWETRKIFVATEVK
SEQ ID NO: 54 TEV protease cleavage site
ENLYFQS
SEQ ID NO: 55 FG loop of Tencon
KGGHRSN
SEQ ID NO: 56 BC loop
DIDEQRDW
SEQ iD nO: 57 BC loop
TIDEQRDW
SEQ iD NO: 58 BC loop
VIDEQRDW
SEQ ID NO: 59 BC loop
AIDEQRDW
SEQ ID NO: 60 BC loop
EWWVIPGD
SEQ ID NO: 61 BC loop
GEQFTI
SEQ ID NO: 62 BC loop
TAPDAA
SEQ ID NO: 63 C loop
FDSFLIQYQE
SEQ ID NO: 64 C loop
FESFLIQYQE
SEQ ID NO: 65 C loop
FDSFAIGYWE
SEQ ID NO: 66 C loop
FDSFPIGYWE
SEQ ID NO: 67 C loop
FDSFTIGYWE
SEQ ID NO: 68 CD loop
SEKVGE
SEQ ID NO: 69 CD loop
WDDDGE
SEQ ID NO: 70 F loop
TEYTVSIYGV
SEQ ID NO: 71 F loop
TEYTVSIYG
SEQ ID NO: 72 F loop
TEYPVYIAGV
SEQ ID NO: 73 F loop
TEYWVYIAGV
SEQ ID NO: 74 F loop
TEYHVYIAGV
SEQ ID NOs: 75-140, and 173-177 are above in the tables
SEQ ID NO: 141 full length cynoPSMA
MWNLLHETDSAVATARRPRWLCAGALVLAGGFFLLGFLFGWFIKSSSEATNITP
KHNMKAFLDELKAENIKKFLHNFTQIPHLAGTEQNFQLAKQIQSQWKEFGLDSV
ELTHYDVLLSYPNKTHPNYISIINEDGNEIFNTSLFEPPPAGYENVSDIVPPFSAFSP
QGMPEGDLVYVNYARTEDFFKLERDMKINCSGKIVIARYGKVFRGNKVKNAQL
AGATGVILYSDPDDYFAPGVKSYPDGWNLPGGGVQRGNILNLNGAGDPLTPGY
PANEYAYRRGMAEAVGLPSIPVHPIGYYDAQKLLEKMGGSASPDSSWRGSLKV
PYNVGPGFTGNFSTQKVKMHIHSTSEVTRIYNVIGTLRGAVEPDRYVILGGHRDS
WVFGGIDPQSGAAVVHEIVRSFGMLKKEGWRPRRTILFASWDAEEFGLLGSTEW
AEENSRLLQERGVAYINADSSIEGNYTLRVDCTPLMYSLVYNLTKELESPDEGFE
GKSLYESWTKKSPSPEFSGMPRISKLGSGNDFEVFFQRLGIASGRARYTKNWETN
KFSSYPLYHSVYETYELVEKFYDPMFKYHLTVAQVRGGMVFELANSVVLPFDC
RDYAVVLRKYADKIYNISMKHPQEMKTYSVSFDSLFSAVKNFTEIASKFSERLRD
FDKSNPILLRMMNDQLMFLERAFIDPLGLPDRPFYRHVIYAPSSHNKY
SEQ ID NO: 142 linker
AEAAAKEAAAKEAAAKEAAAKEAAAKAAA
SEQ ID NO: 143 human PSMA ECD
KSSNEATNITPKHNMKAFLDELKAENIKKFLYNFTQIPHLAGTEQNFQLAKQIQS
QWKEFGLDSVELAHYDVLLSYPNKTHPNYISIINEDGNEIFNTSLFEPPPPG
YENVSDIVPPFSAFSPQGMPEGDLVYVNYARTEDFFKLERDMKINCSGKI
VIARYGKVFRGNKVKNAQLAGAKGVILYSDPADYFAPGVKSYPDGWNL
PGGGVQRGNILNLNGAGDPLTPGYPANEYAYRRGIAEAVGLPSIPVHPIG
YYDAQKLLEKMGGSAPPDSSWRGSLKVPYNVGPGFTGNFSTQKVKMHI
HSTNEVTRIYNVIGTLRGAVEPDRYVILGGHRDSWVFGGIDPQSGAAVVH
EIVRSFGTLKKEGWRPRRTILFASWDAEEFGLLGSTEWAEENSRLLQERG
VAYINADSSIEGNYTLRVDCTPLMYSLVHNLTKELKSPDEGFEGKSLYES
WTKKSPSPEFSGMPRISKLGSGNDFEVFFQRLGIASGRARYTKNWETNKF
SGYPLYHSVYETYELVEKFYDPMFKYHLTVAQVRGGMVFELANSIVLPF
DCRDYAVVLRKYADKIYSISMKHPQEMKTYSVSFDSLFSAVKNFTEIASK
FSERLQDFDKSNPIVLRMMNDQLMFLERAFIDPLGLPDRPFYRHVIYAPSS
HNKYAGESFPGIYDALFDIESKVDPSKAWGEVKRQIYVAAFTVQAAAET
LSEVA
SEQ ID NO: 144 human FL PSMA with signal sequence
MWNLLHETDSAVATARRPRWLCAGALVLAGGFFLLGFLFGWFIKSSNEATNITP
KHNMKAFLDELKAENIKKFLYNFTQIPHLAGTEQNFQLAKQIQSQWKEF
GLDSVELAHYDVLLSYPNKTHPNYISIINEDGNEIFNTSLFEPPPPGYENVS
DIVPPFSAFSPQGMPEGDLVYVNYARTEDFFKLERDMKINCSGKIVIARY
GKVFRGNKVKNAQLAGAKGVILYSDPADYFAPGVKSYPDGWNLPGGGV
QRGNILNLNGAGDPLTPGYPANEYAYRRGIAEAVGLPSIPVHPIGYYDAQ
KLLEKMGGSAPPDSSWRGSLKVPYNVGPGFTGNFSTQKVKMHIHSTNEV
TRIYNVIGTLRGAVEPDRYVILGGHRDSWVFGGIDPQSGAAVVHEIVRSF
GTLKKEGWRPRRTILFASWDAEEFGLLGSTEWAEENSRLLQERGVAYIN
ADSSIEGNYTLRVDCTPLMYSLVHNLTKELKSPDEGFEGKSLYESWTKKS
PSPEFSGMPRISKLGSGNDFEVFFQRLGIASGRARYTKNWETNKFSGYPL
YHSVYETYELVEKFYDPMFKYHLTVAQVRGGMVFELANSIVLPFDCRDY
AVVLRKYADKIYSISMKHPQEMKTYSVSFDSLFSAVKNFTEIASKFSERL
QDFDKSNPIVLRMMNDQLMFLERAFIDPLGLPDRPFYRHVIYAPSSHNKY
AGESFPGIYDALFDIESKVDPSKAWGEVKRQIYVAAFTVQAAAETLSEVA
SEQ ID NO: 145 3rd FN3 domain of tenascin C
DAPSQIEVKDVTDTTALITWFKPLAEIDGIELTYGIKDVPGDRTTIDLTEDENQYSI
GNLKPDTEYEVSLISRRGDMSSNPAKETFTT
SEQ ID NO: 146 Fibcon
LDAPTDLQVTNVTDTSITVSWTPPSATITGYRITYTPSNGPGEPKELTVPPSSTSVT
ITGLTPGVEYVVSLYALKDNQESPPLVGTQTT
SEQ ID NO: 147 10^{th} FN3 domain of fibronectin
VSDVPRDLEVVAATPTSLLISWDAPAVTVRYYRITYGETGGNSPVQEFTVPGSKS
TATISGLKPGVDYTITVYAVTGRGDSPASSKPISINYRT
SEQ ID NO: 148 linker
GSGS
SEQ ID NO: 149 (GGGS)₂ linker
GGGSGGGS
SEQ ID NO: 150 (GGGS)₅ linker
GGGGSGGGGSGGGGSGGGGSGGGGS
SEQ ID NO: 151 linker
APAP
SEQ ID NO: 152 (AP)₅ linker
APAPAPAPAP
SEQ ID NO: 153 (AP)₁₀ linker
APAPAPAPAPAPAPAPAPAP
SEQ ID NO: 154 (AP)₂₀ linker
APAPAPAPAPAPAPAPAPAPAPAPAPAPAPAPAPAPAPAP
SEQ ID NO: 155 Albumin variant
DAHKSEVAHRFKDLGEENFKALVLIAFAQYLQQSPFEDHVKLVNEVTEFAKTCV
ADESAENCDKSLHTLFGDKLCTVATLRETYGEMADCCAKQEPERNECFL
QHKDDNPNLPRLVRPEVDVMCTAFHDNEETFLKKYLYEIARRHPYFYAP
ELLFFAKRYKAAFTECCQAADKAACLLPKLDELRDEGKASSAKQRLKCA
SLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRL
AKTYETTLEKCCAAADPHECYAKVFDEFKPLVEEPQNLIKQNCELFEQLG
EYKFQNALLVRYTKKVPQVSTPTLVEVSRNLGKVGSKCCKHPEAKRMPC
AEDYLSVVLNQLCVLHEKTPVSDRVTKCCTESLVNRRPCFSALEVDETY
VPKEFNAETFTFHADICTLSEKERQIKKQTALVELVKHKPKATKEQLKAV
MDDFAAFVEKCCKADDKETCFAEEGKKLVAASQAALGL
SEQ ID NO: 156 cDNA H10
CTGCCAGCCCCGAAGAATTTGGTCGTTTCCCGTGTCACTGAGGACTCTGCAC
GTCTGAGCTGGACCGCACCGGACGCGGCGTTCGACAGCTTTGCAATCGGCTA
CTGGGAGTGGGATGATGACGGCGAGGCCATTGTGCTGACCGTTCCGGGTAGC
GAGCGCAGCTACGATCTGACCGGTCTGAAGCCGGGTACGGAATATCCGGTGT
ATATTGCGGGCGTGAAGGGTGGCCAGTGGAGCTTCCCGCTGAGCGCGATCTT
TACCACC
SEQ ID NO: 157 cDNA P258AR6P1071_D02
CTGCCGGCTCCGAAAAACCTGGTCGTTTCCCGTGTCACTGAAGATTCTGCAC
GCTTGAGCTGGGCGATCGACGAGCAGCGTGACTGGTTTGAGAGCTTCCTGAT
TCAGTATCAAGAATCGGAAAAAGTTGGCGAGGCCATCGTGCTGACCGTTCCG
GGTAGCGAGCGCAGCTATGATCTGACGGGTCTGAAGCCAGGCACCGAGTAT
ACGGTGAGCATTTACGGTGTCTACCATGTGTACCGTAGCAATCCGCTGAGCG
CGATCTTCACCACC
SEQ ID NO: 158 cDNA
P233FR9P1001_H3-1
CTGCCAGCCCCGAAAAACTTAGTTGTCTCCCGCGTGACCGAAGATTCTGCTC
GTCTGAGCTGGACTGCACCGGACGCGGCGTTCGACAGCTTTCCGATTGGCTA
CTGGGAGTGGGATGATGACGGTGAAGCGATCGTGCTGACCGTTCCGGGTAGC
GAGCGTAGCTATGACCTGACGGGTTTGAAACCTGGTACCGAGTATCACGTTT
ACATTGCGGGCGTCAAGGGTGGCCAGTGGTCGTTCCCGCTGAGCGCAATCTT
TACGACC
SEQ ID NO: 159 PSMA epitope
KKSPSPEFSGMPRISK
SEQ ID NO: 160 PSMA epitope
NWETNKF
SEQ ID NO: 161 PSMA Cen17
MLPAPKNLVVSRVTEDSARLSWAIDEQRDWFESFLIQYQESEKVGEAIVLTVPGS
ERSYDLTGLKPGTEYTVSIYGVYHVYRSNPLSAIFTTGGGGSGGGGSGGGGSGG
GGSLPAPKNLVVSRVTEDSARLSWTAPDAAFDSFAIGYWEWDDDGEAIVLTVP
GSERSYDLTGLKPGTEYPVYIAGVKGGQWSFPLSAIFTT
SEQ ID NO: 162 (GGGS)₄ linker
GGGSGGGSGGGSGGGS
SEQ ID NO: 163 (GGGS)₃ linker
GGGSGGGSGGGS
SEQ ID NO: 164 (Signal Sequence)
MALPVTALLLPLALLLHAARP
SEQ ID NO: 165 (CD8 hinge)
TTTPAPRPPTPAPTIASQPLSLRPEACRPAAGGAVHTRGLDFACDIYIWAPLAGTC
GVLLLSLVITLYC
SEQ ID NO: 166 (H10 with signal sequence and CD8 hinge)
MALPVTALLLPLALLLHAARPLPAPKNLVVSRVTEDSARLSWTAPDAAFDSFAI
GYWEWDDDGEAIVLTVPGSERSYDLTGLKPGTEYPVYIAGVKGGQWSFPLSAIF
TTTTTPAPRPPTPAPTIASQPLSLRPEACRPAAGGAVHTRGLDFACDIYIWAPLAG
TCGVLLLSLVITLYC
SEQ ID NO: 167 (D02 with signal sequence and CD8 hinge)
MALPVTALLLPLALLLHAARPLPAPKNLVVSRVTEDSARLSWAIDEQRDWFESF
LIQYQESEKVGEAIVTVPGSERSYDLTGLKPGTEYTVSIYGVYHVYRSNPLSAIFT
TTTTPAPRPPTPAPTIASQPLSLRPEACRPAAGGAVHTRGLDFACDIYIWAPLAGT
CGVLLLSLVITLY
SEQ ID NO: 168 (H10-v31)
LPAPKNLVVSRVTEDSARLSWTAPDAAFDSFAIGYWEWDDDGEAIVLTVPGSER
SYDLTGLKPGTEYPVYIAGVKGGQWSFPLSAIFTT
SEQ ID NO: 169 (H10-v32)
LPAPKNLVVSRVTEDSARLSWTAPDAAFDSFAIGYWEWDDDGEAIVLTVPGSER
SYDLTGLKPGTEYPVYIAGVKGGQWSFPLSAIFTT
SEQ ID NO: 170 (H10-v31 with signal sequence, linker, and CD8 hinge)
MALPVTALLLPLALLLHAARPLPAPKNLVVSRVTEDSARLSWTAPDAAFDSFAI
GYWEWDDDGEAIVLTVPGSERSYDLTGLKPGTEYPVYIAGVKGGQWSFPLSAIF
TTGGGGSGGGGSGGGGSTTTPAPRPPTPAPTIASQPLSLRPEACRPAAGGAVHTR
GLDFACDIYIWAPLAGTCGVLLLSLVITLYC
SEQ ID NO: 171 (H10-v32 with signal sequence, linker, and CD8 hinge)
MALPVTALLLPLALLLHAARPLPAPKNLVVSRVTEDSARLSWTAPDAAFDSFAI
GYWEWDDDGEAIVLTVPGSERSYDLTGLKPGTEYPVYIAGVKGGQWSFPLSAIF
TTGGGGSGGGGSTTTPAPRPPTPAPTIASQPLSLRPEACRPAAGGAVHTRGLDFA
CDIYIWAPLAGTCGVLLLSLVITLYC
SEQ ID NO: 172 (D02-v14 with signal sequence, linker, and CD8 hinge)
MALPVTALLLPLALLLHAARPLPAPKNLVVSRVTEDSARLSWDIDEQRDWFESF
LIQYQESEKVGEAIVTVPGSERSYDLTGLKPGTEYTVSIYGVYHVYRSNPLSAIFT
TTTTPAPRPPTPAPTIASQPLSLRPEACRPAAGGAVHTRGLDFACDIYIWAPLAGT
CGVLLLSLVITLY

1. A macrophage comprising a chimeric antigen receptor comprising at least one heterologous fibronectin type III (FN3) domain,
wherein the at least one heterologous FN3 domain is on the surface of the cell and binds to human prostate specific membrane antigen (PSMA),
wherein the at least one heterologous FN3 domain is operably connected to a transmembrane domain, and wherein the transmembrane domain is operably connected to an intracellular domain of a stimulatory or co-stimulatory molecule, and
wherein the at least one heterologous FN3 domain has an amino acid sequence selected from the group consisting of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, 169, and combinations thereof.

2.-3. (canceled)

4. The macrophage of claim 1, wherein the intracellular domain further comprises a hinge region.

5. The macrophage of claim 4, wherein the hinge domain comprises a CD8 hinge domain or an Ig hinge domain.

6. The macrophage of claim 1, wherein the transmembrane domain is selected from a CD8 transmembrane domain, a CD64 transmembrane domain, a CD16 transmembrane domain, a TLR1 transmembrane domain, a TLR2 transmembrane domain, a TLR4 transmembrane domain, a TLR5 transmembrane domain, or a TLR6 transmembrane domain.

7. The macrophage of claim 1, wherein the intracellular domain comprises dual signaling domains.

8. The macrophage of claim 1, wherein the intracellular domain is selected from a CD3 zeta intracellular domain, an FcεRI commonγ subunit intracellular domain, a Dectin-1 intracellular domain, a CD16 intracellular domain, a TLR1 intracellular domain, a TLR2 intracellular domain, a TLR4 intracellular domain, a TLR5 intracellular domain, or a TLR6 intracellular domain.

9. The macrophage of claim 1, further comprising one or more spacer domains operably connecting the transmembrane domain to the FN3 domain or the transmembrane domain to the intracellular domain.

10. (canceled)

11. The macrophage of claim 1, wherein the at least one heterologous FN3 domain that binds PSMA has an amino acid sequence of SEQ ID NO: 39.

12. The macrophage of claim 1, wherein the at least one heterologous FN3 domain that binds PSMA has an amino acid sequence of SEQ ID NO: 41.

13. The macrophage of claim 1, wherein the chimeric antigen receptor comprises a first heterologous FN3 domain and a second heterologous FN3 domain that bind to PSMA.

14. The macrophage of claim 13, wherein the first heterologous FN3 domain and the second heterologous FN3 domain bind to different epitopes on PSMA.

15. The macrophage of claim 14, wherein the different epitopes on PSMA do not overlap.

16. The macrophage of claim 13, wherein the first heterologous FN3 domain and the second FN3 domain are different.

17. The macrophage of claim 13, wherein the first heterologous FN3 domain and the second heterologous FN3 domain each, independently, have an amino acid sequence selected from the group consisting of SEQ ID NOs: 35, 36, 37, 38, 39, 40, 41, 42, 43, 44, 45, 46, 47, 48, 49, 50, 51, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 168, and 169.

18. The macrophage of claim 13, wherein the chimeric antigen receptor comprises from N-terminus to C-terminus the first heterologous FN3 domain followed by the second heterologous FN3 domain.

19. The macrophage of claim 13, wherein the chimeric antigen receptor comprises from N-terminus to C-terminus the second heterologous FN3 domain followed by the first heterologous FN3 domain.

20. The macrophage of claim 13, wherein the first heterologous FN3 domain having an amino acid sequence of SEQ ID NO: 39 and the second heterologous FN3 domain having an amino acid sequence of SEQ ID NO: 41.

21. The macrophage of claim 13, wherein the first heterologous FN3 domain and second heterologous FN3 domain are connected by a linker.

22.-24. (canceled)

25. The macrophage of claim 21, wherein the linker comprises an amino acid sequence of SEQ ID NOs: 148, 149, 150, 151, 152, 153, 154, 142, 162, or 163.

26. (canceled)

27. The macrophage of claim 1, wherein the chimeric antigen receptor comprises an amino acid sequence of SEQ ID NO: 161.

28. The macrophage of claim 13, wherein the first heterologous FN3 domain and the second heterologous FN3 domain are the same.

29-34. (canceled)

35. A pharmaceutical composition comprising a cell of claim 1.

36-46. (canceled)

47. A method of treating prostate cancer in a subject, the method comprising administering the pharmaceutical composition of claim 35.

48-50. (canceled)


SEQUENCE LISTING (177 sequences)

SEQ ID NO: 1 - protein, 89 aa, Artificial Sequence
  synthetic sequence
LPAPKNLVVSEVTEDSLRLSWTAPDAAFDSFLIQYQESEKVGEAINLTVPGSERSYDLTGLKPGTEYTVSIYGVKGGHRSNPLSAEFTT

SEQ ID NO: 2 - protein, 94 aa, Artificial Sequence
  synthetic sequence
  MISC_FEATURE (75)..(81): each X is any amino acid.
  MISC_FEATURE (82)..(86): each X is any amino acid or deleted.
LPAPKNLVVSEVTEDSLRLSWTAPDAAFDSFLIQYQESEKVGEAINLTVPGSERSYDLTGLKPGTEYTVSIYGVXXXXXXXXXXXXPLSAEFTT

SEQ ID NO: 3 - protein, 89 aa, Artificial Sequence
  synthetic sequence
  MISC_FEATURE (22)..(27): each X is Ala, Arg, Asn, Asp, Glu, Gln, Gly, His, Ile, Leu, Lys, Phe, Pro, Ser, Thr, Trp, Tyr or Val
  MISC_FEATURE (28)..(28): X is Phe, Ile, Leu, Val or Tyr
  MISC_FEATURE (29)..(29): X is Asp, Glu or Thr
  MISC_FEATURE (75)..(79): each X is Ala, Arg, Asn, Asp, Glu, Gln, Gly, His, Ile, Leu, Lys, Phe, Pro, Ser, Thr, Trp, Tyr or Val
  MISC_FEATURE (81)..(82): each X is Ala, Arg, Asn, Asp, Glu, Gln, Gly, His, Ile, Leu, Lys, Phe, Pro, Ser, Thr, Trp, Tyr or Val
LPAPKNLVVSEVTEDSLRLSWXXXXXXXXSFLIQYQESEKVGEAINLTVPGSERSYDLTGLKPGTEYTVSIYGVXXXXXSXXLSAEFTT

SEQ ID NO: 4 - protein, 89 aa, Artificial Sequence
  synthetic sequence
LPAPKNLVVSRVTEDSARLSWTAPDAAFDSFLIQYQESEKVGEAIVLTVPGSERSYDLTGLKPGTEYTVSIYGVKGGHRSNPLSAIFTT

SEQ ID NO: 5 - protein, 97 aa, Artificial Sequence
  synthetic sequence
  MISC_FEATURE (22)..(27): each X is A, D, E, F, G, H, I, K, L, N, P, Q, R, S, T, V, W or Y
  MISC_FEATURE (28)..(30): each X is A, D, E, F, G, H, I, K, L, N, P, Q, R, S, T, V, W, Y or deleted
  MISC_FEATURE (78)..(84): each X is A, D, E, F, G, H, I, K, L, N, P, Q, R, S, T, V, W or Y
  MISC_FEATURE (85)..(87): each X is A, D, E, F, G, H, I, K, L, N, P, Q, R, S, T, V, W, Y or deleted
LPAPKNLVVSRVTEDSARLSWXXXXXXXXXFDSFLIQYQESEKVGEAIVLTVPGSERSYDLTGLKPGTEYTVSIYGVXXXXXXXXXXSNPLSAIFTT

SEQ ID NO: 6 - protein, 96 aa, Artificial Sequence
  synthetic sequence
  misc_feature (75)..(75): Xaa can be any naturally occurring amino acid
  MISC_FEATURE (76)..(82): each X is A, D, E, F, G, H, I, K, L, N, P, Q, R, S, T, V, W or Y
  MISC_FEATURE (83)..(87): each X is A, D, E, F, G, H, I, K, L, N, P, Q, R, S, T, V, W, Y or deleted
LPAPKNLVVSRVTEDSARLSWTAPDAAFDSFLIQYQESEKVGEAIVLTVPGSERSYDLTGLKPGTEYTVSIYGVXXXXXXXXXXXXSNPLSAIFTT

SEQ ID NO: 7 - protein, 89 aa, Artificial Sequence
  synthetic sequence
  MISC_FEATURE (32)..(32): X is A, D, E, F, G, H, I, K, L, N, P, Q, R, S, T, V, W,Y, C or M
  MISC_FEATURE (34)..(34): X is A, D, E, F, G, H, I, K, L, N, P, Q, R, S, T, V, W,Y, C or M
  MISC_FEATURE (36)..(36): X is A, D, E, F, G, H, I, K, L, N, P, Q, R, S, T, V, W,Y, C or M
  MISC_FEATURE (38)..(41): each X is A, D, E, F, G, H, I, K, L, N, P, Q, R, S, T, V, W,Y, C or M
  MISC_FEATURE (68)..(68): X is A, D, E, F, G, H, I, K, L, N, P, Q, R, S, T, V, W,Y, C or M
  MISC_FEATURE (70)..(70): X is A, D, E, F, G, H, I, K, L, N, P, Q, R, S, T, V, W,Y, C or M
  MISC_FEATURE (72)..(72): X is A, D, E, F, G, H, I, K, L, N, P, Q, R, S, T, V, W,Y, C or M
  MISC_FEATURE (78)..(79): each X is A, D, E, F, G, H, I, K, L, N, P, Q, R, S, T, V, W,Y, C or M
  MISC_FEATURE (81)..(81): X is A, D, E, F, G, H, I, K, L, N, P, Q, R, S, T, V, W,Y, C or M
LPAPKNLVVSRVTEDSARLSWTAPDAAFDSFXIXYXEXXXXGEAIVLTVPGSERSYDLTGLKPGTEYXVXIXGVKGGXXSXPLSAIFTT

SEQ ID NO: 8 - protein, 89 aa, Artificial Sequence
  synthetic sequence
  MISC_FEATURE (32)..(32): X is A, D, E, F, G, H, I, K, L, N, P, Q, R, S, T, V, Y or W
  MISC_FEATURE (34)..(34): X is A, D, E, F, G, H, I, K, L, N, P, Q, R, S, T, V, Y or W
  MISC_FEATURE (36)..(36): X is A, D, E, F, G, H, I, K, L, N, P, Q, R, S, T, V, Y or W
  MISC_FEATURE (38)..(41): each X is A, D, E, F, G, H, I, K, L, N, P, Q, R, S, T, V, Y or W
  MISC_FEATURE (46)..(46): X is A, D, E, F, G, H, I, K, L, N, P, Q, R, S, T, V, Y or W
  MISC_FEATURE (48)..(48): X is A, D, E, F, G, H, I, K, L, N, P, Q, R, S, T, V, Y or W
  MISC_FEATURE (68)..(68): X is A, D, E, F, G, H, I, K, L, N, P, Q, R, S, T, V, Y or W
  MISC_FEATURE (70)..(70): X is A, D, E, F, G, H, I, K, L, N, P, Q, R, S, T, V, Y or W
  MISC_FEATURE (72)..(72): X is A, D, E, F, G, H, I, K, L, N, P, Q, R, S, T, V, Y or W
  MISC_FEATURE (78)..(79): each X is A, D, E, F, G, H, I, K, L, N, P, Q, R, S, T, V, Y or W
  MISC_FEATURE (81)..(81): X is A, D, E, F, G, H, I, K, L, N, P, Q, R, S, T, V, Y or W
  MISC_FEATURE (84)..(84): X is A, D, E, F, G, H, I, K, L, N, P, Q, R, S, T, V, Y or W
  MISC_FEATURE (86)..(86): X is A, D, E, F, G, H, I, K, L, N, P, Q, R, S, T, V, Y or W
LPAPKNLVVSRVTEDSARLSWTAPDAAFDSFXIXYXEXXXXGEAIXLXVPGSERSYDLTGLKPGTEYXVXIXGVKGGXXSXPLXAXFTT

SEQ ID NO: 9 - DNA, 19 bp, Artificial Sequence
  synthetic sequence
gtgacacggcggttagaac

SEQ ID NO: 10 - DNA, 20 bp, Artificial Sequence
  synthetic sequence
gcctttgggaagcttctaag

SEQ ID NO: 11 - DNA, 30 bp, Artificial Sequence
  synthetic sequence
cggcggttagaacgcggctacaattaatac

SEQ ID NO: 12 - DNA, 22 bp, Artificial Sequence
  synthetic sequence
catgattacgccaagctcagaa

SEQ ID NO: 13 - DNA, 385 bp, Artificial Sequence
  synthetic sequence
  misc_feature (198)..(224): each n is any nucleotide
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctgaagttaccgaagactctctgcgtctgtcttggnnnnnnnnnnnnnnnnnnnnnnnnnnnttygactctttcctgatccagtaccaggaatctgaaaaagttggtgaagcgatcaacctgaccgttccgggttctgaacgttcttacgacctgaccggtctgaaaccgggtaccgaatacaccgtttctatctacggtgttcttagaagcttcccaaaggc

SEQ ID NO: 14 - DNA, 382 bp, Artificial Sequence
  synthetic sequence
  misc_feature (198)..(221): each n is any nucleotide
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctgaagttaccgaagactctctgcgtctgtcttggnnnnnnnnnnnnnnnnnnnnnnnnttygactctttcctgatccagtaccaggaatctgaaaaagttggtgaagcgatcaacctgaccgttccgggttctgaacgttcttacgacctgaccggtctgaaaccgggtaccgaatacaccgtttctatctacggtgttcttagaagcttcccaaaggc

SEQ ID NO: 15 - DNA, 379 bp, Artificial Sequence
  synthetic sequence
  misc_feature (198)..(218): each n is any nucleotide
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctgaagttaccgaagactctctgcgtctgtcttggnnnnnnnnnnnnnnnnnnnnnttygactctttcctgatccagtaccaggaatctgaaaaagttggtgaagcgatcaacctgaccgttccgggttctgaacgttcttacgacctgaccggtctgaaaccgggtaccgaatacaccgtttctatctacggtgttcttagaagcttcccaaaggc

SEQ ID NO: 16 - DNA, 376 bp, Artificial Sequence
  synthetic sequence
  misc_feature (198)..(215): each n is any nucleotide
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctgaagttaccgaagactctctgcgtctgtcttggnnnnnnnnnnnnnnnnnnttygactctttcctgatccagtaccaggaatctgaaaaagttggtgaagcgatcaacctgaccgttccgggttctgaacgttcttacgacctgaccggtctgaaaccgggtaccgaatacaccgtttctatctacggtgttcttagaagcttcccaaaggc

SEQ ID NO: 17 - DNA, 131 bp, Artificial Sequence
  synthetic sequence
cggcggttagaacgcggctacaattaatacataaccccatccccctgttgacaattaatcatcggctcgtataatgtgtggaattgtgagcggataacaatttcacacaggaaacaggatctaccatgctg

SEQ ID NO: 18 - DNA, 30 bp, Artificial Sequence; identical to SEQ ID NO: 11
  synthetic sequence

SEQ ID NO: 19 - DNA, 81 bp, Artificial Sequence
  synthetic sequence
ccaagacagacgggcagagtcttcggtaacgcgagaaacaaccaggtttttcggcgccggcagcatggtagatcctgtttc

SEQ ID NO: 20 - DNA, 26 bp, Artificial Sequence
  synthetic sequence
ccgaagactctgcccgtctgtcttgg

SEQ ID NO: 21 - DNA, 45 bp, Artificial Sequence
  synthetic sequence
cagtggtctcacggattcctggtactggatcaggaaagagtcgaa

SEQ ID NO: 22 - DNA, 54 bp, Artificial Sequence
  synthetic sequence
catgcggtctcttccgaaaaagttggtgaagcgatcgtcctgaccgttccgggt

SEQ ID NO: 23 - DNA, 29 bp, Artificial Sequence
  synthetic sequence
ggtggtgaagatcgcagacagcgggttag

SEQ ID NO: 24 - DNA, 21 bp, Artificial Sequence
  synthetic sequence
cggcggttagaacgcggctac

SEQ ID NO: 25 - DNA, 61 bp, Artificial Sequence
  synthetic sequence
aagatcagttgcggccgctagactagaaccgctgccaccgccggtggtgaagatcgcagac

SEQ ID NO: 26 - DNA, 485 bp, Artificial Sequence
  synthetic sequence
  misc_feature (357)..(392): each n is any nucleotide
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 27 - DNA, 482 bp, Artificial Sequence
  synthetic sequence
  misc_feature (357)..(389): each n is any nucleotide
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 28 - DNA, 479 bp, Artificial Sequence
  synthetic sequence
  misc_feature (357)..(386): each n is any nucleotide
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 29 - DNA, 476 bp, Artificial Sequence
  synthetic sequence
  misc_feature (357)..(383): each n is any nucleotide
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 30 - DNA, 473 bp, Artificial Sequence
  synthetic sequence
  misc_feature (357)..(380): each n is any nucleotide
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 31 - DNA, 470 bp, Artificial Sequence
  synthetic sequence
  misc_feature (357)..(377): each n is any nucleotide
gtgacacggcggttagaacgcggctacaattaatacataaccccatccccctgttgacaattaatcatcggctcgtataatgtgtggaattgtgagcggataacaatttcacacaggaaacaggatctaccatgctgccggcgccgaaaaacctggttgtttctcgcgttaccgaagactctgcgcgtctgtcttggaccgcgccggacgcggcgttcgactctttcctgatccagtaccaggaatctgaaaaagttggtgaagcgatcgtgctgaccgttccgggttctgaacgttcttacgacctgaccggtctgaaaccgggtaccgaatacaccgtttctatctacggtgttnnnnnnnnnnnnnnnnnnnnntctaacccgctgtctgcgatcttcaccaccggcggtcaccatcaccatcaccatggcagcggttctagtctagcggccgcaactgatcttggc

SEQ ID NO: 32 - protein, 707 aa, Artificial Sequence
  synthetic sequence
KSSSEATNITPKHNMKAFLDELKAENIKKFLHNFTQIPHLAGTEQNFQLAKQIQSQWKEFGLDSVELTHYDVLLSYPNKTHPNYISIINEDGNEIFNTSLFEPPPAGYENVSDIVPPFSAFSPQGMPEGDLVYVNYARTEDFFKLERDMKINCSGKIVIARYGKVFRGNKVKNAQLAGATGVILYSDPDDYFAPGVKSYPDGWNLPGGGVQRGNILNLNGAGDPLTPGYPANEYAYRRGMAEAVGLPSIPVHPIGYYDAQKLLEKMGGSASPDSSWRGSLKVPYNVGPGFTGNFSTQKVKMHIHSTSEVTRIYNVIGTLRGAVEPDRYVILGGHRDSWVFGGIDPQSGAAVVHEIVRSFGMLKKEGWRPRRTILFASWDAEEFGLLGSTEWAEENSRLLQERGVAYINADSSIEGNYTLRVDCTPLMYSLVYNLTKELESPDEGFEGKSLYESWTKKSPSPEFSGMPRISKLGSGNDFEVFFQRLGIASGRARYTKNWETNKFSSYPLYHSVYETYELVEKFYDPMFKYHLTVAQVRGGMVFELANSVVLPFDCRDYAVVLRKYADKIYNISMKHPQEMKTYSVSFDSLFSAVKNFTEIASKFSERLRDFDKSNPILLRMMNDQLMFLERAFIDPLGLPDRPFYRHVIYAPSSHNKYAGESFPGIYDALFDIESKVDPSQAWGEVKRQISIATFTVQAAAETLSEVA

SEQ ID NO: 33 - protein, 707 aa, Artificial Sequence
  synthetic sequence
KSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVLDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRHGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVYNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYNISMKHPQEMKTYSVSFDSLFSAVKNFTEIASKFTERLQDFDKSNPILLRMMNDQLMFLERAFIDPLGLPDRPFYRHVIYAPSSHNKYAGESFPGIYDALFDIESKVDPSKAWGDVKRQISVAAFTVQAAAETLSEVA

SEQ ID NO: 34 - protein, 6 aa, Artificial Sequence
  synthetic sequence
HHHHHH

SEQ ID NO: 35 - protein, 91 aa, Artificial Sequence
  synthetic sequence
LPAPKNLVVSRVTEDSARLSWDIDEQRDWFDSFLIQYQESEKVGEAIVLTVPGSERSYDLTGLKPGTEYTVSIYGVYHVYRSNPLSAIFTT

SEQ ID NO: 36 - protein, 91 aa, Artificial Sequence
  synthetic sequence
LPAPKNLVVSRVTEDSARLSWTIDEQRDWFDSFLIQYQESEKVGEAIVLTVPGSERSYDLTGLKPGTEYTVSIYGVYHVYRSNPLSAIFTT

SEQ ID NO: 37 - protein, 91 aa, Artificial Sequence
  synthetic sequence
LPAPKNLVVSRVTEDSARLSWVIDEQRDWFDSFLIQYQESEKVGEAIVLTVPGSERSYDLTGLKPGTEYTVSIYGVYHVYRSNPLSAIFTT

SEQ ID NO: 38 - protein, 91 aa, Artificial Sequence
  synthetic sequence
LPAPKNLVVSRVTEDSARLSWTIDEQRDWFESFLIQYQESEKVGEAIVLTVPGSERSYDLTGLKPGTEYTVSIYGVYHVYRSNPLSAIFTT

SEQ ID NO: 39 - protein, 91 aa, Artificial Sequence
  synthetic sequence
LPAPKNLVVSRVTEDSARLSWAIDEQRDWFESFLIQYQESEKVGEAIVLTVPGSERSYDLTGLKPGTEYTVSIYGVYHVYRSNPLSAIFTT

SEQ ID NO: 40 - protein, 92 aa, Artificial Sequence
  synthetic sequence
LPAPKNLVVSRVTEDSARLSWDIDEQRDWFDSFLIQYQESEKVGEAIVLTVPGSERSYDLTGLKPGTEYTVSIYGVYHVYRSSNPLSAIFTT

SEQ ID NO: 41 - protein, 89 aa, Artificial Sequence
  synthetic sequence
LPAPKNLVVSRVTEDSARLSWTAPDAAFDSFAIGYWEWDDDGEAIVLTVPGSERSYDLTGLKPGTEYPVYIAGVKGGQWSFPLSAIFTT

SEQ ID NO: 42 - protein, 89 aa, Artificial Sequence
  synthetic sequence
LPAPKNLVVSRVTEDSARLSWTAPDAAFDSFTIGYWEWDDDGEAIVLTVPGSERSYDLTGLKPGTEYPVYIAGVKGGQWSFPLSAIFTT

SEQ ID NO: 43 - protein, 89 aa, Artificial Sequence
  synthetic sequence
LPAPKNLVVSRVTEDSARLSWTAPDAAFDSFPIGYWEWDDDGEAIVLTVPGSERSYDLTGLKPGTEYHVYIAGVKGGQWSFPLSAIFTT

SEQ ID NO: 44 - protein, 89 aa, Artificial Sequence
  synthetic sequence
LPAPKNLVVSRVTEDSARLSWTAPDAAFDSFPIGYWEWDDDGEAIVLTVPGSERSYDLTGLKPGTEYWVYIAGVKGGQWSFPLSAIFTT

SEQ ID NO: 45 - protein, 95 aa, Artificial Sequence
  synthetic sequence
LPAPKNLVVSRVTEDSARLSWGEQFTIFDSFLIQYQESEKVGEAIVLTVPGSERSYDLTGLKPGTEYTVSIYGASGYEWFHAFGSSNPLSAIFTT

SEQ ID NO: 46 - protein, 95 aa, Artificial Sequence
  synthetic sequence
LPAPKNLVVSRVTEDSARLSWEWWVIPGDFDSFLIQYQESEKVGEAIVLTVPGSERSYDLTGLKPGTEYTVSIYGVVNSGQWNDTSNPLSAIFTT

SEQ ID NO: 47 - protein, 90 aa, Artificial Sequence
  synthetic sequence
LPAPKNLVVSRVTEDSARLSWTAPDAAFDSFAIGYWEWDDDGEAIVLTVPGSERSYDLTGLKPGTEYPVYIAGVKGGQWSFPLSAIFTTC

SEQ ID NO: 48 - protein, 89 aa, Artificial Sequence
  synthetic sequence
LPAPKNLVVSRVTEDSARLSWTAPDAAFDSFAIGYWEWDDDGEAIVLTVPGSERSYDLTGLCPGTEYPVYIAGVKGGQWSFPLSAIFTT

SEQ ID NO: 49 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYPVYIAGVKGGQWSFPLSAIFTT

SEQ ID NO: 50 - protein, 89 aa, Artificial Sequence
  synthetic sequence
LPAPKNLVVSCVTEDSARLSWTAPDAAFDSFAIGYWEWDDDGEAIVLTVPGSERSYDLTGLKPGTEYPVYIAGVKGGQWSFPLSAIFTT

SEQ ID NO: 51 - protein, 97 aa, Artificial Sequence
  synthetic sequence
LPAPKNLVVSEVTEDSARLSWTAPDAAFDSFLIQYQESEKVGEAIVLTVPGSERSYDLTGLCPGTEYTVSIYGVKGGHRSNPLSAIFTTGGHHHHHH

SEQ ID NO: 52 - protein, 199 aa, Artificial Sequence
  synthetic sequence
MSHHHHHHSSGENLYFQSKPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVK

SEQ ID NO: 53 - protein, 182 aa, Artificial Sequence
  synthetic sequence
SKPHIDNYLHDKDKDEKIEQYDKNVKEQASKDKKQQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVK

SEQ ID NO: 54 - protein, 7 aa, Artificial Sequence
  synthetic sequence
ENLYFQS

SEQ ID NO: 55 - protein, 7 aa, Artificial Sequence
  synthetic sequence
KGGHRSN

SEQ ID NO: 56 - protein, 8 aa, Artificial Sequence
  synthetic sequence
DIDEQRDW

SEQ ID NO: 57 - protein, 8 aa, Artificial Sequence
  synthetic sequence
TIDEQRDW

SEQ ID NO: 58 - protein, 8 aa, Artificial Sequence
  synthetic sequence
VIDEQRDW

SEQ ID NO: 59 - protein, 8 aa, Artificial Sequence
  synthetic sequence
AIDEQRDW

SEQ ID NO: 60 - protein, 8 aa, Artificial Sequence
  synthetic sequence
EWWVIPGD

SEQ ID NO: 61 - protein, 6 aa, Artificial Sequence
  synthetic sequence
GEQFTI

SEQ ID NO: 62 - protein, 6 aa, Artificial Sequence
  synthetic sequence
TAPDAA

SEQ ID NO: 63 - protein, 10 aa, Artificial Sequence
  synthetic sequence
FDSFLIQYQE

SEQ ID NO: 64 - protein, 10 aa, Artificial Sequence
  synthetic sequence
FESFLIQYQE

SEQ ID NO: 65 - protein, 10 aa, Artificial Sequence
  synthetic sequence
FDSFAIGYWE

SEQ ID NO: 66 - protein, 10 aa, Artificial Sequence
  synthetic sequence
FDSFPIGYWE

SEQ ID NO: 67 - protein, 10 aa, Artificial Sequence
  synthetic sequence
FDSFTIGYWE

SEQ ID NO: 68 - protein, 6 aa, Artificial Sequence
  synthetic sequence
SEKVGE

SEQ ID NO: 69 - protein, 6 aa, Artificial Sequence
  synthetic sequence
WDDDGE

SEQ ID NO: 70 - protein, 10 aa, Artificial Sequence
  synthetic sequence
TEYTVSIYGV

SEQ ID NO: 71 - protein, 9 aa, Artificial Sequence
  synthetic sequence
TEYTVSIYG

SEQ ID NO: 72 - protein, 10 aa, Artificial Sequence
  synthetic sequence
TEYPVYIAGV

SEQ ID NO: 73 - protein, 10 aa, Artificial Sequence
  synthetic sequence
TEYWVYIAGV

SEQ ID NO: 74 - protein, 10 aa, Artificial Sequence
  synthetic sequence
TEYHVYIAGV

SEQ ID NO: 75 - protein, 8 aa, Artificial Sequence
  synthetic sequence
YHVYRSSN

SEQ ID NO: 76 - protein, 7 aa, Artificial Sequence
  synthetic sequence
YHVYRSN

SEQ ID NO: 77 - protein, 11 aa, Artificial Sequence
  synthetic sequence
VNSGQWNDTSN

SEQ ID NO: 78 - protein, 14 aa, Artificial Sequence
  synthetic sequence
ASGYEWFHAFGSSN

SEQ ID NO: 79 - protein, 7 aa, Artificial Sequence
  synthetic sequence
KGGQWSF

SEQ ID NO: 80 - protein, 91 aa, Artificial Sequence
  synthetic sequence
LPAPKNLVVSRVTEDSARLSWAIDEQRAWFESFLIQYQESEKVGEAIVLTVPGSCRSYDLTGLKPGTEYTVSIYGVYHVYRSNPLSAIFTT

SEQ ID NO: 81 - protein, 91 aa, Artificial Sequence
  synthetic sequence
LPAPKNLVVSRVTEDSARLSWAIDEQRDAFESFLIQYQESEKVGEAIVLTVPGSCRSYDLTGLKPGTEYTVSIYGVYHVYRSNPLSAIFTT

SEQ ID NO: 82 - protein, 91 aa, Artificial Sequence
  synthetic sequence
LPAPKNLVVSRVTEDSARLSWAIDEQRDWFESFLIQYQESEKVGEAIVLTVPGSCRSYDLTGLKPGTEYTVSIYGVAHVYRSNPLSAIFTT

SEQ ID NO: 83 - protein, 91 aa, Artificial Sequence
  synthetic sequence
LPAPKNLVVSRVTEDSARLSWAIDEQRDWFESFLIQYQESEKVGEAIVLTVPGSCRSYDLTGLKPGTEYTVSIYGVYAVYRSNPLSAIFTT

SEQ ID NO: 84 - protein, 91 aa, Artificial Sequence
  synthetic sequence
LPAPKNLVVSRVTEDSARLSWAIDEQRDWFESFLIQYQESEKVGEAIVLTVPGSCRSYDLTGLKPGTEYTVSIYGVYHAYRSNPLSAIFTT

SEQ ID NO: 85 - protein, 91 aa, Artificial Sequence
  synthetic sequence
LPAPKNLVVSRVTEDSARLSWAIDEQRDWFESFLIQYQESEKVGEAIVLTVPGSCRSYDLTGLKPGTEYTVSIYGVYHVARSNPLSAIFTT

SEQ ID NO: 86 - protein, 91 aa, Artificial Sequence
  synthetic sequence
LPAPKNLVVSRVTEDSARLSWAIDEQRDWFESFLIQYQESEKVGEAIVLTVPGSCRSYDLTGLKPGTEYTVSIYGVYHVYASNPLSAIFTT

SEQ ID NO: 87 - protein, 91 aa, Artificial Sequence
  synthetic sequence
LPAPKNLVVSRVTEDSARLSWAIDEQRDWFASFLIQYQESEKVGEAIVLTVPGSCRSYDLTGLKPGTEYTVSIYGVYHVYRSNPLSAIFTT

SEQ ID NO: 88 - protein, 91 aa, Artificial Sequence
  synthetic sequence
LPAPKNLVVSRVTEDSARLSWDIDEQRDWFESFLIQYQESEKVGEAIVLTVPGSCRSYDLTGLKPGTEYTVSIYGVYHVYRSNPLSAIFTT

SEQ ID NO: 89 - protein, 91 aa, Artificial Sequence
  synthetic sequence
LPAPKNLVVSRVTEDSARLSWAIDEQRDWFDSFLIQYQESEKVGEAIVLTVPGSCRSYDLTGLKPGTEYTVSIYGVYHVYRSNPLSAIFTT

SEQ ID NO: 90 - protein, 92 aa, Artificial Sequence
  synthetic sequence
LPAPKNLVVSRVTEDSARLSWAIDEQRDWFESFLIQYQESEKVGEAIVLTVPGSCRSYDLTGLKPGTEYTVSIYGVYHVYRSSNPLSAIFTT

SEQ ID NO: 91 - protein, 91 aa, Artificial Sequence
  synthetic sequence
LPAPKNLVVSRVTEDSARLSWDIDEQRDWFDSFLIQYQESEKVGEAIVLTVPGSCRSYDLTGLKPGTEYTVSIYGVYHVYRSNPLSAIFTT

SEQ ID NO: 92 - protein, 92 aa, Artificial Sequence
  synthetic sequence
LPAPKNLVVSRVTEDSARLSWDIDEQRDWFESFLIQYQESEKVGEAIVLTVPGSCRSYDLTGLKPGTEYTVSIYGVYHVYRSSNPLSAIFTT

SEQ ID NO: 93 - protein, 92 aa, Artificial Sequence
  synthetic sequence
LPAPKNLVVSRVTEDSARLSWAIDEQRDWFDSFLIQYQESEKVGEAIVLTVPGSCRSYDLTGLKPGTEYTVSIYGVYHVYRSSNPLSAIFTT

SEQ ID NO: 94 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYRVYIAGVKGGQWSFPLSAIFTT

SEQ ID NO: 95 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYKVYIAGVKGGQWSFPLSAIFTT

SEQ ID NO: 96 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYEVYIAGVKGGQWSFPLSAIFTT

SEQ ID NO: 97 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYHVYIAGVKGGQWSFPLSAIFTT

SEQ ID NO: 98 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYDVYIAGVKGGQWSFPLSAIFTT

SEQ ID NO: 99 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYAVYIAGVKGGQWSFPLSAIFTT

SEQ ID NO: 100 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYGVYIAGVKGGQWSFPLSAIFTT

SEQ ID NO: 101 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYVVYIAGVKGGQWSFPLSAIFTT

SEQ ID NO: 102 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYLVYIAGVKGGQWSFPLSAIFTT

SEQ ID NO: 103 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYIVYIAGVKGGQWSFPLSAIFTT

SEQ ID NO: 104 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYFVYIAGVKGGQWSFPLSAIFTT

SEQ ID NO: 105 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYWVYIAGVKGGQWSFPLSAIFTT

SEQ ID NO: 106 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYNVYIAGVKGGQWSFPLSAIFTT

SEQ ID NO: 107 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYQVYIAGVKGGQWSFPLSAIFTT

SEQ ID NO: 108 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYSVYIAGVKGGQWSFPLSAIFTT

SEQ ID NO: 109 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYTVYIAGVKGGQWSFPLSAIFTT

SEQ ID NO: 110 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYYVYIAGVKGGQWSFPLSAIFTT

SEQ ID NO: 111 - protein, 89 aa, Artificial Sequence
  synthetic sequence
LPAPKNLVVSRVTEDSARLSWTAPDAAFDSFAIAYWEWDDDGEAIVLTVPGSCRSYDLTGLKPGTEYPVYIAGVKGGQWSFPLSAIFTT

SEQ ID NO: 112 - protein, 89 aa, Artificial Sequence
  synthetic sequence
LPAPKNLVVSRVTEDSARLSWTAPDAAFDSFAISYWEWDDDGEAIVLTVPGSCRSYDLTGLKPGTEYPVYIAGVKGGQWSFPLSAIFTT

SEQ ID NO: 113 - protein, 89 aa, Artificial Sequence
  synthetic sequence
LPAPKNLVVSRVTEDSARLSWTAPDAAFDSFAIGYWEWDTDGEAIVLTVPGSCRSYDLTGLKPGTEYPVYIAGVKGGQWSFPLSAIFTT

SEQ ID NO: 114 - protein, 89 aa, Artificial Sequence
  synthetic sequence
LPAPKNLVVSRVTEDSARLSWTAPDAAFDSFAIGYWEWDSDGEAIVLTVPGSCRSYDLTGLKPGTEYPVYIAGVKGGQWSFPLSAIFTT

SEQ ID NO: 115 - protein, 89 aa, Artificial Sequence
  synthetic sequence
LPAPKNLVVSRVTEDSARLSWTAPDAAFDSFAIGYYEWDDDGEAIVLTVPGSCRSYDLTGLKPGTEYPVYIAGVKGGQWSFPLSAIFTT

SEQ ID NO: 116 - protein, 89 aa, Artificial Sequence
  synthetic sequence
LPAPKNLVVSRVTEDSARLSWTAPDAAFDSFAIGYFEWDDDGEAIVLTVPGSCRSYDLTGLKPGTEYPVYIAGVKGGQWSFPLSAIFTT

SEQ ID NO: 117 - protein, 89 aa, Artificial Sequence
  synthetic sequence
LPAPKNLVVSRVTEDSARLSWTAPDAAFDSFAIGYLEWDDDGEAIVLTVPGSCRSYDLTGLKPGTEYPVYIAGVKGGQWSFPLSAIFTT

SEQ ID NO: 118 - protein, 89 aa, Artificial Sequence
  synthetic sequence
LPAPKNLVVSRVTEDSARLSWTAPDAAFDSFAIGYWEYDDDGEAIVLTVPGSCRSYDLTGLKPGTEYPVYIAGVKGGQWSFPLSAIFTT

SEQ ID NO: 119 - protein, 89 aa, Artificial Sequence
  synthetic sequence
LPAPKNLVVSRVTEDSARLSWTAPDAAFDSFAIGYWEFDDDGEAIVLTVPGSCRSYDLTGLKPGTEYPVYIAGVKGGQWSFPLSAIFTT

SEQ ID NO: 120 - protein, 89 aa, Artificial Sequence
  synthetic sequence
LPAPKNLVVSRVTEDSARLSWTAPDAAFDSFAIGYWELDDDGEAIVLTVPGSCRSYDLTGLKPGTEYPVYIAGVKGGQWSFPLSAIFTT

SEQ ID NO: 121 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYPVYIAGVKGGQYSFPLSAIFTT

SEQ ID NO: 122 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYPVYIAGVKGGQFSFPLSAIFTT

SEQ ID NO: 123 - protein, 89 aa, Artificial Sequence
  synthetic sequence
LPAPKNLVVSRVTEDSARLSWTAPDAAFDSFAIGYWEWDDDGEAIVLTVPGSCRSYDLTGLKPGTEYPVYIAGVKGGQLSFPLSAIFTT

SEQ ID NO: 124 - protein, 89 aa, Artificial Sequence
  synthetic sequence
LPAPKNLVVSRVTEDSARLSWTAPDAAFDSFRIGYWEWDDDGEAIVLTVPGSCRSYDLTGLKPGTEYHVYIAGVKGGQWSFPLSAIFTT

SEQ ID NO: 125 - protein, 89 aa, Artificial Sequence
  synthetic sequence
LPAPKNLVVSRVTEDSARLSWTAPDAAFDSFKIGYWEWDDDGEAIVLTVPGSCRSYDLTGLKPGTEYHVYIAGVKGGQWSFPLSAIFTT

SEQ ID NO: 126 - protein, 89 aa, Artificial Sequence
  synthetic sequence
LPAPKNLVVSRVTEDSARLSWTAPDAAFDSFEIGYWEWDDDGEAIVLTVPGSCRSYDLTGLKPGTEYHVYIAGVKGGQWSFPLSAIFTT

SEQ ID NO: 127 - protein, 89 aa, Artificial Sequence
  synthetic sequence
LPAPKNLVVSRVTEDSARLSWTAPDAAFDSFHIGYWEWDDDGEAIVLTVPGSCRSYDLTGLKPGTEYHVYIAGVKGGQWSFPLSAIFTT

SEQ ID NO: 128 - protein, 89 aa, Artificial Sequence
  synthetic sequence
LPAPKNLVVSRVTEDSARLSWTAPDAAFDSFDIGYWEWDDDGEAIVLTVPGSCRSYDLTGLKPGTEYHVYIAGVKGGQWSFPLSAIFTT

SEQ ID NO: 129 - protein, 89 aa, Artificial Sequence; identical to SEQ ID NO: 97
  synthetic sequence

SEQ ID NO: 130 - protein, 89 aa, Artificial Sequence
  synthetic sequence
LPAPKNLVVSRVTEDSARLSWTAPDAAFDSFGIGYWEWDDDGEAIVLTVPGSCRSYDLTGLKPGTEYHVYIAGVKGGQWSFPLSAIFTT

SEQ ID NO: 131 - protein, 89 aa, Artificial Sequence
  synthetic sequence
LPAPKNLVVSRVTEDSARLSWTAPDAAFDSFVIGYWEWDDDGEAIVLTVPGSCRSYDLTGLKPGTEYHVYIAGVKGGQWSFPLSAIFTT

SEQ ID NO: 132 - protein, 89 aa, Artificial Sequence
  synthetic sequence
LPAPKNLVVSRVTEDSARLSWTAPDAAFDSFLIGYWEWDDDGEAIVLTVPGSCRSYDLTGLKPGTEYHVYIAGVKGGQWSFPLSAIFTT

SEQ ID NO: 133 - protein, 89 aa, Artificial Sequence
  synthetic sequence
LPAPKNLVVSRVTEDSARLSWTAPDAAFDSFIIGYWEWDDDGEAIVLTVPGSCRSYDLTGLKPGTEYHVYIAGVKGGQWSFPLSAIFTT

SEQ ID NO: 134 - protein, 89 aa, Artificial Sequence
  synthetic sequence
LPAPKNLVVSRVTEDSARLSWTAPDAAFDSFFIGYWEWDDDGEAIVLTVPGSCRSYDLTGLKPGTEYHVYIAGVKGGQWSFPLSAIFTT

SEQ ID NO: 135 - protein, 89 aa, Artificial Sequence
  synthetic sequence
LPAPKNLVVSRVTEDSARLSWTAPDAAFDSFWIGYWEWDDDGEAIVLTVPGSCRSYDLTGLKPGTEYHVYIAGVKGGQWSFPLSAIFTT

SEQ ID NO: 136 - protein, 89 aa, Artificial Sequence
  synthetic sequence
LPAPKNLVVSRVTEDSARLSWTAPDAAFDSFNIGYWEWDDDGEAIVLTVPGSCRSYDLTGLKPGTEYHVYIAGVKGGQWSFPLSAIFTT

SEQ ID NO: 137 - protein, 89 aa, Artificial Sequence
  synthetic sequence
LPAPKNLVVSRVTEDSARLSWTAPDAAFDSFQIGYWEWDDDGEAIVLTVPGSCRSYDLTGLKPGTEYHVYIAGVKGGQWSFPLSAIFTT

SEQ ID NO: 138 - protein, 89 aa, Artificial Sequence
  synthetic sequence
LPAPKNLVVSRVTEDSARLSWTAPDAAFDSFSIGYWEWDDDGEAIVLTVPGSCRSYDLTGLKPGTEYHVYIAGVKGGQWSFPLSAIFTT

SEQ ID NO: 139 - protein, 89 aa, Artificial Sequence
  synthetic sequence
LPAPKNLVVSRVTEDSARLSWTAPDAAFDSFTIGYWEWDDDGEAIVLTVPGSCRSYDLTGLKPGTEYHVYIAGVKGGQWSFPLSAIFTT

SEQ ID NO: 140 - protein, 89 aa, Artificial Sequence
  synthetic sequence
LPAPKNLVVSRVTEDSARLSWTAPDAAFDSFYIGYWEWDDDGEAIVLTVPGSCRSYDLTGLKPGTEYHVYIAGVKGGQWSFPLSAIFTT

SEQ ID NO: 141 - protein, 700 aa, Artificial Sequence
  synthetic sequence
MWNLLHETDSAVATARRPRWLCAGALVLAGGFFLLGFLFGWFIKSSSEATNITPKHNMKAFLDELKAENIKKFLHNFTQIPHLAGTEQNFQLAKQIQSQWKEFGLDSVELTHYDVLLSYPNKTHPNYISIINEDGNEIFNTSLFEPPPAGYENVSDIVPPFSAFSPQGMPEGDLVYVNYARTEDFFKLERDMKINCSGKIVIARYGKVFRGNKVKNAQLAGATGVILYSDPDDYFAPGVKSYPDGWNLPGGGVQRGNILNLNGAGDPLTPGYPANEYAYRRGMAEAVGLPSIPVHPIGYYDAQKLLEKMGGSASPDSSWRGSLKVPYNVGPGFTGNFSTQKVKMHIHSTSEVTRIYNVIGTLRGAVEPDRYVILGGHRDSWVFGGIDPQSGAAVVHEIVRSFGMLKKEGWRPRRTILFASWDAEEFGLLGSTEWAEENSRLLQERGVAYINADSSIEGNYTLRVDCTPLMYSLVYNLTKELESPDEGFEGKSLYESWTKKSPSPEFSGMPRISKLGSGNDFEVFFQRLGIASGRARYTKNWETNKFSSYPLYHSVYETYELVEKFYDPMFKYHLTVAQVRGGMVFELANSVVLPFDCRDYAVVLRKYADKIYNISMKHPQEMKTYSVSFDSLFSAVKNFTEIASKFSERLRDFDKSNPILLRMMNDQLMFLERAFIDPLGLPDRPFYRHVIYAPSSHNKY

SEQ ID NO: 142 - protein, 29 aa, Artificial Sequence
  synthetic sequence
AEAAAKEAAAKEAAAKEAAAKEAAAKAAA

SEQ ID NO: 143 - protein, 707 aa, Homo sapiens
KSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 144 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 145 - protein, 88 aa, Artificial Sequence
  synthetic sequence
DAPSQIEVKDVTDTTALITWFKPLAEIDGIELTYGIKDVPGDRTTIDLTEDENQYSIGNLKPDTEYEVSLISRRGDMSSNPAKETFTT

SEQ ID NO: 146 - protein, 89 aa, Artificial Sequence
  synthetic sequence
LDAPTDLQVTNVTDTSITVSWTPPSATITGYRITYTPSNGPGEPKELTVPPSSTSVTITGLTPGVEYVVSLYALKDNQESPPLVGTQTT

SEQ ID NO: 147 - protein, 94 aa, Artificial Sequence
  synthetic sequence
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 148 - protein, 4 aa, Artificial Sequence
  synthetic sequence
GSGS

SEQ ID NO: 149 - protein, 8 aa, Artificial Sequence
  synthetic sequence
GGGSGGGS

SEQ ID NO: 150 - protein, 25 aa, Artificial Sequence
  synthetic sequence
GGGGSGGGGSGGGGSGGGGSGGGGS

SEQ ID NO: 151 - protein, 4 aa, Artificial Sequence
  synthetic sequence
APAP

SEQ ID NO: 152 - protein, 10 aa, Artificial Sequence
  synthetic sequence
APAPAPAPAP

SEQ ID NO: 153 - protein, 20 aa, Artificial Sequence
  synthetic sequence
APAPAPAPAPAPAPAPAPAP

SEQ ID NO: 154 - protein, 40 aa, Artificial Sequence
  synthetic sequence
APAPAPAPAPAPAPAPAPAPAPAPAPAPAPAPAPAPAPAP

SEQ ID NO: 155 - protein, 585 aa, Artificial Sequence
  synthetic sequence
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 156 - protein, 267 aa, Artificial Sequence
  synthetic sequence
CTGCCAGCCCCGAAGAATTTGGTCGTTTCCCGTGTCACTGAGGACTCTGCACGTCTGAGCTGGACCGCACCGGACGCGGCGTTCGACAGCTTTGCAATCGGCTACTGGGAGTGGGATGATGACGGCGAGGCCATTGTGCTGACCGTTCCGGGTAGCGAGCGCAGCTACGATCTGACCGGTCTGAAGCCGGGTACGGAATATCCGGTGTATATTGCGGGCGTGAAGGGTGGCCAGTGGAGCTTCCCGCTGAGCGCGATCTTTACCACC

SEQ ID NO: 157 - protein, 273 aa, Artificial Sequence
  synthetic sequence
CTGCCGGCTCCGAAAAACCTGGTCGTTTCCCGTGTCACTGAAGATTCTGCACGCTTGAGCTGGGCGATCGACGAGCAGCGTGACTGGTTTGAGAGCTTCCTGATTCAGTATCAAGAATCGGAAAAAGTTGGCGAGGCCATCGTGCTGACCGTTCCGGGTAGCGAGCGCAGCTATGATCTGACGGGTCTGAAGCCAGGCACCGAGTATACGGTGAGCATTTACGGTGTCTACCATGTGTACCGTAGCAATCCGCTGAGCGCGATCTTCACCACC

SEQ ID NO: 158 - protein, 267 aa, Artificial Sequence
  synthetic sequence
CTGCCAGCCCCGAAAAACTTAGTTGTCTCCCGCGTGACCGAAGATTCTGCTCGTCTGAGCTGGACTGCACCGGACGCGGCGTTCGACAGCTTTCCGATTGGCTACTGGGAGTGGGATGATGACGGTGAAGCGATCGTGCTGACCGTTCCGGGTAGCGAGCGTAGCTATGACCTGACGGGTTTGAAACCTGGTACCGAGTATCACGTTTACATTGCGGGCGTCAAGGGTGGCCAGTGGTCGTTCCCGCTGAGCGCAATCTTTACGACC

SEQ ID NO: 159 - protein, 16 aa, Artificial Sequence
  synthetic sequence
KKSPSPEFSGMPRISK

SEQ ID NO: 160 - protein, 7 aa, Artificial Sequence
  synthetic sequence
NWETNKF

SEQ ID NO: 161 - protein, 201 aa, Artificial Sequence
  synthetic sequence
MLPAPKNLVVSRVTEDSARLSWAIDEQRDWFESFLIQYQESEKVGEAIVLTVPGSERSYDLTGLKPGTEYTVSIYGVYHVYRSNPLSAIFTTGGGGSGGGGSGGGGSGGGGSLPAPKNLVVSRVTEDSARLSWTAPDAAFDSFAIGYWEWDDDGEAIVLTVPGSERSYDLTGLKPGTEYPVYIAGVKGGQWSFPLSAIFTT

SEQ ID NO: 162 - protein, 16 aa, Artificial Sequence
  synthetic sequence
GGGSGGGSGGGSGGGS

SEQ ID NO: 163 - protein, 12 aa, Artificial Sequence
  synthetic sequence
GGGSGGGSGGGS

SEQ ID NO: 164 - protein, 21 aa, Artificial Sequence
  synthetic sequence
MALPVTALLLPLALLLHAARP

SEQ ID NO: 165 - protein, 69 aa, Artificial Sequence
  synthetic sequence
TTTPAPRPPTPAPTIASQPLSLRPEACRPAAGGAVHTRGLDFACDIYIWAPLAGTCGVLLLSLVITLYC

SEQ ID NO: 166 - protein, 179 aa, Artificial Sequence
  synthetic sequence
MALPVTALLLPLALLLHAARPLPAPKNLVVSRVTEDSARLSWTAPDAAFDSFAIGYWEWDDDGEAIVLTVPGSERSYDLTGLKPGTEYPVYIAGVKGGQWSFPLSAIFTTTTTPAPRPPTPAPTIASQPLSLRPEACRPAAGGAVHTRGLDFACDIYIWAPLAGTCGVLLLSLVITLYC

SEQ ID NO: 167 - protein, 179 aa, Artificial Sequence
  synthetic sequence
MALPVTALLLPLALLLHAARPLPAPKNLVVSRVTEDSARLSWAIDEQRDWFESFLIQYQESEKVGEAIVTVPGSERSYDLTGLKPGTEYTVSIYGVYHVYRSNPLSAIFTTTTTPAPRPPTPAPTIASQPLSLRPEACRPAAGGAVHTRGLDFACDIYIWAPLAGTCGVLLLSLVITLY

SEQ ID NO: 168 - protein, 89 aa, Artificial Sequence; identical to SEQ ID NO: 41
  synthetic sequence

SEQ ID NO: 169 - protein, 89 aa, Artificial Sequence; identical to SEQ ID NO: 41
  synthetic sequence

SEQ ID NO: 170 - protein, 194 aa, Artificial Sequence
  synthetic sequence
MALPVTALLLPLALLLHAARPLPAPKNLVVSRVTEDSARLSWTAPDAAFDSFAIGYWEWDDDGEAIVLTVPGSERSYDLTGLKPGTEYPVYIAGVKGGQWSFPLSAIFTTGGGGSGGGGSGGGGSTTTPAPRPPTPAPTIASQPLSLRPEACRPAAGGAVHTRGLDFACDIYIWAPLAGTCGVLLLSLVITLYC

SEQ ID NO: 171 - protein, 189 aa, Artificial Sequence
  synthetic sequence
MALPVTALLLPLALLLHAARPLPAPKNLVVSRVTEDSARLSWTAPDAAFDSFAIGYWEWDDDGEAIVLTVPGSERSYDLTGLKPGTEYPVYIAGVKGGQWSFPLSAIFTTGGGGSGGGGSTTTPAPRPPTPAPTIASQPLSLRPEACRPAAGGAVHTRGLDFACDIYIWAPLAGTCGVLLLSLVITLYC

SEQ ID NO: 172 - protein, 179 aa, Artificial Sequence
  synthetic sequence
MALPVTALLLPLALLLHAARPLPAPKNLVVSRVTEDSARLSWDIDEQRDWFESFLIQYQESEKVGEAIVTVPGSERSYDLTGLKPGTEYTVSIYGVYHVYRSNPLSAIFTTTTTPAPRPPTPAPTIASQPLSLRPEACRPAAGGAVHTRGLDFACDIYIWAPLAGTCGVLLLSLVITLY

SEQ ID NO: 173 - protein, 91 aa, Artificial Sequence
  synthetic sequence
LPAPKNLVVSRVTEDSARLSWAADEQRDWFESFLIQYQESEKVGEAIVLTVPGSCRSYDLTGLKPGTEYTVSIYGVYHVYRSNPLSAIFTT

SEQ ID NO: 174 - protein, 91 aa, Artificial Sequence
  synthetic sequence
LPAPKNLVVSRVTEDSARLSWAIAEQRDWFESFLIQYQESEKVGEAIVLTVPGSCRSYDLTGLKPGTEYTVSIYGVYHVYRSNPLSAIFTT

SEQ ID NO: 175 - protein, 91 aa, Artificial Sequence
  synthetic sequence
LPAPKNLVVSRVTEDSARLSWAIDAQRDWFESFLIQYQESEKVGEAIVLTVPGSCRSYDLTGLKPGTEYTVSIYGVYHVYRSNPLSAIFTT

SEQ ID NO: 176 - protein, 91 aa, Artificial Sequence
  synthetic sequence
LPAPKNLVVSRVTEDSARLSWAIDEARDWFESFLIQYQESEKVGEAIVLTVPGSCRSYDLTGLKPGTEYTVSIYGVYHVYRSNPLSAIFTT

SEQ ID NO: 177 - protein, 91 aa, Artificial Sequence
  synthetic sequence
LPAPKNLVVSRVTEDSARLSWAIDEQADWFESFLIQYQESEKVGEAIVLTVPGSCRSYDLTGLKPGTEYTVSIYGVYHVYRSNPLSAIFTT